(19) United States
(12) Patent Application Publication
(10) Pub. No.: US 2023/0227565 A1
(43) Pub. Date: Jul. 20, 2023

(54) Method for Treating CD127-Positive Cancers by Administering an Anti-CD127 Agent

(71) Applicant: OSE IMMUNOTHERAPEUTICS, NANTES (FR)

(72) Inventors: Nicolas POIRIER, Grandchamps des Fontaines (FR); Irène BACCELLI, Nantes (FR); Caroline MARY, Sainte-Pazanne (FR); Sabrina PENGAM, St Luce S/Loire (FR)

(21) Appl. No.: 18/060,129

(22) Filed: Nov. 30, 2022

Related U.S. Application Data
(63) Continuation-in-part of application No. PCT/IB2022/000365, filed on May 27, 2022.
Application No. PCT/IB2022/000365 is a continuation-in-part of application No. 17/334,158, filed on May 28, 2021.
Application No. 17/334,158 is a continuation-in-part of application No. 17/334,158, filed on May 28, 2021.

(51) Int. Cl. C07K 16/28; A61K 31/573; A61K 39/395; A61K 31/475; A61K 38/50; A61P 35/02

(52) U.S. Cl. CPC C07K 16/2866; A61K 31/573; A61K 39/3955; A61K 31/475; A61K 38/50; A61P 35/02; C07K 2317/24; C07K 2317/21

(57) ABSTRACT

New clinical uses of anti-CD127 agents, in particular anti-CD127 antibodies or related compounds for the treatment and/or the prevention of cancer. Methods for treating a patient having a CD127-positive cancer are provided, in particular a CD127-positive leukemia or a CD127-positive solid tumor, by administering to the patient a therapeutic dose of an anti-CD127 agent, the anti-CD127 agent having the capability to enhance the Antibody Dependent Cellular Phagocytosis (ADCP) activity of macrophages targeting CD127-positive cancer cells, and not having Antibody Dependent Cytotoxic Activity (ADCC), in particular against immune cells, more particularly against T cells.

### CROSS-REFERENCE TO RELATED APPLICATIONS

This patent application is a by-pass continuation-in-part (CIP) application of PCT/IB2022/000365, filed May 27, 2022, which claims the benefit of U.S. ordinary non-provisional application Ser. No. 17/334,158, filed May 28, 2021, both of which are incorporated herein in their entireties. This patent application is also a continuation-in-part (CIP) application of U.S. ordinary non-provisional application Ser. No. 17/334,158, filed May 28, 2021, which is incorporated herein in its entirety.

### INCORPORATION OF SEQUENCE LISTING

A sequence listing containing the file named “B14422WOUS-CIP—Sequence listing ST26.xml”, which is 44,913 bytes (measured in MS-Windows®), contains 32 sequences, and was created on November 28, 2022, is provided herewith and is incorporated herein by reference in its entirety.

### FIELD OF THE INVENTION

The field of the invention pertains to immunotherapy, including new clinical uses of anti-CD127 agents, and in particular anti-CD127 antibodies or related compounds for the treatment and/or the prevention of cancer.

### BACKGROUND OF THE INVENTION

Cancer is a major worldwide health concern causing the death of approximately 9,5 million people a year, while more than 20 million people develop a cancer within a year (world cancer report by World Health Organization, 2018). Several malignant cells have been shown to display CD127 expression. This is for example the case for Sezary cutaneous lymphoma (60% of them), or childhood acute lymphoblastic leukemia in which about 15% of the children develop gain-of-function mutation in CD127, rendering these tumors partially IL-7 dependent (Shochat et al., 2011). Acute lymphoblastic leukemia (ALL) is a cancer of the lymphoid line of blood cells characterized by the development of large numbers of immature lymphocytes. ALL progresses rapidly and is typically fatal within weeks or months if left untreated. The underlying mechanism involves multiple genetic mutations that results in rapid cell division. The excessive immature lymphocytes in the bone marrow interfere with the production of new red blood cells, white blood cells, and platelets.

ALL is typically treated initially with chemotherapy (Dexamethasone, Vincristine and PEG-Asparaginase, Malard and Mohty, The Lancet 2020). This is then followed by further chemotherapy typically over a number of years. Chemotherapy treatments usually cause several side effects including but not limited to fatigue, hair loss, easy bruising and bleeding, infection, anemia (low red blood cell counts), nausea and vomiting. Additional and/or different treatments of ALL may include intrathecal chemotherapy or radiation therapy. Stem cell transplantation may be used if the disease recurs following standard treatment.

Despite numerous existing treatments, there is still a need for an improved method of treating cancer. New treatments based on antibody technology such as chimeric antigen receptor T cell (CAR-T cell) immunotherapy and use of monoclonal antibodies are being used and further studied. Indeed, several strategies involving the administration of CAR-T cells, anti-CD3 and anti-CD19 bispecific antibodies are currently in development for treating patients developing an ALL. Nonetheless, these strategies have drawbacks, among which one can cite the cost of these therapies and mostly the toxicity associated with the administered compounds. Due to the ubiquitous expression of the target CD19 marker in all B-lineage subtypes, these strategies may lead to collateral damages within the host during the treatment of ALL. Accordingly, there is a need for new treatments of ALL, which do not have the same drawbacks as the treatment of the prior art.

CD127 is part of the heterodimeric IL-7 receptor that is composed of CD127 and the common γ chain, which is shared by other cytokine receptors (IL-2R, IL-4R, IL-9R, IL-15R, and IL-21R). CD127 is expressed on thymocytes, T- and B-cell progenitors, mature T cells, monocytes, and some other lymphoid and myeloid cells. Studies have shown that IL-7R plays an important role in the proliferation and differentiation of mature T cells. Further, signaling induced by the dimerization of CD127 with the common y chain plays a pivotal role in T-cell development and maintenance of T-cell memory. Expression of CD127 is commonly associated with central and effector memory functions in both CD4 and CD8 peripheral T cells.

Prior uses of anti-CD127 agents, including anti-CD127 antibodies, for treating diseases rely on the inhibition of the IL-7 signaling pathway by the agent. By preventing or inhibiting the IL7-IL-7R interaction, molecular signaling pathway induced by the interaction of IL-7 and IL-7R is not (fully) functional, thereby allowing the treatment of diseases wherein this pathway is directly or indirectly involved (see for example WO2018/104483 or WO2011/094259).

### SUMMARY

The invention provides for the first time the use of anti-CD127 agents to enhance the phagocytosis of CD127+ tumor cells through Antibody Dependent Cellular Phagocytosis mechanism. As illustrated in the examples of the invention, patients having a CD127+ cancer, including different types of cancer like leukemias such as Acute Lymphoblastic Leukemia but also solid cancers such as mesothelioma, are likely to positively respond to a treatment with an anti-CD127 agent as disclosed in the present description. While several anti-CD127 antibodies were known in the prior art for treating auto-immune diseases through the capability of the antibodies to inhibit the IL-7R signaling pathway in T cells, it is herein provided new clinical uses due to the capability of the anti-CD127 agent to enhance phagocytosis of tumor cells. The present invention does not rely on the effect of anti-CD127 agent on T cells as described in the prior art, but on other cells, allowing to target other diseases and cell populations (in particular tumors wherein the IL-7R signaling pathway is dysfunctional; a situation often met in the clinic).

The inventors have identified that anti-CD127 agents that have Antibody Dependent Cellular Phagocytosis (ADCP) activity against CD127-positive tumor cells, in particular by macrophages, and that do not have Antibody Dependent Cytotoxic Activity (ADCC), in particular against immune cells, more particularly against T cells, may be useful in the treatment of CD127-positive cancers, in particular in the treatment of CD127-positive ALL and in particular in the treatment of CD127-positive mesothelioma. By administering an anti-CD127 agent as defined, the inventors observed that phagocytosis of cancer cells by immune cells of the host is increased. Thus, the administration of an anti-CD127 agent that has Antibody Dependent Cellular Phagocytosis (ADCP) activity against CD127-positive tumor cells, in particular by macrophages, and that does not have Antibody Dependent Cytotoxic Activity (ADCC), in particular against immune cells, more particularly against T cells, to a patient in need thereof leads to the phagocytosis of cancer cells expressing CD127.

The invention relates to an anti-CD127 agent for use in the treatment of a patient having a CD127-positive cancer and a method for treating a patient having a CD127-positive cancer, in particular a CD127-positive leukemia or CD127-positive solid tumors, by administering to the patient a therapeutic dose of an anti-CD127 agent, the anti-CD127 agent having Antibody Dependent Cellular Phagocytosis (ADCP) activity against CD127-positive tumor cells, in particular by macrophages, and that does not have Antibody Dependent Cytotoxic Activity (ADCC), in particular against immune cells, more particularly against T cells.

The invention also relates to an anti-CD127 agent, in particular an anti-CD127 antibody or antigen-binding fragment thereof or antigen-binding antibody mimetic, for use as a medicament in the treatment of a patient having a CD127-positive cancer, in particular by enhancing the phagocytosis of CD127-positive tumor cells, more particularly by macrophages, wherein the anti-CD127 agent has Antibody Dependent Cellular Phagocytosis (ADCP) activity against CD127-positive tumor cells, in particular by macrophages, and does not have Antibody Dependent Cytotoxic Activity (ADCC), in particular against immune cells, more particularly against T cells.

The present invention also relates to a method for promoting phagocytosis of CD127-positive cells, in particular CD127-positive tumor cells, particularly by macrophages, by administering to a patient in need thereof a therapeutic amount of an anti-CD127 agent that has Antibody Dependent Cellular Phagocytosis (ADCP) activity against CD127-positive tumor cells, in particular by macrophages, and that does not have Antibody Dependent Cytotoxic Activity (ADCC), in particular against immune cells, more particularly against T cells.

The invention also provides an anti-CD127 agent that has Antibody Dependent Cellular Phagocytosis (ADCP) activity against CD127-positive cells, in particular tumor cells, in particular by macrophages, and that does not have Antibody Dependent Cytotoxic Activity (ADCC), in particular against immune cells, more particularly against T cells, for the treatment of a patient having CD127-positive tumor cells, in particular CD127-positive leukemia or CD127-positive solid tumor cells, more particularly having CD127-positive Acute Lymphoblastic Leukemia.

The invention also relates to an anti-CD127 agent for use in the treatment of a patient having a CD127-positive cancer and a method for treating a patient having a CD127-positive cancer, in particular a CD127-positive leukemia, by administering to the patient a therapeutic dose of an anti-CD127 agent, the anti-CD127 agent having Antibody Dependent Cellular Phagocytosis (ADCP) activity against CD127-positive tumor cells, in particular by macrophages, and preferably the anti-CD127 agent not having Antibody Dependent Cytotoxic Activity (ADCC), in particular against immune cells, more particularly against T cells.

The invention also provides an anti-CD127 agent which enhances Antibody Dependent Cellular Phagocytosis (ADCP) activity of macrophages targeting CD127-positive cancer cells, for the treatment of a patient having CD127-positive tumor cells, in particular CD127-positive leukemia or CD127-positive solid tumor cells, more particularly having CD127-positive Acute Lymphoblastic Leukemia, and preferably wherein the anti-CD127 agent does not have Antibody Dependent Cytotoxic Activity (ADCC), in particular against immune cells, more particularly against T cells.

The invention also relates to an anti-CD127 agent for use as a medicament for promoting phagocytosis of CD127-positive cells of a patient, in particular CD127-positive tumor cells, particularly by macrophages, the anti-CD127 agent having Antibody Dependent Cellular Phagocytosis (ADCP) activity against CD127-positive tumor cells, in particular by macrophages, and the anti-CD127 agent not having Antibody Dependent Cytotoxic Activity (ADCC), in particular against immune cells, more particularly against T cells.

The invention also relates to the use of an anti-CD127 agent for preparing a medicament for promoting phagocytosis of CD127-positive cells of a patient, in particular CD127-positive tumor cells, particularly by macrophages, the anti-CD127 agent having Antibody Dependent Cellular Phagocytosis (ADCP) activity against CD127-positive tumor cells, in particular by macrophages, and the anti-CD127 agent not having Antibody Dependent Cytotoxic Activity (ADCC), in particular against immune cells, more particularly against T cells.

### BRIEF DESCRIPTION OF THE DRAWINGS

FIGS. 1A,B,C. Minimal residual disease (MRD) eradication in patients derived xenografts (PDX) experiments. (A) and (B) correspond to the probability of survival of mice over days post-transplant of two PDX issued from two different pediatric patients having t(1;19) B-ALL. Mice are treated with two different anti-CD127 agents with ADCP capabilities (an antagonist anti-CD127 agent in red (N13B2-hVL6) and a neutral (i.e., not antagonist nor agonist) anti-CD127 agent in green, Effi-3-VH3VL3), and a negative control (blue). (C) indicates, in the context of 4 different T-ALL PDX, the absence of leukemic blasts in the peripheral blood of mice treated with N13B2-hVL6, as opposed to control mice, which all develop leukemias.

FIGS. 2A,B,C,D,E. Overt leukemia delayed development in patient derived xenografts (PDX) experiments. (A) and (B) correspond to the probability of survival of mice over days post-transplant of two overt leukemia PDX issued from two different pediatric patients having t(1;19) B-ALL. Results illustrated in (A1) and (B1) correspond to mice treated with an antagonist anti-CD127 agent with ADCP capabilities (red, N13B2-hVL6) and a negative control (blue). Results illustrated in (A2) and (B2) correspond to mice treated with a neutral (i.e., not antagonist nor agonist) anti-CD127 agent with ADCP capabilities (green, Effi-3-VH3VL3) and a negative control (blue). (C) and (D) correspond to the percentage of survival of mice treated with the ADCP+/ADCC− anti-CD127 agent N13B2-hVL6 (orange) and a negative control (blue) in overt leukemia PDX cohorts of B-ALL and T-ALL, respectively. (E) illustrates the correlation between in vivo efficacy (fold-change increase in survival time of N13B2-hVL6 treated mice compared to control treated mice) of an ADCP+/ADCC− anti-CD127 agent and CD127 expression levels detected in ALL patient derived xenografted cells.

FIG. 3. Quantification of specific antibody binding of N13B2-hVL6 in a panel of tumor cell lines issued from different kinds of Acute Lymphoblastic Leukemias. Jurkat, HPB-ALL and DND41 correspond to three different T-cell ALL cell lines. 697, NALM6 and REH correspond to three different B-ALL cell lines. The specific binding N13B2-hVL6 was evaluated as the fold change of fluorescence intensity compared to that of an isotype control on each cell line.

FIGS. 4A,B. Phagocytic indexes variation in presence of increasing concentrations of N13B2-hVL6. (A) Normalized phagocytic index of tumor cells issued from different kinds of Acute Lymphoblastic Leukemia cell lines treated with an antagonist anti-CD127 agent (N13B2-hVL6). (B) Correlation between the phagocytic indexes measured in vitro with the ADCP+/ADCC− anti-CD127 N13B2-hVL6 agent on patient leukemic cells and the fold-change (FC) PDX mouse survival induced by in vivo treatment with this same agent compared to control-treatment.

FIGS. 5A,B. In vivo efficacy of N13B2-hVL6 treatment in a heterogenous cohort of B- and T-ALL patient derived xenografts. (A) Leukemic blast reduction induced by N13B2-hVL6 treatment compared to control treatment in a series of 23 different ALL patient derived xenograft (PDX) mice. (B) PDX identification numbers displayed in A and the corresponding ALL subtypes of patients they derive from.

FIG. 6. Phagocytosis of leukemia cells in a sample treated with N13B2-hVL6 with ADCP+ ADCC− capabilities as compared to a control. Leukemia cells (NALM6 cell line) are colored in red, while human M1 macrophages are colored in green. White arrows point to macrophages phagocytosing leukemia cells.

FIG. 7. Normalized phagocytosis index of leukemia cells in T-ALL models. Two T-ALL cell lineages (HPB-ALL on the right and DND41 IL7-R mutated on the left) have been treated with increasing doses of anti-CD127 antibodies (N13B2-hVL6, 1A11, and EFFI-3-VH3VL3).

FIG. 8. Normalized phagocytosis index of leukemia cells in B-ALL models. Three B-ALL cell lines (697 t(1;19) BCP-ALL on the upper left, NALM6 DUX4 BCP-ALL in the upper right and REH t(12;21) BCP-ALL below) have been treated with increasing doses of anti-CD127 antibodies (N13B2-hVL6, 1A11, HAL and EFFI-3-VH3VL3).

FIG. 9. Toxicity of anti-CD127 antibodies and anti-CD47 antibodies on macrophages. The viability of macrophages treated with an anti-CD127 antibody (N13B2-hVL6) or an anti-CD47 antibody (5F9) has been assessed with increasing doses of antibodies.

FIG. 10. Therapeutic windows for treating ALL with an anti-CD127 antibody or an anti-CD47 antibody. The phagocytic index of normal T cells versus diseased cells (REH model of B-ALL) has been compared in samples treated with increasing doses of an anti-CD127 antibody (N13B2-hVL6) or an anti-CD47 antibody (5F9).

FIGS. 11A,B,C. Lack of in vitro toxicity effect against healthy immune cells. (A) Phagocytosis of macrophages by macrophages (termed here “autophagocytosis” when treated with an anti-CD47 antibody (5F9), an anti-CD127 antibody (N13B2-hVL6) as compared to a negative control antibody (hlgG4). (B). ADCC against human T cells by Natural Killer cells (NK) in presence of an anti-CD127 agent to be used according to the invention (N13B2-hVL6) and a positive control (an anti-CD127 antibody that is known to have an ADCC activity). (C) ADCC against human CD127+ cells by Natural Killer cells (NK) in presence of anti-CD127 agent variants to be used according to the invention (N13B2-h3; N13B2-hVL3; N13B2-hVL4; N13B2-hVLS and N13B2-hVL6) and a positive control (an anti-CD127 antibody that is known to have an ADCC activity). VL3, -VL4, -VL5, VL6 and N13B2-h3 share the same subset of CDR domains (corresponding to HCDR1 of SEQ ID No.: 3; HCDR2 of SEQ ID No.: 4; HCDR3 of SEQ ID No.: 6; LCDR1 of SEQ ID No.: 8; LCDR2 of SEQ ID No.: 10 and LCDR3 of SEQ ID No.: 11) but have different framework sequences. N13B2-hVL6 has the heavy chain variable domain of SEQ ID No. 22, and the light chain variable domain of SEQ ID No. 26; N13B2-hVL3 has the heavy chain variable domain of SEQ ID No. 22, and the light chain variable domain of SEQ ID No. 23; N13B2-hVL4 has the heavy chain variable domain of SEQ ID No. 22, and the light chain variable domain of SEQ ID No. 24; N13B2-hVL5 has the heavy chain variable domain of SEQ ID No. 22, and the light chain variable domain of SEQ ID No. 25.

FIGS. 12A,B. Number of lymphocytes in the blood of healthy volunteers treated with N13B2-hVL6. (A) and (B) correspond to the lymphocyte counts measured in blood samples collected from healthy volunteers participating to the Single Ascending Dose cohort (SAD, 1 intra-venous injection) and to the Multiple Ascending Dose cohort (MAD, 2 intra-venous injections, 15 days apart), respectively.

FIGS. 13A,B. Effect of Dexamethasone on leukemic cells. (A) Example of resistance of T-ALL cells to Dexamathasone (HPB-ALL cell line, 48 h treatment). (B) Induction of CD127 expression in a Dexamethasone dose-dependent manner in HPB-ALL cells (48 h treatment).

FIGS. 14A,B,C. Efficacy of ADCP+/ADCC− anti-CD127 agents in combination with other leukemia treatments. (A) Phagocytosis index of ALL cells in response to N13B2-hVL6 with (red triangles) or without (black dots) Dexamethasone treatment (10 μM for 48 h). (B) Phagocytosis index of NALM6 B-ALL cells in response to anti-CD47 antibody (5F9, Magrolimab) treatment, with the ADCP+/ADCC- anti-CD127 agent N13B2-hVL6 (red triangles) or with isotype control (blue dots). (C) Percentage of survival of 8 mice with overt leukemia ALL PDX treated with a negative control (blue dots), the ADCP+/ADCC− anti-CD127 agent N13B2-hVL6 (orange triangles), ALL standard of care treatment (which consists of administration of a combination of compounds comprising Dexamethasone, Vincristine and PEG-Asparaginase; purple squares), or the combination of N13B2-hVL6 and the standard of care as described here above (a combination of compounds comprising Dexamethasone, Vincristine and PEG-Asparaginase; green inverted triangles) in a cohort of overt leukemia ALL PDX.

FIGS. 15A,B,C. Enhanced in vivo efficacy of N13B2-hVL6 treatment in combination with standard of care treatment in a heterogenous cohort of B- and T-ALL patient derived xenografts. (A) Schematic overview of the in vivo experiment carried out to investigate the efficacy of N13B2-hVL6 in combination with standard of care in ALL (vincristine, PEG-asparaginase and dexamethasone) in a cohort of 6 B-ALL and 3 T-ALL patient derived xenografts (PDXs). (B) Survival curves of 9 PDX mice according to the treatments administered, as indicated, in an overt leukemia setting (treatment started once 1% blasts were detected in the peripheral blood of PDX mice). (C) Percentage of leukemic blasts detectable by flow cytometry in the spleen or the bone marrow of the 4 out of 9 PDX mice treated with standard of care+N13B2-hVL6 surviving throughout the experiment (survival>260 days).

FIGS. 16A,B. Phagocytosis index of leukemia cells treated with several anti-CD127 antibodies as defined in the present description. (A) on BCP-ALL cell line and (B) on REH t(12;21) BCP-ALL cell line. NB13B2-hVL6, VL3, VL4, VL5 and N13B2-h3 share the same subset of CDR domains (corresponding to HCDR1 of SEQ ID No.: 3; HCDR2 of SEQ ID No.: 4; HCDR3 of SEQ ID No.: 6; LCDR1 of SEQ ID No.: 8; LCDR2 of SEQ ID No.: 10 and

LCDR3 of SEQ ID No.: 11) but have different framework sequences. N13B2-hVL6 has the heavy chain variable domain of SEQ ID No. 22, and the light chain variable domain of SEQ ID No. 26; VL3 has the heavy chain variable domain of SEQ ID No. 22, and the light chain variable domain of SEQ ID No. 23; VL4 has the heavy chain variable domain of SEQ ID No. 22, and the light chain variable domain of SEQ ID No. 24; VL5 has the heavy chain variable domain of SEQ ID No. 22, and the light chain variable domain of SEQ ID No. 25. N13B2alpha and beta are chimeric anti-CD127 antibodies which share closely related CDRs domains (with only one or two mutations within the 6 CDRs domains) with N13B2-hVL6 (CDRs of sequences SEQ ID No. 3, SEQ ID No. 4, SEQ ID No. 5, SEQ ID No. 7, SEQ ID No. 9 and SEQ ID No. 11).

FIGS. 17A,B,C. Binding of N13B2-hVL6 and control ADCP+/ADCC+ anti-CD127 antibody to main FCγRs, namely (A) CD16a, (B) CD32a and (C) CD64 assessed by ELISA.

FIGS. 18A,B,C,D,E. Mechanism underlying the antileukemic activity of the ADCP+/ADCC− anti-CD127 N13B2-hVL6 agent on HPB-ALL cell-induced T-ALL. (A) Phagocytic indexes induced by N13B2-hVL6 (red dots) or N13B2-hVL6 LALAPG (green squares) treatment of HPB-ALL T-ALL cells in presence of human macrophages. (B) Percentage of phosphorylated STATS (p-STATS) cells in HPB-ALL cells in response to IL-7 (5ng/mL)+N13B6-hVL6 treatment assessed by flow cytometry. (C) Schematic overview of in vivo interrogation of the HPB-ALL cell line derived xenograft (CDX) model. (D) Median overall survival of HPB-ALL CDX mice in response to treatment with control (blue), with the ADCP+/ADCC− anti-CD127 agent N13B2-hVL6 (orange) or with the ADCP−/ADCC− N13B2-hVL6 LALAPG anti-CD127 agent (green), stars indicate p<0.05 using probing with the Log-rank test. (E) number of viable HPB-ALL cells in response to IL-7 (dark red), IL-7+N13B2-hVL6 (light red), N13B2-hVL6 (light blue) treatments in vitro or in response to mock treatment (dark blue).

FIGS. 19A,B,C,D,E,F,G. Mechanism underlying the antileukemic activity of the ADCP+/ADCC− anti-CD127 N13B2-hVL6 agent on a T-ALL patient derived xenograft. (A) Percentage of leukemic blasts of a T-ALL patient expressing CD127 assessed by flow cytometry. (B) Percentage of phosphorylated STATS (p-STATS) cells with (5 ng/mL) or without IL-7 stimulation and (C) percentage of p-STATS positive cells in the same patient leukemic blasts in response to N13B6-hVL6 dose-response treatment in presence of IL-7 stimulation, assessed by flow cytometry. (D) Antibody Dependent Cellular Phagocytosis measured in response to N13B2-hVL6 (E) Percentage of dead cells and (F) number of viable cells in response to IL-7 (dark red), IL-7 +N13B2-hVL6 (light red), N13B2-hVL6 (light blue) treatments in vitro or in response to mock treatment (dark blue). (G) Median overall survival of the same T-ALL patient derived xenografted mice in response to control (blue) or the ADCP+/ADCC− anti-CD127 agent N13B2-hVL6 (orange).

FIGS. 20A,B,C,D. Mechanism underlying the antileukemic activity of the ADCP+/ADCC− anti-CD127 N13B2-hVL6 agent in NALM6 cell-induced B-ALL. (A) Phagocytic indexes induced by N13B2-hVL6 (red dots) or N13B2-hVL6 LALAPG (green squares) treatment of NALM6 B-ALL cells in presence of human macrophages. (B) Percentage of phosphorylated STATS (p-STATS) cells in NALM6 cells in response to IL-7 (5ng/mL)+N13B6-hVL6 treatment assessed by flow cytometry. (C) Schematic overview of in vivo interrogation of the NALM6 cell line derived xenograft (CDX) model. (D) Median overall survival of NALM6 CDX mice in response to treatment with control (blue), with the ADCP+/ADCC− anti-CD127 agent N13B2-hVL6 (orange) or with the ADCP−/ADCC− N13B2-hVL6 LALAPG anti-CD127 agent (green). Stars indicate p<0.005 using probing with the Log-rank test.

FIGS. 21A,B,C,D. Mechanism underlying the antileukemic activity of the ADCP+/ADCC− anti-CD127 N13B2-hVL6 agent on a B-ALL patient derived xenograft. (A) Percentage of leukemic blasts of a B-ALL patient expressing CD127 assessed by flow cytometry. (B) Percentage of phosphorylated STATS (p-STATS) in presence of IL-7 stimulation and (C) percentage of macrophage mediated phagocytosis in the same patient leukemic blasts, both in response to N13B6-hVL6 dose-response treatment and assessed by flow cytometry. For p-STAT5 assessment (B) the HPB-ALL cell line is used as a positive control. (D) Median overall survival of the same B-ALL patient derived xenografted mice in response to control (blue) or the ADCP+/ADCC− anti-CD127 agent N13B2-hVL6 (orange).

FIGS. 22A,B,C,D. Mechanism underlying the antileukemic activity of the ADCP+/ADCC− anti-CD127 N13B2-hVL6 agent on DND41 cell-induced T-ALL. (A) Phagocytic indexes induced by N13B2-hVL6 (red dots) or N13B2-hVL6 LALAPG (green squares) treatment of DND41 T-ALL cells in presence of human macrophages. (B) Percentage of phosphorylated STATS (p-STATS) cells in DND41 cells in response to IL-7 (5 ng/mL)+N13B6-hVL6 treatment assessed by flow cytometry. (C) Schematic overview of in vivo interrogation of the DND41 cell line derived xenograft (CDX) model. (D) Median overall survival of DND41 CDX mice in response to treatment with control (blue), with the ADCP+/ADCC− anti-CD127 agent N13B2-hVL6 (orange) or with the ADCP−/ADCC− N13B2-hVL6 LALAPG anti-CD127 agent (green). Stars indicate p<0.05 using probing with the Log-rank test.

FIGS. 23A,B,C. ADCP efficacy of ADCP+/ADCC- anti-CD127 antibodies in human mesothelioma. (A) CD127 expression level measured by flow cytometry on human MSTO-211H biphasic malignant mesothelioma cells. (B) Percentage of phagocytosis of human MSTO-211H biphasic mesothelioma cells treated with the ADCP+ ADCC− anti-CD127 antibody N13B2-hVL6. (C) Percentage of phosphorylated STATS (p-STATS) cells in MSTO-211H cells in response to IL-7 (5 ng/mL)+N13B6-hVL6 treatment assessed by flow cytometry, compared to a positive control cell line.

### DETAILED DESCRIPTION OF THE EMBODIMENTS

The present inventors showed for the first time that an anti-CD127 agent as defined herein that has the capability to increase the Antibody Dependent Cellular Phagocytosis (ADCP) activity of macrophages targeting CD127-positive cancer cells but that does not have Antibody Dependent Cytotoxic Activity (ADCC) against CD127-positive cells (in particular against immune cells, more particularly against T cells,) enhances the phagocytosis of tumor cells while sparing healthy lymphocytes and improves the survival rate of leukemia preclinical mouse models. More particularly, the administration of an anti-CD127 agent as defined herein increases the phagocytosis of CD127+ cells, in particular ALL tumor cells, more particularly of T-cell ALL tumor cells and/or B-cell ALL tumor cells and/or malignant mesothelioma cells. More particularly, the inventors illustrated that anti-CD127 antibodies having an IgG4 immunoglobulin domain have the capability to increase the ADCP of CD127-positive tumor cells, while ADCP of normal cells, in particular of normal T cells, is not significantly impacted as compared to the increase in the phagocytosis of tumor cells.

Antibodies kill targeted tumor cells by several mechanisms, including antibody-dependent cell-mediated cytotoxicity (ADCC) and Antibody Dependent Cellular Phagocytosis (ADCP). In antibody-dependent cellular cytotoxicity (ADCC), an Fcγ receptor (FcγR or FCGR) on the surface of an immune effector cell binds to the Fc region of an antibody, which specifically binds to a target cell. Cells that can mediate ADCC are non-specific cytotoxic cells. In contrast, Antibody-dependent cellular phagocytosis (ADCP) relies on phagocytes to devour target cells. ADCP is a highly regulated process in which an antibody eliminates binding target and initiates phagocytosis by linking its Fc domain to a specific Fcy receptor on the phagocytic cell. Unlike ADCC, ADCP can be mediated by monocytes, macrophages, neutrophils and dendritic cells via FcγRIIa (CD32a), FcγRI (CD64) and FcγRIIIa (CD16), where FcγRIIa (CD32a) on macrophages represents the major pathway. These three receptors are able to interact with the Fc portion of antibodies and thus bind to antibodies, thereby inducing ADCC and/or ADCP.

Anti-CD127 agents, in particular anti-CD127 antibodies, more particularly anti-CD127 antagonist antibodies or related compounds, are known to be useful in the treatment of autoimmune diseases and inflammatory diseases by directly interacting with the cells of the immune system of the host (through the capability of said anti-CD127 antibodies to inhibit the IL-7R signaling pathway in T cells). In particular, these anti-CD127 agents are known to be useful in the treatment of autoimmune diseases and inflammatory diseases by inhibiting the survival of antigen-engaged memory T cell survival, without impact on quiescent human T cells (Belarif et al. Nature Communication 2018). Anti-CD127 agents have also been reported to have pre-clinical activity against T-ALL, due to their capacities to inhibit the IL-7R signaling pathway (Akkapeddi, Leukemia, 2018). However, in previous attempts to target IL-7R for treating autoimmune diseases and inflammatory diseases (diabetes and Sjogren syndrome) using anti-CD127 monoclonal antibodies (GSK2618960 and PF-06342674/RN168) due to their capacities to inhibit the IL-7R signaling pathway, clinical developments were stopped and/or complicated by deleterious effects on healthy lymphocytes (Ellis, J. et al. Br. J. Clin. Pharmacol. (2019), Herold, K. C. et al. JCI Insight (2019) and Williams, J. H. et al. AAPS J. (2020)).

The use of anti-CD127 agents having an ADCP capability but not ADCC capability against CD127-positive cells (in particular healthy lymphocytes) for treating CD127-positive cancers by enhancing and/or inducing the phagocytosis of CD127-positive tumor cells by immune cells of the host, in particular by macrophages, was not known or suggested. Further, it is interestingly observed that side effects associated with the administration of an anti-CD127 agent as defined here above (i.e., having an ADCP capability but no ADCC capability against CD127-positive cells (in particular against healthy lymphocytes) to treat a CD127-positive cancer are limited as compared to treatments currently in development, which target CD19-positive cells and leads to healthy B-lymphocyte depletion, such as Blinatumomab. In particular, the inventors illustrate for the first time that anti-CD127 antibodies are able on their own to enhance or initiate the phagocytosis of CD127-positive tumor cells by immune cells of the host, while sparing healthy CD127+ lymphocytes. Indeed, the ADCP+/ADCC− anti-CD127 agent N13B2-hVL6 does not lead to lymphodepletion in healthy volunteers (NCT03980080).

By acting through other mechanism than the disruption of the IL-7/IL-7R interaction, the inventors hereby provide new methods for treating patients, independent of the IL-7R signaling pathway. Such methods of this disclosure are not intuitive and instead are contrary to what has been proposed or suggested in the prior art. For the first time, it is disclosed that cancers, including solid cancer and liquid cancers, in particular ALL, can be treated by inducing the phagocytosis of tumoral cells by administering an anti-CD127 agent that has an ADCP capability against CD127-positive tumor cells but no ADCC capability against CD127+cells, and not by inhibiting or disrupting the IL-7/IL-7R interaction. This lead to a new clinical situation, allowing effective treatment of patients who have for example a dysfunctional IL-7R signaling pathway, which is a clinical situation frequently encountered.

It is illustrated for the first time that anti-CD127 antibodies, in particular anti-CD127-IgG4 antibodies, are able to increase on their own the phagocytosis of CD127-positive tumor cells by the ADCP mechanism, without the need of other therapeutic agents.

To sum up, the present invention of using an anti-CD127 agent that has an ADCP capability against CD127-positive tumor cells but no ADCC capability against CD127+ cells (including immune cells) or the method comprising the administration of such an agent to treat a patient having CD127-positive tumor cells has the following advantages:
it lacks cytotoxicity against immune cells of the patient who is treated with such an agent, in particular it does not reduce the number of macrophages and/or healthy lymphocytes of the patient;
it can target patients having a tumor wherein the IL-7R signaling pathway is dysfunctional (e.g., the IL-7R signaling pathway cannot be enhanced, silenced, activated or inhibited as compared to healthy cells), a situation often met in the clinic (Zenatti P P, et al. Nat. Genet. 2011; Shochat C, et al. Exp. Med. 2011; Roberts K G, et al. Cancer Cell. 2012; Geron et al. Nature Communication 2022)
it enhances the survival rate and the survival length.

It was not obvious to treat patients having a cancer, in particular leukemia, and more particularly ALL, with tumor cells expressing CD127 with an anti-CD127 agent that has an ADCP capability but no ADCC capability against CD127-positive tumor cells. In particular, it was not obvious to administer an anti-CD127 agent (e.g., an agent used in the prior art to inhibit the IL-7R signaling pathway induced by IL-7) to a patient having a tumor wherein cells resist to IL-7R-signaling pathway blockade (because the pathway is either constitutively activated or cannot be activated). It corresponds to many clinical situations (Zenatti P P, et al. Nat. Genet. 2011; Shochat C, et al. J Exp. Med. 2011; Roberts K G, et al. Cancer Cell. 2012; Geron et al., Nature Communication 2022). For example, IL7R mutated ALLs with constitutive activation of the IL-7R signaling pathway are highly prevalent in T-ALL. The properties of the anti-CD127 agents of the invention, namely their ADCP capability and lack of ADCC capability against CD127+cells, allows the treatment of such a category of patients through the enhancement of the phagocytosis of tumor cells, independently of their activity on the IL-7R signaling pathway.

The inventors surprisingly observed that anti-CD127 agent that has an ADCP capability but no ADCC capability against CD127-positive cells (including immune and tumor cells), like the antibody referenced N13B2-hVL6 in the working examples of the invention, had an antitumoral activity against leukemias and solid tumors. N13B2-hVL6 is an antagonist monoclonal antibody directed against CD127 (Belarif et al., Nature Communication 2018) and as such was hypothesized to mediate its antileukemic effect primarily due to its capacity to efficiently block the pro-survival and pro-proliferation signaling cascade mediated by the IL-7/IL-7R pathway. Surprisingly, the inventors found that the antitumoral effect including against leukemias and solid tumors of N13B2-hVL6 was consistently mediated by its capacity to induce ADCP, a mechanism which has not yet been reported for anti-IL-7R antibodies for the treatment of cancer (including ALL). Due to this robust ADCP induction, cancer, including leukemias, with CD127 expression, in particular high CD127 expression, regardless of their functional dependency on the IL-7R pathway are predicted to positively respond to a treatment with an anti-CD127 agent as defined here above, thereby opening therapeutic opportunities for any patient with sufficient expression of CD127 at the surface of their tumor cells, in particular when these tumor cells are associated to leukemia, more particularly to ALL. Of importance, this includes IL-7R mutated ALLs with constitutive activation of the pathway, which are highly prevalent in T-ALL, such as the DND41 cell line. It was previously predicted to be refractory to IL-7R targeting by N13B2-hVL6 since constitutive activation mutations of IL7R cannot be antagonized by this type of antibodies.

Further, the inventors surprisingly found that the anti-CD127 agents lacking ADCC capabilities had strong ADCP capability and are effective on CD127 positive tumor cells. ADCC and ADCP both rely on the binding to FcyRs. For example, N13B2-hVL6 is an IgG4 antibody which lacks ADCC capacities, as do most IgG4 antibodies, since IgG4 formats have weak affinities for all FC receptors except FcyRI and are therefore poor inducers of Fc-mediated effector functions (Yu et al. Journal of Hematology & Oncology, Tay et al. Front Immunol 2019). An antibody that lacks ADCC capacity is expected to be unable to induce other Fc-mediated effector functions such as ADCP, as all Fc-mediated effector mechanisms rely on the binding of the Fc portion of antibodies to Fc gamma receptors. As such, providing anti-CD127 agents with the capacity to induce high levels of ADCP without inducing a high level of ADCC is highly unexpected.

The inventors were also very surprised to find that anti-CD127 IgG4 antibodies, N13B2-hVL6 for example, has the capability to induce ADCP. IgG4 antibodies usually lack Fc-mediated effector functions such as ADCP. IgG4 format antibodies have weak affinities for all FC receptors except FcyRI and are therefore poor inducers of Fc-mediated effector functions (Yu et al. Journal of Hematology & Oncology, Tay et al. Front Immunol 2019). Illustratively, all currently approved anti-PD-1 antibodies are in the IgG4 format to avoid Fc-mediated elimination of PD1+ CD8+ T lymphocytes. Conversely, antibodies directed against CTLA-4 (for instance Ipilimumab), which is expressed by immunosuppressive Treg cells in the tumor microenvironment (TME), are used in the clinic in IgG1 format to promote elimination of Tregs via ADCC/ADCP mechanisms (Du et al., Cell Res 2018). Surprisingly, although N13B2-hVL6 is an IgG4 antibody, the inventors found that it was able to induce strong levels of ADCP that can be superior to that of reference pro-phagocytic anti-CD47 antibodies (for example in CD127-high leukemia cell lines such as REH). Also unexpectedly, N13B2-hVL6 displays superior ADCP capacities compared to all other IgG1 format anti-CD127 antibodies (1A11, HAL and Effi3-VH3VL3) in all tested leukemia cell lines.

Moreover, the inventors also illustrate that the anti-CD127 agent to be used in the method of the invention, or for use according to the invention, do not have or have a non-significant effect on ADCP capability on normal T cells, as compared to other therapeutic compounds usually administered in the treatment of cancers, in particular leukemia, and more particularly T-cell and B-cell leukemia. Thus, the use of the anti-CD127 agent according to the method of the invention leads to an increase in the phagocytosis of CD127-positive tumor cells, while it does not deplete the normal T cell population of the host, thereby reducing side effects usually associated with multiple antibody-related treatment of cancers. In particular, the inventors show for the first time that administering an anti-CD127 agent as defined herein to healthy humans does not lead to lymphodepletion. Further, no adverse effects were observed in these healthy volunteers, on the contrary to observation in humans receiving the drugs currently administered which induce severe adverse effects, like anti CD3- anti-CD19 bispecific agents (see Condo-Royo et al., Drugs Context 2020).

In one aspect, the present invention relates to an anti-CD127 agent, in particular an anti-CD127 antibody or antigen-binding fragment thereof or antigen-binding antibody mimetic, for use as a medicament in the treatment of a patient having a CD127-positive cancer, in particular by enhancing the phagocytosis of CD127-positive tumor cells, more particularly by macrophages, wherein the anti-CD127 agent has Antibody Dependent Cellular Phagocytosis (ADCP) activity against CD127-positive tumor cells, in particular by macrophages, and does not have Antibody Dependent Cytotoxic Activity (ADCC), in particular against immune cells, more particularly against T cells.

In another aspect, the present invention relates to an anti-CD127 agent for use in the treatment of a patient having a CD127-positive cancer, particularly by phagocytosis of CD127-positive tumor cells, in particular by macrophages, in particular a CD127-positive leukemia or a CD127-positive solid cancer, more particularly a CD127-positive ALL, even more particularly a CD127-positive T-cell ALL or a B-cell ALL.

In another aspect, the present invention relates to a method for treating a patient having a CD127-positive cancer, in particular a CD127-positive leukemia or a CD127-positive solid cancer, more particularly a patient having a CD127-positive T-cell ALL or a B-cell ALL, by increasing the phagocytosis of CD127-positive tumor cells, in particular by macrophages of the patient.

The present invention also relates to the use of an anti-CD127 agent for the manufacture of a medicament for the treatment of cancer, in particular leukemia or solid cancer, by enhancing the phagocytosis of CD127-positive tumor cells.

In some embodiments, a therapeutically effective amount of the anti-CD127 agent for use in the method of the invention or for use according to the invention is administered to a subject having a cancer.

The present invention further relates to anti-CD127 agent having (i.e. increasing as compared to a negative control which can be an isotype control such as MOTA-hIgG4) Antibody Dependent Cellular Phagocytosis (ADCP) activity of macrophages targeting CD127-positive cancer cells, in particular wherein said activity is achieved by or involves macrophages, for the treatment of a patient having a CD127-positive cancer, in particular a patient having a CD127-positive leukemia, more particularly having a CD127-positive ALL or in particular a patient having a a CD127-positive solid tumor, more particularly having a CD127-positive malignant mesothelioma.

The present invention also relates to anti-CD127 antibodies or antigen-binding fragments thereof, having (i.e. increasing as compared to a negative control) Antibody Dependent Cellular Phagocytosis (ADCP) activity of macrophages targeting CD127-positive cancer cells, in particular wherein said activity is achieved by or involves macrophages, for the treatment of a patient having an ALL selected from the group consisting of CD127 overexpressing ALL (an overexpressing ALL may be determined by comparing CD127 expression in ALL cells as compared to CD127 expression in healthy bone marrow), CD127 and/or JAK-STAT pathway mutated ALL (as opposed to healthy cells), including BCR-ABL1—like ALL, as well as B cell precursor ALL bearing the following cytogenetics: t(1;19), t(12;21), MLL-rearrangements, Hyperdiploid karyotypes, trisomy 4 and trisomy 10. In a particular embodiment, the invention relates to anti-CD127 antibodies or antigen-binding fragments thereof, having (i.e. increasing as compared to a negative control) Antibody Dependent Cellular Phagocytosis (ADCP) activity on macrophages targeting CD127-positive cancer cells, in particular wherein said activity is achieved by or involves macrophages, for the treatment of ALL selected from the group consisting of CD127 wild type T-ALL (HPB-ALL cell line), CD127-mutated T-ALL (DND41 cell line), t(1;19) B-ALL (697 cell line), t(12;21) B-ALL (REH cell line) and t(5;12) B-ALL (NALM6 cell line).

In another aspect, the invention relates to a pharmaceutical composition, suitable for administration to a mammalian host, in particular a human host, comprising an anti-CD127 agent as defined herein, and a pharmaceutical vehicle.

In particular, the invention relates to the pharmaceutical composition for use in the treatment of a patient, in particular a human patient having a CD127-positive cancer, the composition comprising an anti-CD127 agent that increases the phagocytosis of CD127-positive tumor cells and increasing (as compared to a negative control) Antibody Dependent Cellular Phagocytosis (ADCP) activity of macrophages targeting CD127-positive cancer cells and a pharmaceutical vehicle.

The present invention also relates to a combination of therapeutic agents for treating a patient having a CD127-positive cancer, in particular in leukemia, more particularly ALL, in particular in solid tumors, more particularly malignant mesothelioma, said combination comprising an anti-CD127 agent which increases the phagocytosis of CD127-positive tumor cells, and having (i.e. increasing as compared to a negative control) Antibody Dependent Cellular Phagocytosis (ADCP) activity of macrophages targeting CD127-positive cancer cells, the combination further comprising at least a second (i.e. distinct) therapeutic agent.

In a particular embodiment, the invention relates to the use of anti-CD127 agents as defined herein in order to deplete subpopulations of CD127-positive tumor cells, in particular CD127-positive T- or B-ALL cells or CD127-positive mesothelioma cells in a patient having a cancer, in particular a cancer selected in the group of cancers specified herein, by enhancing the phagocytosis of CD127-positive tumor cells, in particular by macrophages.

The invention also relates to an anti-CD127 agent for use as a medicament for promoting (enhancing and/or inducing) phagocytosis of CD127-positive cells of a patient, in particular CD127-positive tumor cells, particularly by macrophages, the anti-CD127 agent having Antibody Dependent Cellular Phagocytosis (ADCP) activity against CD127-positive tumor cells, in particular by macrophages, and the anti-CD127 agent not having Antibody Dependent Cytotoxic Activity (ADCC), in particular against immune cells, more particularly against T cells.

The invention also relates to the use of an anti-CD127 agent for preparing a medicament for promoting (enhancing and/or inducing) phagocytosis of CD127-positive cells of a patient, in particular CD127-positive tumor cells, particularly by macrophages, the anti-CD127 agent having Antibody Dependent Cellular Phagocytosis (ADCP) activity against CD127-positive tumor cells, in particular by macrophages, and the anti-CD127 agent not having Antibody Dependent Cytotoxic Activity (ADCC), in particular against immune cells, more particularly against T cells.

Definitions

As used herein, “antibody” includes polyclonal, monoclonal, recombinant, chimeric, humanized, bispecific, multispecific and modified antibodies, as well as monovalent and divalent antigen-binding fragments thereof. Furthermore, “antibody” includes synthetic antibodies, single chain antibodies, and fragments thereof. The antibody may be a human or nonhuman antibody. A nonhuman antibody may be humanized by recombinant methods to reduce its immunogenicity in human. More specifically the term “antibody” refers to a monoclonal antibody or recombinant monoclonal antibodies, or an antigen-binding fragment thereof.

As used herein, a “monoclonal antibody” is intended to refer to a preparation of antibody molecules, wherein antibodies share a common heavy chain and common light chain amino acid sequence, in contrast with “polyclonal” antibody preparations which contain a mixture of antibodies of different amino acid sequences. Monoclonal antibodies can be generated by several known technologies like phage, bacteria, yeast or ribosomal display, as well as by classical methods exemplified by hybridoma-derived antibodies. Thus, the term “monoclonal” is used to refer to all antibodies derived from one nucleic acid clone.

As used herein, an “antigen-binding fragment of an antibody” means a part of an antibody, i.e., a molecule corresponding to a portion of the structure of the antibody of the invention, that exhibits antigen-binding capacity for CD127, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragments that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. For illustration purpose of specific embodiments of the invention, antigen binding fragments of an antibody that contain the variable domains comprising the CDRs of said antibody encompass Fv, dsFv, scFv, Fab, Fab′, F(ab′)2.

Antibodies and antigen-binding fragments of antibodies comprise at least a light chain variable domain and a heavy chain variable domain, each one comprising three hypervariable domains designated CDRs (Complementary Determining Regions). These domains encompass the recognition site for the antigen, i.e., CD127, in particular human CD127, and most particularly the extracellular domain of human CD127, thereby defining antigen recognition specificity.

Each Light and Heavy chain variable domain (respectively VL and VH) has three CDRs, designated VL-CDR1 (or LCDR1), VL-CDR2 (or LCDR2), VL-CDR3 (or LCDR3) and VH-CDR1 (or HCDR1), VH-CDR2 (or HCDR2), VH-CDR3 (or HCDR3), respectively.

Antibodies and antigen-binding fragments thereof may comprise or derive from any of the commonly known immunoglobulin classes, including but not limited to IgA, secretory IgA, IgE, IgG and IgM. IgG subclasses are also well known to those in the art and include but are not limited to human IgGl, IgG2, IgG3 and IgG4.

Antigen-binding antibody mimetics are organic compounds that specifically bind antigens, but that are not structurally related to antibodies. They are usually artificial peptides or small proteins with a molar mass of about 3 to 20 kDa. Nucleic acids and small molecules are sometimes considered antibody mimetics as well, but not artificial antibodies, antibody fragments and fusion proteins composed from these. Common advantages over antibodies are better solubility, tissue penetration, stability towards heat and enzymes, and comparatively low production costs. Antibody mimetics are being developed as therapeutic and diagnostic agents. Antigen-binding antibody mimetics may also be selected among the group comprising affibodies, affilins, affimers, affitins, DARPins, and Monobodies.

As used herein, the term “specifically binds to” or “binds specifically” refers to the capability of anti-CD127 agent to be used in the method of the invention or for use according to the invention to interact with CD127 and to bind with CD127, preferably human CD127, while they do not bind or they bind with a significantly weaker binding affinity to other molecules, in particular to other proteins. Binding and binding specificity can be assayed by SPR (Surface Plasmon Resonance e.g., Biacore), ELISA or Western Blot analysis. In a particular embodiment, the ability of the anti-CD127 agent to bind to CD127 is considered to be specific when the binding affinity is of at least about 1×10−6 M, 1×10−7 M, 1×10−8 M, 1×10−9 M, 1×10−10 M, 1×10−11 M, 1×10−12 M, or more, and/or bind to a target with an affinity that is at least two-fold greater than its affinity for a nonspecific protein.

As used herein, the term “CD127” relates to a CD127 from a mammal species, preferably a human CD127, and most preferably human CD127 of SEQ ID No. 1. CD127, also known as Interleukin-7 receptor subunit alpha (IL7R-a), is a protein that in humans is encoded by the IL7R gene. CD127 is a type I cytokine receptor and is a subunit of the functional Interleukin-7 receptor and Thymic Stromal Lymphopoietin (TSLP) receptors. CD127 may correspond to the protein referenced under NCBI Sequence No. NP_002176.2. Alternatively, CD127 may correspond to a protein having the amino acid sequence of SEQ ID No. 1. The extracellular domain of CD127, which is likely to be recognized and bound to by anti-CD127 agent used in the invention may correspond to the amino acid sequence of SEQ ID No. 2.

The terms “cancer” and “tumor” have their general meaning in the art and refers to a group of diseases involving abnormal cell growth with the potential to invade or spread to other parts of the body. The term “cancer” further encompasses both primary and metastatic cancers.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread of the disease, preventing or delaying the recurrence of the disease, delaying or slowing the progression of the disease, ameliorating the disease state, providing a remission (partial or total) of the disease, enabling to decrease the administered dose of one or more other medications required or used to treat the disease, increasing the quality of life, and/or prolonging survival, preventing or alleviating side-effects of chemotherapeutic treatment, or novel treatments of ALL, such as the severe cytokine release syndrome and neurotoxic side effects of the treatments comprising administration of anti-CD3 anti-CD19 bi-specific antibodies.

As used herein the term “antibody-dependent cell-mediated phagocytosis” or ‘ADCP’, refers to a cell-mediated reaction in which phagocytes, in particular macrophages, recognize bound antibody on a target cell and subsequently engulf the target cells, leading to their digestion within phagosomes. An anti-CD127 agent may be considered to have an ADCP capacity against CD127-positive tumor cells, in particular by macrophages, when it is able to induce a phagocytic score of strictly over 1. The phagocytic score is the fold change of percentage of CPD/CTG double positive cells within CTG positive cells (macrophages) as compared to a relevant isotype control, multiplied by the fold change of geometric mean of CPD fluorescence within CTG positive cells (macrophages) as compared to a relevant isotype control.

An anti-CD127 agent may be considered to have an ADCP capacity against CD127-positive tumor cells, in particular by macrophages, when it is able to increase the phagocytosis of CD127-positive tumor cells, in particular by macrophages, by at least 10%, preferably at least 15%, more preferably at least 20%, even more preferably at least 30%, and most preferably at least 40%, as compared to a control experiment (e.g. the same experiment conducted in absence of the anti-CD127 agent or in presence of an isotype control).

As used herein the term “Antibody-Dependent Cellular Cytotoxicity” or ‘ADCC’, refers to an immune mechanism through which Fc receptor-bearing effector cells can recognize and kill antibody-coated target cells expressing antigens recognized antibodies on their surface. More particularly, ADCC may refer the binding of an anti-CD127 agent (e.g., an antibody) to an epitope expressed on target cells and the subsequent Fc-dependent recruitment of effector immune cells expressing Fc receptors (essentially NK cells and activated lymphocytes), resulting in the killing of target cells mainly by granzyme/perforin-based mechanisms. An anti-CD127 agent that does not have an ADCC activity, in particular against immune cells, in particular against T cell, may be attributed to an agent (e.g., an antibody) which is able to induce a specific ADCC score strictly below 500 cpm using human NK cells as effector cells and radioactive chrome (Cr51) labeling to measure cell lysis. The ADCC assay may be carried out by co-culture of ⁵¹Cr labelled T cells (target cells) with human NK cells (effector cells) at a ratio of 10 NK for 1 T cell. Cytotoxicity (ADCC) may be evaluated by measurement of radioactive counts per minute (cpm) in the supernatant of this coculture after 4 hours of incubation at 37° C., 5% CO2.

As used herein, the term IL-7 signaling pathway is related to the intracellular molecular pathway induced when IL-7 binds to IL-7R and triggers the activation of several signaling pathways, including the Janus kinases (JAK) -1 and -3, signal transducer and activator of transcription 5 (STAT5) and phosphatidylinositol 3-kinase (PI3-k). The IL-7 signaling pathway may be considered to be activated when the phosphorylation of PI3-k and/or STAT5 and/or ERK is increased as compared to a negative control wherein the IL-7R is not stimulated (for example in absence of IL-7).
Anti-CD127 Agents to be Used in the Method of the Invention or for Use According to the Invention

As used herein, an anti-CD127 agent refers to a compound selected from the list consisting of antibodies; antigen-binding fragments of an antibody; antigen-binding antibody mimetics; macromolecules comprising an antibody, an antigen-binding fragments of an antibody, or an antigen-binding antibody mimetics; and which binds, in particular specifically binds, to CD127, in particular human CD127, in particular human CD127 of SEQ ID No. 1, in particular to the extracellular domain of (human) CD127, most particularly to the extracellular domain of human CD127 of SEQ ID No. 2. The anti-CD127 agent to be used in the method of the invention, or for use according to the invention, has furthermore the capability to increase the Antibody Dependent Cellular Phagocytosis (ADCP) activity against CD127-positive tumor cells by macrophages, and does not have Antibody Dependent Cellular Cytotoxic (ADCC) activity, in particular against immune cells. In a particular embodiment of the invention, the anti-CD127 agent to be used in the method of the invention, or for use according to the invention, has the capability to increase the Antibody Dependent Cellular Phagocytosis (ADCP) activity against CD127-positive tumor cells by macrophages, and preferably does not have Antibody Dependent Cellular Cytotoxic (ADCC) activity, in particular against immune cells

An increase in the phagocytosis of CD127-positive tumor cells, in particular by macrophages, may be assessed by a comparison of the phagocytosis of CD127-positive tumor cells in two experiments, one experiment in presence of an anti-CD127 agent and one experiment in absence of the anti-CD127 agent. An increase in the phagocytosis of CD127-positive tumor cells, in particular by macrophages as compared to the same experiment in absence of the anti-CD127 agent may be considered when the phagocytosis is raised by at least 10%, preferably at least 15%, more preferably at least 20%, even more preferably at least 30%; and most preferably at least 40%, as compared to the control.

The anti-CD127 agent used in the method of the invention of for use according to the invention shows an increased Antibody-Dependant Cellular Phagocytosis (ADCP) activity against CD127-positive cells, in particular CD127-positive tumor cells, in particular by macrophages. Antibody ADCP increase may be considered positive when specific phagocytosis against CD127-positive cells is superior by 10% in presence of the anti-CD127 agent as compared to the phagocytosis of the same CD127-positive cells in absence of the anti-CD127 agent or in presence of an isotype control. ADCP properties can be evaluated in an ADCP assay such as the test disclosed in the examples of the invention. More particularly, an ADCP assay may comprise the following steps: co-culturing for one hour leukemic cells labeled with a fluorescent dye and in presence of the anti-CD127 agent or in absence of the anti-CD127 agent with phagocytic cells, in particular macrophages, labeled with another fluorescent dye, and measuring the fluorescence of leukemic cells within phagocytic cells. The ADCP assay is preferably carried out in presence of human macrophages.

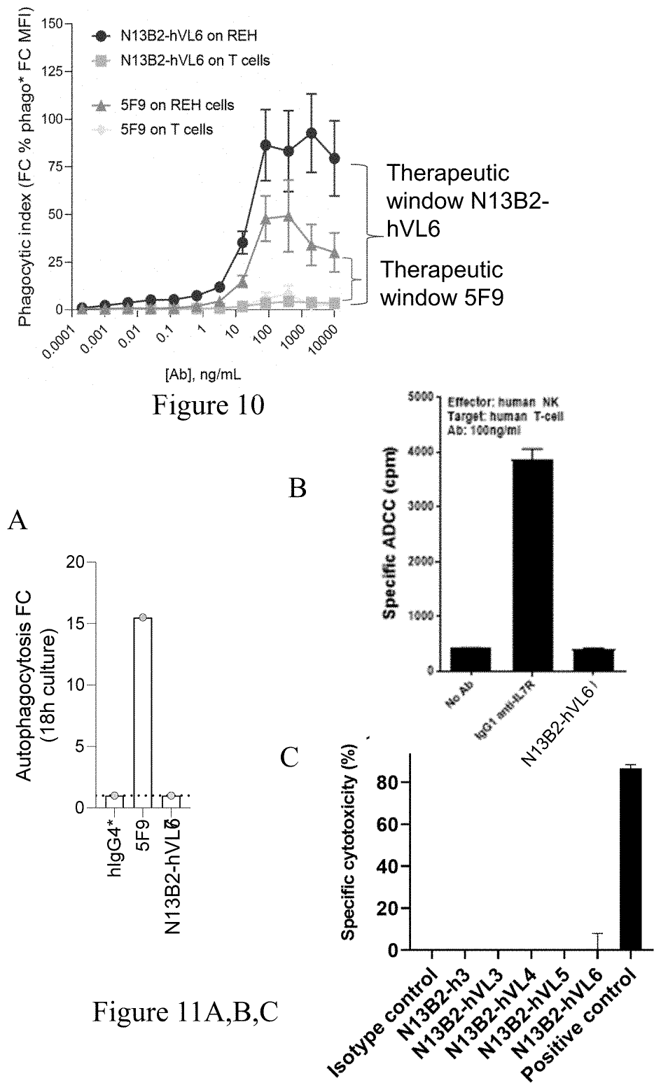

The anti-CD127 agent of the invention does not induce ADCC, in particular against immune cells, more particularly against T cells and/or of tumor cells. In particular, the ADCC potential of an anti-CD127 agent to be used according to the invention may be assessed according to the method disclosed here above or according to the examples of the invention, more particularly according to the method used in the example illustrated in FIGS. 10B and 10Cof the invention.

In a particular embodiment of the invention, the anti-CD127 agent is an antibody or a related compound, like but not limited to antigen-binding antibody fragment, and antigen-binding antibody mimetic, and has an enhanced antibody-dependent cell-mediated phagocytosis (ADCP) activity against CD127-positive cells and no Antibody-Dependent Cellular Cytotoxicity (ADCC), as compared to another antibody or related compound which does not increase ADCP against CD127-positive cells and/or increase ADCC activities, in particular against immune cells, more particularly against T cells , including some anti-CD127 antibodies that are able to recognize and bind to CD127, but do not have any enhancement capability on the antibody-dependent cell-mediated phagocytosis of CD127-positive tumor cells.

In a particular embodiment of the invention, the anti-CD127 agent is an anti-CD127 antibody, or an antigen-binding fragment thereof, that is a humanized antibody, and which comprises constant domains derived from human constant domains of antibodies.

In a particular embodiment of the invention, the anti-CD127 agent is selected from the group consisting of chimeric antibodies, humanized antibodies and fully human monoclonal antibodies.

In a particular embodiment of the invention, the anti-CD127 agent is an anti-CD127 antigen-binding fragment of an antibody that shares the same functions as a full anti-CD127 agent according to the invention that is an antibody, and has inferior or equal to 500, 400, 300, 200, 100 or 50 amino acids and have at least the capacity to bind CD127, the capacity to induce ADCP, and has not ADCC activity. In particular, an anti-CD127 antigen-binding fragment of an antibody according to the invention have a size from 80 to 200, in particular from 100 to 200, in particular from 80 to 160, in particular from 100 to 160 amino acids, and have at least the capacity to bind CD127.

In a particular embodiment of the invention, the anti-CD127 agent of the invention is a functional fragment of an anti-CD127 antibody. Functional equivalents of such an antibody include but are not limited to molecules that bind to CD127, with the proviso that these functional fragments have an ADCP capability and do not have ADCC capability. A suitable functional fragment might comprise, for example, a truncated form of a full antibody. Particularly, the functional equivalent consists of an amino acid sequence having at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity with any anti-CD127 antibody or antigen-binding fragment thereof disclosed in the present description over their entire length. As used herein, the term “full antibody” refers to an antibody that is an anti-CD127 agent according to the invention for which the functional equivalent of the invention has similar function. The percentages of identity to which reference is made in the presentation of the present invention are determined on the basis of a global alignment of sequences to be compared, that is to say, on an alignment of sequences over their entire length, using for example the algorithm of Needleman and Wunsch 1970. This sequence comparison can be done for example using the needle software by using the parameter “Gap open” equal to 10.0, the parameter “Gap Extend” equal to 0.5, and a matrix “BLOSUM 62”. Software such as needle is available on the website ebi.ac.uk worldwide, under the name “needle”. Accordingly, the present invention provides a polypeptide, in particular a functional fragment of an antibody, which is an antagonist of CD127, which has antibody-dependent cell-mediated phagocytosis (ADCP) activity against CD127-positive cells, in particular by macrophages, and no Antibody-Depdendent Cellular Cytotoxicity (ADCC), in particular against immune cells, more particularly on T cells, said polypeptide comprises consecutive amino acids having a sequence which has at least at least 80% identity, more particularly at least 85%, at least 86%, at least 87%, at least 88%, at least 89%, at least 90%, at least 91%, at least 92%, at least 93%, at least 94%, at least 95%, at least 96%, at least 97%, at least 98% and even more particularly at least 99% of identity with antibody N13B2hVL6 constituted of the heavy chain of SEQ ID No. 27 and the light chain of SEQ ID No. 28.

In a particular embodiment, the antibody light chain constant domain is derived from a human kappa light chain constant domain.

More particularly, the antibody heavy chain constant domain is derived from a human IgG1, IgG2, IgG3, or IgG4 heavy chain constant region, particularly from IgG4 heavy chain constant region. “Derived from” means encompassing some punctual mutations by amino acid substitutions such as IgG4 (S228P) or IgGl(E333A) (see Yang and Ambrogelly, Current Opinion in Biotechnology 2014 and Okasaki et al., J Mol Biol 2004). These mutations well known from the skilled person in the art, generally modify some parent chain properties. For example, they lead to less immunogenicity compared to the parental antibody or abrogate FcγReceptor binding or avoid dimerization of the monomer antibody or stabilize the dimerization rendering antibodies better for human therapeutic uses.

In a particular embodiment of the invention, the anti-CD127 agent to be used according to the method of the invention or for use according to the invention increases Antibody-Dependent Cellular Phagocytosis (ADCP) of CD127-positive cells, in particular CD127-positive tumor cells, in particular by macrophages, and thereof increases the phagocytosis of CD127-cancer cells in the host, the anti CD127 agent being an antibody or an antigen-binding fragment thereof which belongs to the class of IgG4 mammalian immunoglobulins, and has no ADCC activity. In a more particular embodiment of the invention, the anti-CD127 agent is an antibody or an antigen-binding fragment thereof, belongs to the class of IgG4 mammalian immunoglobulins.

In a particular embodiment of the invention, the anti-CD127 agent is an antagonist of IL-7R signaling pathway induced by the binding of IL-7 to CD127. In other words, the anti-CD127 agent used in the method of the invention or for use according to the invention has the capability to disrupt or block the binding between IL-7 and CD127, as compared to the binding between IL-7 and CD127 in absence of the anti-CD127 agent. As used herein, a CD127 antagonist agent, in particular an anti-CD127 antagonist antibody or related compound, has its general meaning in the art and refers to any compound, natural or synthetic, that blocks, suppresses, or reduces the biological activity of IL-7. In particular, the CD127 antagonist inhibits the interactions between IL-7 and CD127. In particular, the CD127 antagonist inhibits or reduces the activation of the phosphatidylinositol 3-kinase and/or the ERK signaling pathway induced by IL-7. In the invention, it can be considered that an antibody (or antigen-binding fragment thereof) reduces, inhibits or blocks the binding of IL-7 to CD127 if said antibody (or antigen-binding fragment thereof) induces an increase superior to 1 log, preferably superior to 2 log, more preferably superior to 3 log, most preferably superior to 4 log, of the KD value of IL-7 to CD127 in a binding competitive assay by Blitz, as compared to the KD value of IL-7 to CD127 in presence of a control antibody (i.e. an antibody which does not specifically bind to IL-7 nor CD127).

In a particular embodiment, the anti-CD127 agent does not induce the activation of the phosphatidylinositol 3-kinase and/or the ERK signaling pathway and/or does not induce the phosphorylation of STAT5, particularly does not induce the activation of the phosphatidylinositol 3-kinase and the ERK signaling pathway and does not induce the phosphorylation of STAT5.

In a particular embodiment, the anti-CD127 agent does not induce lymphodepletion in the patient, particularly does not lead to lymphodepletion in the patient. Lymphodepletion corresponds to a reduction in the overall number of lymphocytes in the patient. An anti-CD127 agent may be considered not to induce lymphodepletion, when it is presence, the overall number of lymphocytes in a biological sample issued from the patient, is not lower than at least 50%, preferably at least 40%, more preferably at least 30%, even more preferably at least 20%, and most preferably at least 10%, as compared to a control sample obtained from the patient (e.g. a sample obtained from the same patient before administration of the anti-CD127 agent) or as compared to usual numbers of lymphocytes in a healthy human, which are known by skilled artisans. Lymphodepletion may be assessed according to the method disclosed in the present application, for example by the method illustrated on FIG. 11. In a particular embodiment of the invention, the anti-CD127 agent is an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 5 or SEQ ID No. 6;
   and a VL chain comprising at least the following amino acid sequences:
VLCDR1 SEQ ID No. 7 or SEQ ID No. 8;
VLCDR2 SEQ ID No. 9 or SEQ ID No. 10;
VLCDR3 SEQ ID No. 11,
   said anti-CD127 antibody or an antigen-binding fragment thereof exhibiting ADCP activity against CD127-positive cells, in particular CD127-positive tumor cells. In an embodiment, the anti-human CD127 antibody or an antigen-binding fragment enhances the phagocytosis on CD127-positive cells, in particular CD127-positive tumor cells, by macrophages when administered to a patient, and has no ADCC activity, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

CDR domains have been identified according to the KABAT numbering.

In a particular embodiment, the anti-CD127 agent is an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 5;
   and a VL chain comprising at least the following amino acid sequences:
VLCDR1 SEQ ID No. 7;
VLCDR2 SEQ ID No. 9;
VLCDR3 SEQ ID No. 11.

In a particular embodiment of the invention, the anti-CD127 agent is an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 6;
   and a VL chain comprising at least the following amino acid sequences:
VLCDR1 SEQ ID No. 8;
VLCDR2 SEQ ID No. 10;
VLCDR3 SEQ ID No. 11.

In a particular aspect of the invention, the anti-CD127 agent is an anti-human CD127 antagonist antibody or an antigen-binding fragment thereof which comprises:

a heavy chain variable domain comprising or consisting of the amino acid sequence set forth in SEQ ID No. 12, SEQ ID No. 13, SEQ ID No. 14, SEQ ID No. 15 or SEQ ID No. 22, in particular SEQ ID No. 15 or SEQ ID No. 22; and

a light chain variable domain comprising or consisting of the amino acid sequence set forth in SEQ ID No. 16, SEQ ID No. 17, SEQ ID No. 18, SEQ ID No. 19, SEQ ID No. 23, SEQ ID No. 24, SEQ ID No. 25 or SEQ ID No. 26, in particular SEQ ID No. 19 or SEQ ID No. 26,

said anti-CD127 antibody or an antigen-binding fragment thereof exhibiting ADCP activity against CD127-positive cells, in particular CD127-positive tumor cells, and has no ADCC activity. In an embodiment, the anti-human CD127 antibody or an antigen-binding fragment enhances the phagocytosis of CD127-positive cells, in particular CD127-positive tumor cells, by macrophages when administered to a patient, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

In a particular embodiment, the heavy chain variable domain is linked to the constant heavy chain consisting of the sequence of SEQ ID No: 30, to constitute a complete antibody heavy chain.

In a particular embodiment, the light chain variable domain is linked to the constant light chain consisting of a sequence selected from SEQ ID No: 31 and SEQ ID No: 32, in particular SEQ ID No: 31, to constitute a complete antibody light chain.

In a particular embodiment, the anti-CD127 agent is an anti-human CD127 antagonist antibody or an antigen-binding fragment thereof which comprises the heavy chain variable domain comprising or consisting of the amino acid sequence set forth in SEQ ID No. 12, and the light chain variable domain comprising or consisting of the amino acid sequence set forth in SEQ ID No. 16.

In a particular embodiment, the anti-CD127 agent is an anti-human CD127 antagonist antibody or an antigen-binding fragment thereof which comprises the heavy chain variable domain comprising or consisting of the amino acid sequence set forth in SEQ ID No. 15, and the light chain variable domain comprising or consisting of the amino acid sequence set forth in SEQ ID No. 19.

In a particular embodiment, the anti-CD127 agent is an anti-human CD127 antagonist antibody or an antigen-binding fragment thereof which comprises the heavy chain variable domain comprising or consisting of the amino acid sequence set forth in SEQ ID No. 22, and the light chain variable domain comprising or consisting of the amino acid sequence set forth in SEQ ID No. 26.

In a particular aspect of the invention, the anti-CD127 agent is an anti-human CD127 antagonist antibody or an antigen-binding fragment thereof which comprises:

a heavy chain comprising or consisting of the amino acid sequence set forth in SEQ ID No. 20 or SEQ ID No. 27, and a light chain comprising or consisting of the amino acid sequence set forth in SEQ ID No. 21, SEQ ID No. 28 or SEQ ID No. 29,

said anti-CD127 antibody or an antigen-binding fragment thereof exhibiting ADCP activity against CD127-positive cells, in particular CD127-positive tumor cells, and has no ADCC activity. In an embodiment, the anti-human CD127 antibody or an antigen-binding fragment enhances the phagocytosis of CD127-positive cells, in particular CD127-positive tumor cells, by macrophages when administered to a patient, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

In a particular aspect of the invention, the anti-CD127 agent is an anti-human CD127 antagonist antibody or an antigen-binding fragment thereof which comprises:

a heavy chain comprising or consisting in the amino acid sequence set forth in SEQ ID No. 27 and a light chain comprising or consisting of the amino acid sequence set forth in SEQ ID No. 28 or SEQ ID No. 29, in particular a heavy chain comprising or consisting in the amino acid sequence set forth in SEQ ID No. 27 and a light chain comprising or consisting of the amino acid sequence set forth in SEQ ID No. 28,

said anti-CD127 antibody or an antigen-binding fragment thereof exhibiting ADCP activity against CD127-positive cells, in particular CD127-positive tumor cells, and has no ADCC activity. In an embodiment, the anti-human CD127 antibody or an antigen-binding fragment enhances the phagocytosis of CD127-positive cells, in particular CD127-positive tumor cells, by macrophages when administered to a patient, and is used for treating a patient having a cancer. Said anti-human CD127 antibody or an antigen-binding fragment is preferably an antagonist of the binding between IL-7 and CD127 as defined here above. In particular, said antibody or antigen-binding fragment thereof comprises a constant chain belonging to the subclass of IgG1, IgG2, IgG3 or IgG4, in particular the subclass of IgG4.

Table 1 details different combinations of amino acid sequences corresponding to several anti-CD127 agent used in the examples of the invention. It should be noted that this table is merely for illustrative purpose, and anti-CD127 agents disclosed herein should not be considered as the sole anti-CD127 agent that can be used according to the invention, or in a method according to the invention.

TABLE 1
Sequences SEQ ID No. of the CDRs, heavy and light chain
variable domains, and full heavy and light chains of several
antibodies used in the examples of the invention.

| Antibody | N13B2 | N13B2hVL6 | N13B2h3 |
|---|---|---|---|
| CDRs SEQ ID No. | SEQ ID No. | SEQ ID No. | SEQ ID No. |
| (HCDR1/HCDR2/ | 3/4/5/7/9/11 | 3/4/6/8/10/11 | 3/4/6/8/10/11 |
| HCDR3/LCDR1/ | | | |
| LCDR2/LCDR3) | | | |
| Heavy chain | SEQ ID No. 12 | SEQ ID No. 22 | SEQ ID No. 20 |
| variable domain | | | |
| SEQ ID No. | | | |
| Light chain | SEQ ID No. 16 | SEQ ID No. 26 | SEQ ID No. 21 |
| variable domain | | | |
| SEQ ID No. | | | |
| Full heavy chain | SEQ ID No. 12 + | SEQ ID No. 27 | SEQ ID No. 20 + |
| SEQ ID No. | SEQ ID No. 30 | | SEQ ID No. 30 |
| Full light chain | SEQ ID No. 16 + | SEQ ID No. 28 | SEQ ID No. 21 + |
| SEQ ID No. | SEQ ID No. 31 | | SEQ ID No. 31 |
| Antibody | N13B2hVL3 | N13B2hVL4 | N13B2hVL5 |
| CDRs SEQ ID No. | SEQ ID No. | SEQ ID No. | SEQ ID No. |
| (HCDR1/HCDR2/ | 3/4/6/8/10/11 | 3/4/6/8/10/11 | 3/4/6/8/10/11 |
| HCDR3/LCDR1/ | | | |
| LCDR2/LCDR3) | | | |
| Heavy chain | SEQ ID No. 22 | SEQ ID No. 22 | SEQ ID No. 22 |
| variable domain | | | |
| SEQ ID No. | | | |
| Light chain | SEQ ID No. 23 | SEQ ID No. 24 | SEQ ID No. 25 |
| variable domain | | | |
| SEQ ID No. | | | |
| Full heavy chain | SEQ ID No. 22 + | SEQ ID No. 22 + | SEQ ID No. 22 + |
| SEQ ID No. | SEQ ID No. 30 | SEQ ID No. 30 | SEQ ID No. 30 |
| Full light chain | SEQ ID No. 23 + | SEQ ID No. 24 + | SEQ ID No. 25 + |
| SEQ ID No. | SEQ ID No. 31 | SEQ ID No. 31 | SEQ ID No. 31 |

In a particular embodiment of the invention, the anti-CD127 agent to be used according to the invention or for use in a method according to the invention is provided as an isolated nucleic acid molecule or group of isolated nucleic acid molecules encoding an anti-CD127 agent as defined herein according to the invention, in particular an antibody or an antigen-binding fragment thereof. Particularly, said nucleic acid molecule or group of nucleic acid molecule encodes the light chain variable domain or the light chain of an antibody provided herein, and the heavy chain variable domain or heavy chain of an antibody provided herein, according to any of the definitions provided herein. In particular, the isolated nucleic acid molecules or the group of isolated nucleic acid molecules encodes:
a heavy chain variable domain comprising or consisting of the amino acid sequence set forth in SEQ ID No. 12, SEQ ID No. 13, SEQ ID No. 14, SEQ ID No. 15 or SEQ ID No. 22, in particular SEQ ID No. 15 or SEQ ID No. 22; and
a light chain variable domain comprising or consisting of the amino acid sequence set forth in SEQ ID No. 16, SEQ ID No. 17, SEQ ID No. 18, SEQ ID No. 19, SEQ ID No. 23, SEQ ID No. 24, SEQ ID No. 25 or SEQ ID No. 26, in particular SEQ ID No. 19 or SEQ ID No. 26.

More particularly, the isolated nucleic acid molecules or the group of isolated nucleic acid molecules encodes a heavy chain comprising or consisting of the amino acid sequence set forth in SEQ ID No. 20 or SEQ ID No. 27, and a light chain comprising or consisting of the amino acid sequence set forth in SEQ ID No. 21, SEQ ID No. 28 or SEQ ID No. 29.

In a particular embodiment of the invention, the anti-CD127 agent to be used according to the invention or for use in a method according to the invention is an anti-CD127 antibody or antigen-binding fragment thereof or antigen-binding mimetic thereof which has at least one of the following properties, in particular at least two, preferably at least three, and more preferably all the following properties: a) is an antagonist of the IL-7R signaling pathway induced by IL-7 (the antagonist capability of the anti-CD127 agent may be assessed by measuring the phosphorylation of STAT5 in presence or absence of the agent, the agent being considered as an antagonist of the IL-7R signaling pathway induced by IL-7 when in its presence it lowers the phosphorylation of STAT5), b) inhibits or reduces the activation of the phosphatidylinositol 3-kinase and/or the ERK signaling pathway induced by IL-7, c) does not increase the maturation of dendritic cells induced by TSLP (dendritic cells maturation may be assessed by determining an increase in the expression of cell surface marker CD40 and/or CD80 in TSLP receptor-positive cells treated with TSLP and with said agent compared to cells treated with TSLP alone), d) does not induce the internalization of CD127 and/or inhibit the IL7-induced internalization of CD127 (CD127—internalization designates the decrease of cell surface expression of CD127 induced by the presence of IL7; the cell surface expression of CD127 in cells incubated in the presence of the anti-CD127 agent is not reduced, or is not significantly reduced, relative to cell surface expression in cells incubated in otherwise identical conditions, but in the absence of the antibody. In particular embodiments, when incubated at 37° C. for 30 to 45 minutes in the presence of 50 ng/mL of antibody, the level of CD127 cell surface expression is at least 80%, preferably at least 90% of its level in cells incubated in the absence of the antibody. This effect may be observed in the absence of IL-7); e) binds to T cells. Details about how to assess if an anti-CD127 agent has or has not one of these properties may be found in WO2015/189302 and WO2018/104483 and WO2020154293. Anti-CD127 agents corresponding to this definition are for example disclosed in WO2015/189302, WO2018/104483.

In a particular embodiment, a particular therapeutic dose of the anti-CD127 agent is administered to a patient in need thereof, the dose being adapted to limit Adverse Events (AEs) while maintaining the efficacy of the agent for treating the disease of the patient.
Diseases to be Treated

The patients to be treated according to a method of the invention have, develop or are likely to develop a CD127-positive cancer. A CD127-positive cancer is a cancer wherein tumor cells express the Cluster of Differentiation 127 (CD127). “CD127-positive tumor cell” designates a tumor cell expressing CD127 at their cell surface. In most cases, CD127-positive cell expresses CD127 in a complex forming the IL-7R (IL-7R-positive cells) and/or in a complex forming the TSLPR (TSLPR-positive cells). A cancer may be classified in the subset of CD127-positive cancer by flow cytometry evaluation using a primary mouse monoclonal antibody directed against CD127 (mouse monoclonal anti-human CD127 clone [A019D5]. cat. 351304, Biolegend) and an irrelevant monoclonal mouse antibody (for example mouse monoclonal immunoglobulin IgGl,k, isotype control, cat.555746, BD Pharmingen) and a kit for quantitative determination of cell surface antigens, such as BD Quantibrite™ Beads(cat. 340495 BD Pharmingen): a CD127-positive cancer is defined by a Specific Antibody-Binding Capacity (SABC) strictly superior to zero. A CD127-positive cancer overexpressing CD127 is considered when CD127 expression level as measured by flow cytometry or RNA sequencing is higher than on healthy T cells or normal bone marrow control cells. In a particular embodiment of the invention, the patient to be treated has Leukemia, particularly CD127-positive Leukemia. Leukemia (also written “leukaemia”) is a malignant progressive disease in which the bone marrow and other blood-forming organs produce increased numbers of immature or abnormal leucocytes. These suppress the production of normal blood cells, leading to anemia and other symptoms. Leukemia are also referenced as “liquid cancers” or “blood cancers”. In the present description, the three terms “leukemia”, “liquid cancer” and “blood cancer” share the same meaning, except when expressly stated otherwise.

In a particular embodiment of the invention, the patient to be treated has an acute lymphoblastic leukemia, particularly a CD127-positive ALL. Acute lymphoblastic leukemia (ALL) is a cancer of the lymphoid line of blood cells. The lymphoid cell line is a type of white blood cell also referenced as Lymphocytes. Lymphocytes include natural killer cells (which function in cell-mediated, cytotoxic innate immunity), T cells (for cell-mediated, cytotoxic adaptive immunity), and B cells (for humoral, antibody-driven adaptive immunity).

In a particular embodiment of the invention, the patient to be treated has T-cell leukemia or T-cell ALL, in particular T-cell ALL, more particularly a CD127-positive T-cell leukemia or T-cell ALL.

In a particular embodiment of the invention, the patient to be treated has B-cell leukemia or B-cell ALL, in particular B-cell ALL more particularly a CD127-positive B-cell leukemia or B-cell ALL.

T-cell ALL is a cancer more particularly related to the provision of immature or abnormal T cells. B-cell ALL is a cancer more particularly related to the provision of immature or abnormal B cells.

In a particular embodiment of the invention, the patient to be treated has a cancer, particularly a CD127-positive cancer (comprising CD127-positive tumor cells), in particular a Leukemia (e.g. ALL) or solid cancer—wherein the IL-7R signaling pathway is constitutively active, as compared to normal (e.g., healthy) cells.

In a particular embodiment of the invention, the patient to be treated has a cancer, particularly a CD127-positive cancer (comprising CD127-positive tumor cells), in particular has a Leukemia (e.g. ALL) or solid cancer—wherein the IL-7R signaling pathway is not functional (e.g., cannot be activated), as compared to normal (e.g., healthy) cells.

In a particular embodiment of the invention, the patient to be treated has a cancer, particularly a CD127-positive cancer (comprising CD127-positive tumor cells), in particular has a Leukemia (e.g. ALL) or solid cancer, wherein the IL-7R signaling pathway is dysfunctional (e.g., cannot be activated, enhanced, inhibited, or reduced), as compared to normal (e.g., healthy) cells.

In a particular embodiment, the patient to be treated has a CD127-positive solid tumor, more particularly a CD127-positive solid cancer.

A used herein, the term solid tumor refers to an abnormal mass of tissue that usually does not contain cysts or liquid areas. Examples of solid cancers comprising solid tumor tumors are sarcomas, carcinomas, mesotheliomas and lymphomas.

Particularly, the patient to be treated has a CD127-positive mesothelioma.

In a particular embodiment of the invention, the patient to be treated has a Bcl-2-positive cancer, in particular has a Bcl-2-positive ALL. A Bcl-2-positive cancer corresponds to a cancer wherein tumor cell(s) express the Bcl-2 protein.

In a particular embodiment of the invention, the anti-CD127 agent is used (administered to a patient) in combination with a conventional treatment of cancer.

As used herein, the term “standard or conventional treatment” refers to any treatment of cancer (drug, radiotherapy, etc) usually administered to a subject who suffers from cancer.

In a particular embodiment of the invention, the anti-CD127 agent is used (administered to a patient), in particular simultaneously, separately or sequentially with at least one second therapeutic agent selected from the group consisting of a chemotherapeutic agent, a targeted cancer therapy agent, an immunotherapeutic agent and radiotherapy agent.

The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoproteinenediyneantiobioticchromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarb azine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposi de (VP-16); ifosfamide; mitomycin C; mitoxantrone; vincristine; vinorelbine; navelbine; novantrone; teniposi de; daunomycin; aminopterin; xeloda; ibandronate; CPT-1 1 ; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U. S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide(SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

The term “immunotherapeutic agent,” as used herein, refers to a compound, composition or treatment that indirectly or directly enhances, stimulates or increases the body's immune response against cancer cells and/or that decreases the side effects of other anticancer therapies.

Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively, the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-C SF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Combination compositions and combination administration methods of the present invention may also involve “whole cell” and “adoptive” immunotherapy methods. For instance, such methods may comprise infusion or re-infusion of immune system cells (for instance tumor-infiltrating lymphocytes (TILs), such as CC2+ and/or CD8+ T cells (for instance T cells expanded with tumor-specific antigens and/or genetic enhancements), antibody-expressing B cells or other antibody-producing or—presenting cells, dendritic cells (e.g., dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887, July 2001), optionally admixed with adjuvants such as alum, may also be components in such methods and combination compositions.

Particularly said immunotherapeutic agent is selected from the group consisting of an anti-CD3 agent, in particular anti-CD3 antibody, an anti-PD1 agent (particularly an anti-PD1 antibody), in particular an antagonist of PD1, more particulary an antagonist anti-PD1 antibody, an anti-PDL1 agent (particularly an anti-PDL1 antibody), in particular an antagonist of PDL1, more particularly an antagonist anti-PDL1 antibody, an anti-CTLA4 agent (particularly an anti-CTLA4 antibody), in particular an antagonist of CTLA4, more particularly an antagonist anti-CTLA4 antibody, an agonist of CD137, in particular an agonist anti-CD137 antibody, an anti-CLEC-1 agent (particularly an anti-CLEC-1 antibody), in particular an antagonist of CLEC-1, more particularly an antagonist anti-CLEC-1 antibody, an anti-VEGF agent, in particular an anti-VEGF antibody, anti-CD19 agent, in particular an anti-CD19 antibody, and anti-CD47 agent (particularly an anti-CD47 antibody), in particular an antagonist of CD47, more particularly an anti-CD47 antagonist antibody, an anti-SIRPa agent (particularly an anti-SIRPa antibody), in particular an antagonist of anti-SIRPa, more particularly an anti-SIRPa antagonist antibody, an anti-CD28 agent (particularly an anti-CD28 antibody), in particular an antagonist of anti-CD28, more particularly an anti-CD28 antagonist antibody an anti-Bcl-2 agent (in particular venetoclax also referenced as ABT199 or GDC-0199), an inhibitor of the tyrosine/kinase pathway such as venetoclax.

Radiotherapy may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In a particular embodiment of the invention, the at least one second therapeutic agent is selected from the group consisting of a cytotoxic agent, a chemotherapeutic agent, an anti-angiogenic agent, a cell-cycle control/apoptosis regulating agent, a hormonal regulating agent and an anti-cancer immunogen agent, in particular an anti-cancer antibody, more particularly a tumor-targeting antibody.

In a particular embodiment of the invention, the anti-CD127 agent is administered to a patient who is or has been or will be treated by chemotherapy, targeted therapy, radiation therapy, bone marrow transplantation, and/or engineered immune cells. In a particular embodiment, the anti-CD127 agent is administered to a patient who is resistant to a treatment by chemotherapy, targeted therapy, radiation therapy, bone marrow transplantation, and or engineered immune cells. Chemotherapy corresponds to the use of drugs to kill cancer cells, and is typically used as an induction therapy for children and adults with solid cancers and leukemias including acute lymphocytic leukemia. Chemotherapy can be used alone or in combination with other treatments. Chemotherapy drugs can also be used in the consolidation and maintenance phases. Targeted therapy corresponds to the use of targeted drug treatments focused on specific abnormalities present within cancer cells. By blocking these abnormalities, targeted drug treatments can cause cancer cells to die. Targeted therapy can be used alone or in combination with other treatments. Targeted therapy can be used as a consolidation therapy or maintenance therapy. Radiation therapy uses high-powered beams, such as X-rays or protons, to kill cancer cells. A bone marrow transplant, also known as a stem cell transplant, may be used as consolidation therapy or for treating relapse if it occurs. This procedure allows someone with cancer (in particular leukemia) to reestablish healthy bone marrow by replacing cancerous (in particular leukemic)bone marrow with cancer-free marrow (in particular leukemia-free marrow) from a healthy person. Engineered immune cells corresponds to the use of chimeric antigen receptor (CAR)-T cells. T cells may be engineered with CAR molecule. CARs are localized within the membrane of T cells. A CAR is a chimeric molecule comprising as its extracellular part an antibody-derived antigen recognition domain (usually an ScFv fragment), and as its intracellular domain a TCR-derived activating domain which confers to the T cells the capability to be activated against a specific tumor antigen (Gomes-Silva et al., Biotech J. 2017).

In a particular embodiment of the invention, the anti-CD127 agent is administered to a patient who experiences an inadequate response to at least one of the following treatments: chemotherapy, targeted therapy, radiation therapy, bone marrow transplantation and/or engineered immune cells.

In a particular embodiment of the invention, the patient to be treated has also an inflammatory central nervous system (CNS) disorder or disease (Alsadeq et al. Blood 2018). In particular, the patient to be treated has a CNS disorder or disease and a cancer, in particular an ALL, more particularly a B-ALL, even more particularly an ALL as described below.

In a particular embodiment of the invention, the patient to be treated has a ALL selected from the following group: CD127+ ALL, CD127 overexpressing ALL (which can be determined by comparing the CD127 expression in ALL cells and in healthy bone marrow cells), CD127 and/or JAK-STAT signaling pathway mutated ALL (as compared to healthy cells) (a ALL wherein the IL-7R signaling pathway is dysfunctional (e.g. cannot be activated, enhanced, inhibited, or reduced), as compared to normal (e.g. healthy) cells), including BCR-ABL1—like ALL, as well as B cell precursor ALL bearing the following cytogenetics: t(1;19), t(12,21), MLL-rearrangements, Hyperdiploid karyotypes, trisomy 4 and trisomy 10. In a particular embodiment, the invention relates to anti-CD127 antibodies or antigen-binding fragments thereof, having (i.e. increasing as compared to a negative control) Antibody Dependent Cellular Phagocytosis (ADCP) activity macrophages targeting CD127-positive cancer cells, in particular wherein said activity is achieved by or involves macrophages, for the treatment of ALL selected from the group consisting of CD127 wild type T-ALL (HPB-ALL cell line), CD127-mutated T-ALL (DND41 cell line), t(1;19) B-ALL (697 cell line), t(12;21) B-ALL (REH cell line) and t(5;12) B-ALL (NALM6 cell line).
Combinations of Agents

In an embodiment of the invention, the anti-CD127 agent for use in the method of the invention or for use according to the invention is administered to the patient in combination with at least a second active ingredient, like another therapeutic agent. Said second active ingredient includes but is not limited to probiotics and therapeutic agents as described below. The anti-CD127 agent for use in the method of the invention or for use according to the invention may be administered with several, different, second active ingredients.

The present invention thus also relates to the combination of an anti-CD127 agent for use in the method of the invention or for use according to the invention, in combination with at least one second active ingredient. The combination is for use in the treatment of CD127-positive cancer by enhancing the phagocytosis of CD127-positive tumor cells. The second active ingredient does not necessarily have any effect on the phagocytosis of CD127-positive tumor cells, but may have other properties useful in the treatment of cancer.

In some embodiments, the anti-CD127 agent for use in the method of the invention or for use according to the invention is administered to the patient in combination with a standard (conventional) treatment. The present invention relates thus to the combination of an anti-CD127 agent for use in the method of the invention or for use according to the invention with a conventional treatment for use in the treatment of cancer. As used herein, the term “standard or conventional treatment” refers to any treatment of cancer (drug, radiotherapy, etc) usually administered to a patient who suffers from cancer.

In some embodiments, the anti-CD127 agent for use in the method of the invention or for use according to the invention is administered to the subject in combination with at least one further therapeutic agent, e.g., for treating cancers. Such administration may be simultaneous, separate or sequential. For simultaneous administration, the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, anti-angiogenic agents, anti-cancer immunogen agents, cell cycle control/apoptosis regulating agents, hormonal regulating agents, and other agents described below.

In some embodiments, the anti-CD127 agent for use in the method of the invention or for use according to the invention is used in combination with a chemotherapeutic agent, a targeted cancer therapy agent, an immunotherapeutic agent or radiotherapy agent.

In some embodiments, the anti-CD127 agent for use in the method of the invention or for use according to the invention is used in combination with a chemotherapeutic agent or targeted therapy agent. The present invention relates thus to the combination of an antagonist of CD127 with a chemotherapeutic agent or targeted therapy agent for use in the treatment of CD127-positive cancer.

The second therapeutic agent may be selected from the list consisting of anti-CD3 agent, in particular anti-CD3 antibody, an anti-PD1 agent, in particular antagonist anti-PD1 antibody, an anti-PDL1 agent, in particular an antagonist anti-PDL1 antibody, an anti-CTLA4 agent, in particular an antagonist anti-CTLA4 antibody, an agonist of CD137, in particular an agonist anti-CD137 antibody, an anti-VEGF agent, in particular an anti-VEGF antibody, an anti-CLEC-1 agent, in particular an anti-CLEC-1 antibody, an anti-CD28 agent, in particular an anti-CD28 antibody, an anti-CD19 agent, in particular an anti-CD19 antibody, and anti-CD47 agent, in particular an anti-CD47 antibody, more particularly an anti-CD47 antagonist agent, even more particularly an anti-CD47 antagonist antibody, an anti-SIRPa antagonist agent, more particularly an anti-SIRPa antagonist antibody, an anti-Bcl-2 agent (in particular venetoclax also referenced as ABT199 or GDC-0199), an inhibitor of the tyrosine/kinase pathway, Dexamethasone, rituximab, trastuzumab, cetuximab. Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Ti sagenlecleucel); Vincristine, Marqibo (Vincristine Sulfate Liposome); Mercaptopurine (or Purinethol or Purixan); Methotrexate Sodium (or Trexall); Nelarabine; Oncaspar (or Pegaspargase/PEG-Asparaginase); Ponatinib Hydrochloride; Prednisone; Purinethol (Mercaptopurine); Vincristine Sulfate or Vincristine Sulfate Liposome. In a particular embodiment of the invention, the second therapeutic agent is selected from the list consisting of Dexamethasone, an anti-CD47 antagonist antibody, an inhibitor of the tyrosine kinase pathway (Malard and Mohty, The Lancet 2020).

As used herein, an anti-CD47 antagonist agent, in particular an anti-CD47 antagonist antibody, has its general meaning in the art and refers to any compound, natural or synthetic, that blocks, suppresses, or reduces the biological activity of CD47. In particular, the CD47 antagonist inhibits the interactions between CD47 and one of its ligand, in particular SIRPa.

In a particular embodiment, it is provided a combination of an anti-CD127 agent for use in the method of the invention or for use according to the invention, in combination with at least one second active ingredient, the at least one second active ingredient being an anti-CD47 antagonist agent, in particular an anti-CD47 antagonist antibody or antigen-binding fragment thereof.

In a particular embodiment, it is provided a combination of an anti-CD127 agent for use in the method of the invention or for use according to the invention, in combination with at least a second active ingredient, the at least one second active ingredient being an anti-SIRPa antagonist agent, in particular an anti-SIRPa antagonist antibody, or antigen-binding fragment thereof

In a particular embodiment, it is provided a combination of an anti-CD127 agent for use in the method of the invention or for use according to the invention, in combination with at least one second active ingredient, the at least one second active ingredient being an anti-Bcl-2 agent, in particular Venetoclax (see for example Richard-Carpentier et al., Clin Lymphoma Myeloma Leuk. 2020). In a particular embodiment, this combination of compounds (i.e., an anti-CD127 agent and an anti-Bcl-2 agent) is administered in combination with chemotherapy, in particular simultaneously, separately or sequentially.

An inhibitor of the tyrosine/kinase pathway is a pharmaceutical drug that inhibits tyrosine kinases. These inhibitors are usually also called tyrphostins. These inhibitors usually either compete with adenosine triphosphate (ATP), the phosphorylating entity of the tyrosine kinase, the substrate of the tyrosine kinase, or modify the confirmation of the tyrosine kinase, thereby modifying its phosphorylating activity.

In a particular embodiment, it is provided a combination of an anti-CD127 agent for use in the method of the invention or for use according to the invention, in combination with at least a second active ingredient, the at least one second active ingredient being an inhibitor of the tyrosine/kinase pathway, like but not limited to the pharmaceutical drugs referenced under the acronym TKI (tyrosine kinase inhibitor). Examples of TKI comprises but are not limited to tyrphostins, imatinib, gefitinib, erlotinib, dasatinib, sunitinib, adavosertib, lapatinib.

In a particular embodiment, it is provided a combination of an anti-CD127 agent for use in the method of the invention or for use according to the invention, in combination with at least a second active ingredient, the at least one second active ingredient being an anti-Bcl-2 agent. An anti-Bcl-2 agent is an inhibitor of the anti-apoptotic protein B-cell lymphoma 2 (Bcl-2), with potential pro-apoptotic and antineoplastic activities. In particular embodiment of the invention, the anti-Bcl-2 agent is selected from the group consisting of venetoclax (also referenced as ABT199 or GDC-0199), oblimersen, navitoclax, obatoclax, obatoclax mesylate. Anti-Bcl-2 agents are known to induce tumor regression by blocking the cellular pathway induced by Bcl-2 which inhibits cellular apoptosis. Targeting this protein has been demonstrated to have high efficacy in cancer therapy, in particular in ALL therapy.

In a particular embodiment, it is provided a combination of an anti-CD127 agent for use in the method of the invention or for use according to the invention, in combination with at least a standard of care treatment (SOC treatment). SOC Treatment may be considered as the treatment that is accepted by medical experts as a proper treatment for a certain type of disease and that is widely used by healthcare professionals. Also called best practice, standard medical care, and standard therapy. In a particular embodiment, it is provided a combination of an anti-CD127 agent for use in the method of the invention or for use according to the invention, in combination with at least a standard of care treatment of a specific cancer type, in particular of Acute Lymphoblastic Leukemia (ALL), more particularly T-cell ALL or B-cell ALL. Standard of care treatment included the administration to a patient in need thereof of chemotherapeutic agents such as pemetrexed (Alimta), cisplatin, carboplatin, gemcitabine (Gemzar), vinorelbine, doxorubicin, paclitaxel; or radiation therapy, or immunotherapeutic agents such as avastin (bevacizumab), pembrolizumab (keytruda), nivolumab (opdivo), ipilimumab (yervoy) In a particular embodiment, it is provided a combination of an anti-CD127 agent for use in the method of the invention or for use according to the invention, in combination with at least a standard of care treatment (SOC treatment) of ALL comprising at least Dexamethasone, Oncaspar (known under references Pegaspargase/PEG-Asparaginase) and Vincristine. In a more particular embodiment, it is provided a combination of an anti-CD127 agent for use in the method of the invention or for use according to the invention, in combination with Dexamethasone, Oncaspar (known under references Pegaspargase/PEG-Asparaginase) and Vincristine.

In a particular embodiment, it is provided a combination of an anti-CD127 agent for use in the method of the invention or for use according to the invention, in combination with at least a standard of care treatment (SOC treatment) of mesothelioma comprising:
A chemotherapy agent such as folic acid analog including Pemetrexed (Alimta), Cisplatin, Carboplatin, Gemcitabine (Gemzar), Vinorelbine, Doxorubicin, Paclitaxel
An immunotherapeutic agent such as an anti-VEGF agent including Avastin (Bevacizumab), an anti-PD-1 agent including Pembrolizumab (Keytruda), nivolumab (Opdivo), an anti-CTLA4 agent including Ipilimumab (Yervoy).

In a particular embodiment of the invention, it is provided a combination of compounds comprising:
a) an anti-CD127 agent, in particular an anti-CD127 antibody or antigen-binding fragment thereof or antigen-binding antibody mimetic, that has Antibody Dependent Cellular Phagocytosis (ADCP) activity on CD127-positive tumor cells, in particular by macrophages, and has no ADCC activity; and
b) dexamethasone and/or oncaspar and/or vincristine, in particular dexamethasone and oncaspar and vincristine, and/or an anti-CD47 antagonist antibody, and/or an anti-SIRPa antibody and/or an anti-BCL2 agent and/or an inhibitor of the tyrosine/kinase pathway, in particular dexamethasone.

In a particular embodiment of the invention, it is provided a combination of compounds comprising:
a) an anti-human CD127 antagonist antibody or an antigen-binding fragment thereof which comprises:

a VH chain comprising at least the following amino acid sequences:
VHCDR1 SEQ ID No. 3;
VHCDR2 SEQ ID No. 4;
VHCDR3 SEQ ID No. 5 or SEQ ID No. 6;

and a VL chain comprising at least the following amino acid sequences:
VLCDR1 SEQ ID No. 7 or SEQ ID No. 8;
VLCDR2 SEQ ID No. 9 or SEQ ID No. 10;
VLCDR3 SEQ ID No. 11,
   said anti-CD127 antibody or an antigen-binding fragment thereof exhibiting ADCP activity against CD127-positive cells, in particular CD127-positive tumor cells, in particular by macrophages , and has no ADCC activity; and
b) dexamethasone and/or oncaspar and/or vincristine, in particular dexamethasone and oncaspar and vincristine and/or an anti-CD47 antagonist antibody, and/or an anti-SIRPa antibody and/or an anti-BCL2 agent, and/or an inhibitor of the tyrosine/kinase pathway, in particular dexamethasone.

In a particular embodiment, the anti-human CD127 antagonist antibody present in the combination of compounds comprises:

a VH chain comprising at least the following amino acid sequences:
VHCDR1 SEQ ID No. 3;
VHCDR2 SEQ ID No. 4;
VHCDR3 SEQ ID No. 5;
   and a VL chain comprising at least the following amino acid sequences:
VLCDR1 SEQ ID No. 7;
VLCDR2 SEQ ID No. 9;
VLCDR3 SEQ ID No. 11.

In a particular embodiment, the anti-human CD127 antagonist antibody present in the combination of compounds comprises:

a VH chain comprising at least the following amino acid sequences:
VHCDR1 SEQ ID No. 3;
VHCDR2 SEQ ID No. 4;
VHCDR3 SEQ ID No. 6;
   and a VL chain comprising at least the following amino acid sequences:
VLCDR1 SEQ ID No. 8;
VLCDR2 SEQ ID No. 10;
VLCDR3 SEQ ID No. 11.

In a particular aspect of the invention, the combination of compounds comprises:

i) an anti-CD127 agent which is an anti-human CD127 antagonist antibody or an antigen-binding fragment thereof which comprises:

a heavy chain variable domain comprising or consisting of the amino acid sequence set forth in SEQ ID No. 12, SEQ ID No. 13, SEQ ID No. 14, SEQ ID No. 15 or SEQ ID No. 22, in particular SEQ ID No. 15 or SEQ ID No. 22; and

a light chain variable domain comprising or consisting of the amino acid sequence set forth in SEQ ID No. 16, SEQ ID No. 17, SEQ ID No. 18, SEQ ID No. 19, SEQ ID No. 23, SEQ ID No. 24, SEQ ID No. 25 or SEQ ID No. 26, in particular SEQ ID No. 19 or SEQ ID No. 26;

and

ii) dexamethasone and/or oncaspar and/or vincristine, in particular dexamethasone and oncaspar and vincristine and/or an anti-CD47 antagonist antibody, and/or an anti-SIRPa antibody and/or an anti-BCL2 agent, and/or an inhibitor of the tyrosine/kinase pathway, in particular dexamethasone.

In a particular embodiment, the anti-human CD127 antagonist antibody present in the combination of compounds comprises:

the heavy chain variable domain comprising or consisting of the amino acid sequence set forth in SEQ ID No. 12, and the light chain variable domain comprising or consisting of the amino acid sequence set forth in SEQ ID No. 16.; or

the heavy chain variable domain comprising or consisting of the amino acid sequence set forth in SEQ ID No. 15, and the light chain variable domain comprising or consisting of the amino acid sequence set forth in SEQ ID No. 19; or

the heavy chain variable domain comprising or consisting of the amino acid sequence set forth in SEQ ID No. 22, and the light chain variable domain comprising or consisting of the amino acid sequence set forth in SEQ ID No. 26.

In a particular aspect, the invention, the combination of compounds comprises:

an anti-CD127 agent which is an anti-human CD127 antagonist antibody or an antigen-binding fragment thereof which comprises:

a heavy chain comprising or consisting of the amino acid sequence set forth in SEQ ID No. 20 or SEQ ID No. 27 and a light chain comprising or consisting of the amino acid sequence set forth in SEQ ID No. 21, SEQ ID No. 28 or SEQ ID No. 29;

and

dexamethasone and/or oncaspar and/or vincristine, in particular dexamethasone and oncaspar and vincristine and/or an anti-CD47 antagonist antibody, and/or an anti-SIRPa antibody and/or an anti-BCL2 agent, and/or an inhibitor of the tyrosine/kinase pathway, in particular dexamethasone.

In a particular embodiment, the anti-human CD127 antagonist antibody present in the combination of compounds comprises or consists a heavy chain comprising or consisting in the amino acid sequence set forth in SEQ ID No. 27 and a light chain comprising or consisting of the amino acid sequence set forth in SEQ ID No. 28 or SEQ ID No. 29, in particular a heavy chain comprising or consisting in the amino acid sequence set forth in SEQ ID No. 27 and a light chain comprising or consisting of the amino acid sequence set forth in SEQ ID No. 28.

Particular Methods for Treating CD127-Positive Cancers

In some embodiments, the method of the invention or the use of an anti-CD127 agent as described herein is for treating a patient having a CD127-positive cancer and who has deleterious side effects associated to the treatment of its cancer by a conventional treatment, such as chemotherapy and/or administration of an anti-CD3, anti-CD19 and/or andi-CD47 compound. Deleterious side effect may include cytokine release syndrome, severe neurotoxicity, sinusoidal obstruction syndrome, hepatic toxicity, lymphodepletion.

In a particular embodiment, the method of the invention or the use of the anti-CD127 agent as described herein is for treating a patient having a cancer as a complementary treatment, in particular ALL, in particular a CD127-positive cancer, the patient being or having been treated with a first treatment, in particular chemotherapy, chemotherapy with stem cell transplant, radiation therapy, surgery and/or immunotherapy.

In a particular embodiment, the method of the invention or the use of the anti-CD127 agent as described herein is for treating a patient having a cancer as a complementary treatment, in particular a CD127-positive cancer, in particular ALL, the method of the use comprising a first step of determining if the patient has a CD127-positive cancer, in particular if the patient has a CD127-positive Leukemia or solid cancer, particularly a a CD127-positive ALL.

In a particular embodiment the anti-CD127 agent of the invention is administered to said patient after the patient has been evaluated as having CD127-positive tumor cells.

Particularly, a biological sample, in particular a blood sample, previously obtained from the patient is assayed for the presence of CD127 positive cells.

In a particular embodiment, the method for treating a patient having a CD127-positive cancer, according to the invention comprises the steps of:
a) Determining if the patient has CD127-positive tumor cells,
b) When the patient has CD127-positive tumor cells, administrating to the patient an effective amount of an anti-CD127 agent, in particular an anti-CD127 antibody or antigen-binding fragment thereof or antigen-binding antibody mimetic, that has Antibody Dependent Cellular Phagocytosis (ADCP) activity against CD127-positive tumor cells, in particular by macrophages, and that does not have Antibody Dependent Cytotoxic Activity (ADCC), in particular against immune cells, more particularly against T cells.

In a particular embodiment, the method of the invention or the use of the anti-CD127 agent as described herein is for treating a patient having an ALL and having a lymphodepletion due to a treatment with a conventional treatment of ALL.

In a particular embodiment, the method of the invention or the use of the anti-CD127 agent as described herein is for treating a patient having an ALL, and before metastatic cells issued from the ALL pass within the central nervous system. The use of the anti-CD127 agent may indeed prevent metastatic cells to spread to the central nervous system, on the contrary to conventional treatment of ALL.

In a particular embodiment, the method of the invention or the use of the anti-CD127 agent as described herein is for treating a patient having an ALL with metastatic cells, said metastatic cells having not reached the central nervous system. The use of the anti-CD127 agent may indeed prevent metastatic cells to spread to the central nervous system, on the contrary to conventional treatment of ALL

In a particular embodiment, the method of the invention or the use of the anti-CD127 agent as described herein is for treating a patient having an ALL and who cannot be treated with a conventional treatment of ALL, in particular due to the toxicity of the conventional treatment or due to the non-responding status of the patient to such treatment or to acquired resistance to such treatment.

In a particular embodiment, the method of the invention or the use of the anti-CD127 agent as described herein is for treating a patient having an ALL, and who does not positively respond to a treatment with a conventional treatment of ALL, in particular patient who does not respond to at least one of the following drug: an anti-CD3 agent, in particular anti-CD3 antibody, an anti-CD19 agent, in particular an anti-CD19 antibody, and an anti-CD47 agent, in particular an anti-CD47 antibody, in particular an anti-CD47 antagonist agent, in particular an anti-CD47 antagonist antibody, an anti-SIRPa antagonist agent, more particularly an anti-SIRPa antagonist antibody, an anti-Bcl-2 agent, in particular venetoclax, an inhibitor of the tyrosine/kinase pathway, Dexamethasone, rituximab, trastuzumab, cetuximab Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Dexamethasone; Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Vincristine, Marqibo (Vincristine Sulfate Liposome); Mercaptopurine (or Purinethol or Purixan); Methotrexate Sodium (or Trexall); Nelarabine; Oncaspar (or Pégaspargase/PEG-Asparaginase); Ponatinib Hydrochloride; Prednisone; Purinethol (Mercaptopurine); Vincristine Sulfate or Vincristine Sulfate Liposome. In a particular embodiment, the patient does not positively respond to a treatment with a compound selected from the list consisting of an anti-CD47 antagonist antibody, an anti-SIRPa antibody, an anti-Bcl-2 agent, oncaspar, vincristine, an inhibitor of the tyrosine/kinase pathway and Dexamethasone, in particular Dexamethasone, more particularly dexamethasone and oncaspar and vincristine.

In some embodiments, the method of the invention or the use of an anti-CD127 agent as described herein is for treating a patient having a CD127-positive cancer and who has not been treated yet with a conventional treatment known for having deleterious side effects, such as chemotherapy and/or administration of an anti-CD3 compound and/or an anti-CD19 compound and/or administration of an anti-CD47 compound, and/or administration of an anti-SIRPa compound and/or administration of an anti-Bcl-2 agent and/or administration of dexamethasone, and/or an inhibitor of the tyrosine/kinase pathway.

In some embodiments, the method of the invention or the use of an anti-CD127 agent as described herein is for treating a patient having a CD127-positive cancer and who is resistant to a conventional treatment, such as administration of dexamethasone.

In some embodiments, the method of the invention or the use of an anti-CD127 agent as described herein is for treating a patient having a CD127-positive cancer which has not metastased yet.

In some embodiments, the method of the invention or the use of an anti-CD127 agent as described herein is for treating a patient having a CD127-positive cancer, the patient being a child (i.e. being less than 15-year old).

In some embodiments, the method of the invention or the use of an anti-CD127 agent as described herein is for treating a patient having a CD127-positive cancer, wherein the IL-7R signaling pathway is dysfunctional, in particular not functional or constitutively activated.

Method for Selecting a Patient to be Treated with an Anti-CD127 Agent in Replacement of a Treatment with an Anti-CD19 Agent, like Blinatumomab, or Dexamethasone

In an embodiment of the invention, it is provided a method to select a patient having a CD127-positive cancer, in particular an ALL, more particularly T cell ALL or B cell ALL, and most particularly CD127 overexpressing Acute Lymphoblastic Leukemia (ALL), CD127 and/or JAK-STAT pathway mutated ALL, BCR-ABL1—like ALL, and B cell precursor ALL bearing one the following cytogenetics: t(1;19), t(12,21), MLL-rearrangements, hyperdiploid karyotypes, trisomy 4 and trisomyl0, and who can be treated by administration of an anti-CD127 agent as defined in the present description, the method comprising the measurement of the expression of CD127 by tumor cells, and the measurement of the expression of CD19 by tumor cells, a patient being able to be treated by the administration of an anti-CD127 agent when i) the tumor cells express CD127 and ii) the tumor cells do not express CD19, meaning that the ALL is resistant to a treatment by an anti-CD19 agent, like Blinatumomab.

In an embodiment of the invention, it is provided a method to select a patient having a CD127-positive cancer, in particular an ALL, more particularly T cell ALL or B cell ALL, and most particularly CD127 overexpressing Acute Lymphoblastic Leukemia (ALL), CD127 and/or JAK-STAT pathway mutated ALL, BCR-ABL1—like ALL, and B cell precursor ALL bearing one the following cytogenetics: t(1;19), t(12,21), MLL-rearrangements, hyperdiploid karyotypes, trisomy 4 and trisomyl0, and who can be treated by administration of an anti-CD127 agent as defined in the present description, the method comprising the measurement of resistance of the tumor cells to a treatment with dexamethasone, and when the tumor cells are resistant to dexamethasone, the method further comprise the measurement of the resistance of the tumor cells to a treatment with an anti-CD127 agent as described herein, alone or in combination with dexamethasone.

Method for Determining the Likelihood of a Patient to Respond to a Treatment with an Anti-CD127 Aagent

In an embodiment the invention relates to a method of determining the likelihood of a patient diagnosed with cancer to benefit from a treatment with an anti-CD127 agent, wherein a biological sample, in particular a blood sample, previously obtained from the patient is assayed for the presence of CD127 positive cells and wherein in case such cells are contained in the sample the patient's condition is considered likely to benefit from a treatment with an anti-CD127 agent as described herein.

In an embodiment the invention relates to a method of determining the likelihood of a patient diagnosed with cancer to benefit from a treatment with an anti-CD127 agent, wherein a biological sample, in particular a blood sample, previously obtained from the patient is assayed for the presence of CD127 positive cells and wherein in case such cells are contained in the sample, the activity of the IL-7R signaling pathway is measured and compared with normal cells to assess if this pathway is dysfunctional, in particular not functional or constitutively active, and when the pathway is dysfunctional, the patient's condition is considered likely to benefit from a treatment with an anti-CD127 agent as described herein.

### EXAMPLES

Material and Methods

ALL patient samples, human leukemic cell lines. Leukemia patients were treated according to ALL-Berlin-Frankfurt-MUnster (BFM) 2000 or 2009 protocols after informed consent in accordance with the Declaration of Helsinki. The study was approved by the ethical committee of the Christian-Albrechts-University Kiel (D437/17). Jurkat, THP1, HPB-ALL, MSTO-211H and DND41 T-ALL cell lines were purchased from ATCC. 697, NALM6 and REH B-ALL cell lines were purchased from DSMZ (Leibniz Institute, Germany). All cells were tested and found free from mycoplasma.

Mice. NOD.Cg-Prkdcscid Il2rgtm1Wjl/SzJ mice expressing Hc (NSG-Hc) mice were generated by backcrossing the intact Hc gene from the NOD-CBALs-Hcl/Lt congenic strain into the NSG strain, in collaboration with Lenny Shultz (Jackson Laboratories, Bar Harbor, USA). NSG-Hc mice were bred under pathogen-free conditions at Schleswig-Holstein Kiel University and xenografts were generated in accordance with governmental regulations (Schleswig-Holstein Ministerium fur Energiewende, Landwirtschaft, Umwelt, Natur and Digitalisierung): leukemic cells were injected intravenously into female NSG-Hc mice (6-10 weeks of age) and leukemic engraftment was followed by detection of human CD45+/murine CD45−/human CD19+ cells in the peripheral blood via flow cytometry analysis. Animals were sacrificed when showing signs of overt leukemia (detection of >75% leukemic blasts in the peripheral blood or clinical signs of leukemia including loss of weight or activity, organomegaly, hindlimb paralysis). Mouse survival was assessed using Kaplan-Meyer log-rank statistics.

In minimal residual disease (MRD) experiments, mice were injected with 10,000 BCP-ALL patient derived xenograft cells (n=10) of E2A-PBX1 positive patients (n=2 patients) and antibody N13B2-HVL6 (5mg/kg), EFFI-3-VH3VL3 (1mg/kg) or vehicle were injected intravenously every 3 days starting from day 1 until day 21, when injections were applied every 14 days. Minimal residual disease was measured by PCR for patient-specific immunoglobulin/B-cell receptor rearrangements in bone marrow samples isolated from PDX mice.

In overt leukemia experiments, mice were injected with 1 million BCP-ALL patient derived xenograft cells (n=10) of E2A-PBX1 positive patients (n=2 patients). Once the leukemic engraftment (determined by detection of hCD45+/hCD19+/mCD45− cells in the peripheral blood) was superior to 1%, antibody N13B2-hVL6 (5mg/kg), EFFI-3-VH3VL3 (1 mg/kg) or vehicle were injected intravenously every 3 days seven times, and subsequently every 14 days.

Antibodies used in treatments. All antibodies were generated at OSE and found free from endotoxin.

Phagocytosis assays. In vitro phagocytosis assays were performed by 1-hour coculture of 2,5×10⁴ human primary M1 or THP1 macrophages labeled with CellTrackerGreen (ThermoFisher, Waltham, Mass., USA, 1/2000, 20 min at 37° C.) and 5×10⁴ leukemic cells labeled with CPD (ThermoFisher, 1/2000 10 min at 37° C.) in serum-free RPMI. Phagocytosis was analyzed by a CytoFLEX flow cytometer (Beckman, Brea, Calif., USA) and analysis using FlowJo software (TreeStar, BD Life Sciences Franklin Lakes, N.J., USAs). The phagocytic index was calculated as follows: fold change of percentage of CPD+ cells in CTG+ macrophages compared to the one detected by treatment with istotype control multiplied by the fold change in geometric mean in APC fluorescence (CPD) in CTG+ macrophages compared to the one detected with isotype control. The normalized phagocytic index defines the maximal response by each independent donor against each cell line as 100%, as described in Ring et al. PNAS 2017.

Visualization of engulfed leukemic cells (CPD+) by M1 human macrophages (CTG+) was investigated in parallel to flow cytometry analysis using a Nikon ECLIPSE Ti2 microscope using the NIS-Elements software (Nikon, Minato City, Tokyo, Japan).

Time-lapse microscopy experiments were performed in Ibidi 18-well plates coated with Poly-L-Lysine 0,001%. M1 human macrophages were labeled with pHrodo-SE (ThermoFisher) diluted at 1/333000 for 30 min at 37° C. and seeded at 0,1×106 cells per well. Images were taken every 5 minutes for 4 hours and every 15 minutes for 10 hours by a Nikon ECLIPSE Ti2 microscope using the NIS-Elements software (Nikon).

Phase I study. A First in Human, Phase 1, randomized, double blind, placebo-controlled, single center study (EUDRACT number 2018-001832-22) was conducted in 63 healthy adult male and female volunteers in order to evaluate the safety, tolerability, PK, pharmacodynamics and immunogenicity of single and repeat ascending doses of N13B2-hVL6. N13B2-hVL6 was either administered at single dose (0.002, 0.02, 0.2, 1, 4, or 10 mg/kg IV) or two doses were given 2 weeks apart (6 or 10 mg/kg) and blood samples were drawn in order to evaluate lymphocyte counts after treatment at each time point of the study.

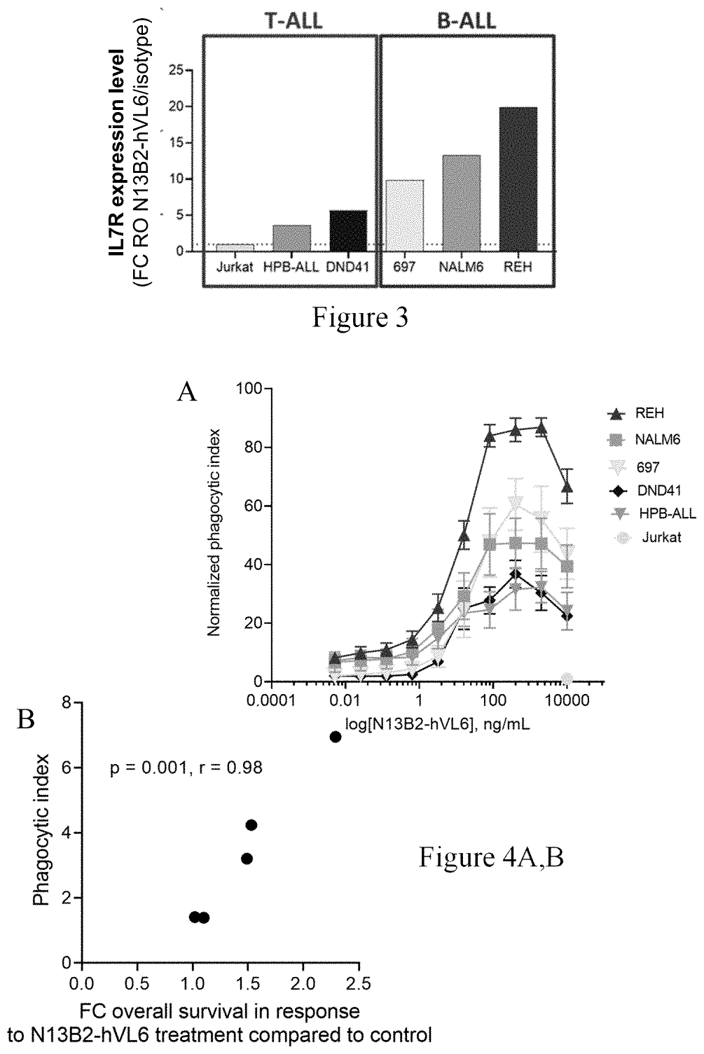

Quantification of specific antibody binding to CD127. N13B2-hVL6 and a corresponding isotype control (MOTA IgG4 S228P) were used to label cells (10 ug/mL each, 30 min at 4° C.). A secondary anti-hurnan IgG Fc [HP6017] Mouse IgG2a, κ PE antibody (BioLegend, San Diego, Calif., USA, cat# 409304) was used to detect the level of binding of N13B2-hVL6 to the different cell lines. The fold change of receptor occupancy of N13B2-nVL6 (FC RO) in FIG. 3 was calculated as the fold change of Geometric Mean PE fluorescence of N13B2-nVL6 labelled cells compared to that of isotype control labelled cells.

ADCC assay. 1 million human freshly isolated T cells were labelled with 15 uL ⁵¹Cr (5 mCi/ml, PerkinElmer, Waltham, Mass., USA, cat# NEZ030001MC) for 1 h at 37° C., 5% CO2. T cells were then washed until radioactivity (measured by radioactive gamma counter) was absent in the supernatant. 25 μl/well of T cells-⁵¹Cr target cells at 0.4 million cells/mL (10,000 cells/w) were seeded on P96-microtiter plate (flat bottom). 25 μl/well of anti-hCD127 antibodies were added in triplicate at 200 ng/mL (100 ng/mL final concentration) and left to incubate for 15 min at RT. Eventually, 50 μl of NK cells at 2 million cells/mL (100,000 cells/w) (ratio 10 NK cells:1 T cell) were added and incubated for 4 hours at 37° C., 5% CO2. As positive control of cytotoxicity, 75 μL of SDS 10% was added in 3 wells for 10 min before reading. Eventually, 25 μL/w supernatant were placed into radioactive reader plate and 100 μL/w Microscint Scintillant (PerkinElmer cat# 60136211) were added. Release of ⁵¹Cr in supernatant was measured by radioactive gamma counter in counts per minutes (cpm). Specific ADCC in FIG. 10B corresponds to sample well mean cpm (triplicates).

For ADCC assay illustrated on FIG. 10C, effector cells, NK cells, were purified by negative selection (NK isolation kit, Miltenyi) from human PBMC and incubated with 150 UI/mL of IL-2 overnight at 37° C., 5% CO2. The day of reaction, target cells CD127-positive-Luc+ cells (CD127+ luciferase+ cells) were plated at 25000 cells/well in white flat-P96 plates (Greiner, ref 655098) and incubated with the antibodies for 15min at room temperature. Then, 250000 NK cells/well were added to the reaction plate and incubated for 4 h at 37° C., 5% CO2. After incubation, substrate One Glo Luciferase product (2×) was added to each well to measure the number of live CD127-positive-Luc+ cells by chemiluminescence in a TECAN plate reader. Results were expressed in specific toxicity (=percentage of cytotoxicity between the maximal cytotoxicity (CD127+-Luc+ with SDS10%) and the minimal cytotoxicity (CD127-positive-Luc+ cells with NK, without antibody)).

ELISA binding to FcγR. For binding ELISA assay, recombinant hCD64/FcγRI (R&Dsystems, Minneapolis, Minn., USA; reference 1257-FC-050) or hCD32a/FcγRIIa (R&Dsystems, Minneapolis, Minn., USA; reference 1330-CD-050) or hCD16a/FcγRIIIa (R&Dsystems, Minneapolis, Minn., USA; reference 4325-FC-050) was immobilized on plastic at 2 μg/ml in borate buffer (pH9) and purified antibody were added to measure binding. After incubation and washing, peroxidase-labeled donkey anti-human IgG (Jackson Immunoresearch; USA; reference 709-035-149) was added and revealed by conventional methods.

P-STAT5 probing by flow cytometry: Cells were starved overnight in TexMacs medium (Miltenyi, Bergisch Gladbach, Germany; reference 130-097-196). After 30 min incubation in TexMacs medium (37° C.) supplemented with anti-CD127 agents, cells were stimulated with 5 ng/mL rhIL-7 (AbDSerotec, Raleigh, North Carolina, USA; reference PHP046) at 37° C. The reaction was then stopped by incubation on ice for 5 min and cells were fixed and permeabilized using the Phospho-Flow protocol from BD Biosciences, Franklin Lakes, N.J., USA (Cytofix/Cytoperm buffer and Perm/ Wash buffer (BD biosciences reference 554714) Perm buffer III (BD biosciences reference 558050)). Phospho-STAT5 detection was measured using the AlexaFluor647 anti-human pStat5 antibody (BD biosciences reference 612599) following the manufacturer's instructions.

Probing of in vitro IL-7 dependency. For IL-7 in vitro dependency measurement, cells were cultivated in TexMacs medium (Miltenyi, Bergisch Gladbach, Germany; reference 130-097-196) with or without supplementation with anti-CD127 agents at 3 μg/mL and/or with or without rhIL-7 (AbDSerotec, Raleigh, N.C., USA; reference PHP046) at 5 ng/mL.

Assessment of in vivo efficacy of treatment through peripheral blast reduction measurement. In vivo efficacy of treatment in patient derived xenograft (PDX) mice was measured in peripheral blood samples drawn from control and treated animals on the day when control mice displayed leukemic engraftment in their blood. Blast reduction was calculated by subtracting the percentage of leukemic blasts in treated mice from the percentage of leukemic blasts detected in corresponding control animals. Briefly, the percentage of leukemic blasts in the blood of PDX mice was measured by detection of human CD45+/murine CD45−/human CD19+ (B-ALL) or human CD3+ (T-ALL) cells via flow cytometry analysis.

Standard of care in vivo treatment (overt leukemia setting). Once the leukemic engraftment was superior to 1% (day 0) as detected by flow cytometry in the blood of PDX mice, dexamethasone (70 μg orally, days 1-5); vincristine (10 μg intravenously, day 1); and PEG-asparaginase (100 IU intravenously, day 1) were administered.

Results

Anti-CD127 Agent Effect in Minimal Residual Disease Analysis in a PDX Experiment

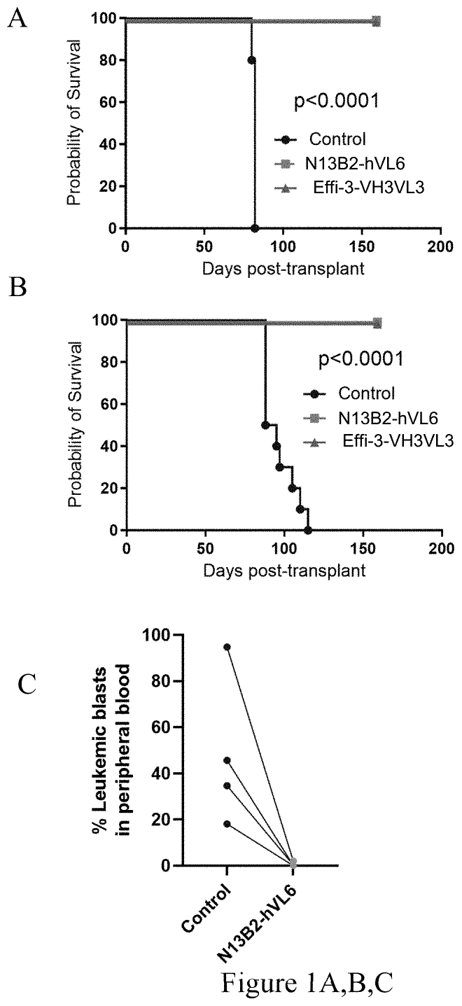

Minimal residual disease (MRD) is the name given to small numbers of leukemic cells that remain in the patient during treatment or after treatment when the patient is in remission (no symptoms or signs of disease). It is the major cause of relapse in leukemia. As illustrated on FIG. 1A, mice with a B-ALL leukemia patient-derived xenograft (PDX) treated with an anti-CD127 agent having ADCP capabilities over CD127-positive tumor cells all survive during the entire time of the experiment (160 days), while all mice that have been treated with a control were dead after 80 days post-transplantation. Further, it should be noted that 100% of mice were MRD negative, illustrating the anti-leukemic effect of the anti-CD127 agent administered to the mice, and the potential to fully and definitively treat leukemia. The same result is illustrated on FIG. 1B, wherein mice received a xenograft from another B-ALL patient. Again, it is shown that mice treated with an anti-CD127 agent having ADCP capabilities towards CD127-positive tumor cells survive, on the contrary to mice treated with a control compound, and further that most of the mice (between 80% and 90% treated with the anti-CD127 agent are MRD negative. In FIG. 1C, the impact of treatment with N13B2-hVL6 on leukemia development in four different T-ALL patient-derived xenografted mice is illustrated. Leukemic blasts are absent in the periphery of anti-CD127 treated animals, as opposed to control mice with leukemia engraftment ranging from 20 to 90% in the peripheral blood.

Effect of Administering an Anti-CD127 Agent in an Overt Leukemia Model

Overt leukemia is the setting where animals are treated when the disease is already well established in the host animal (presence of leukemic blasts over 1 to 5% in the peripheral blood).

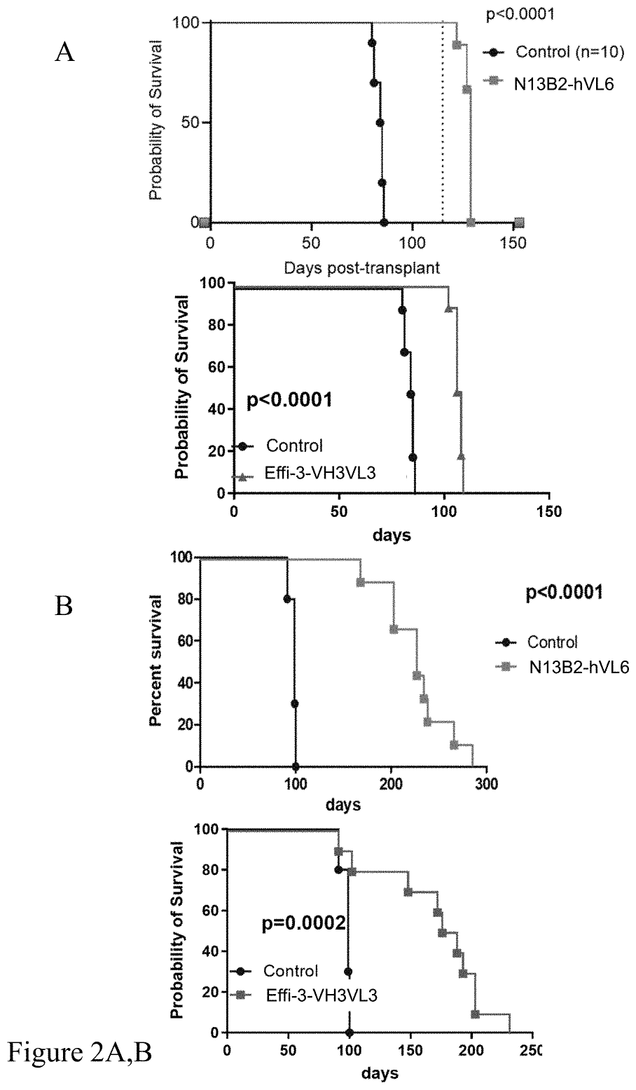

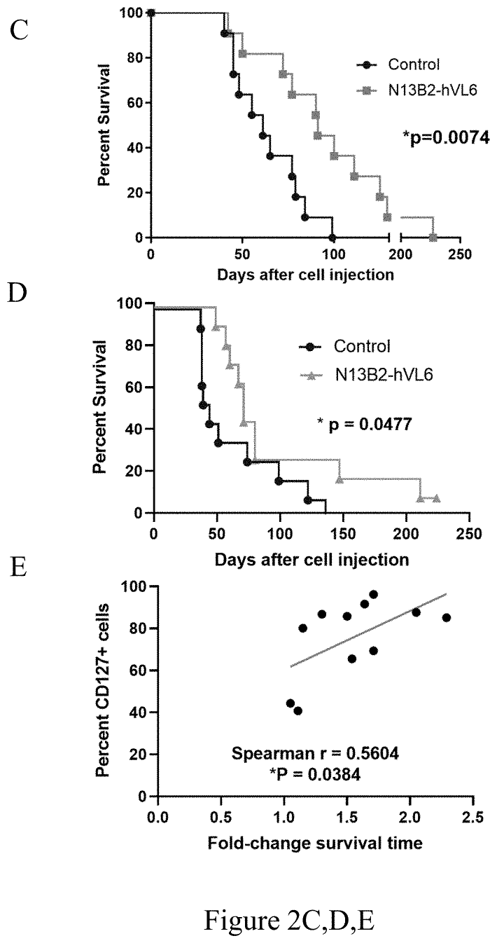

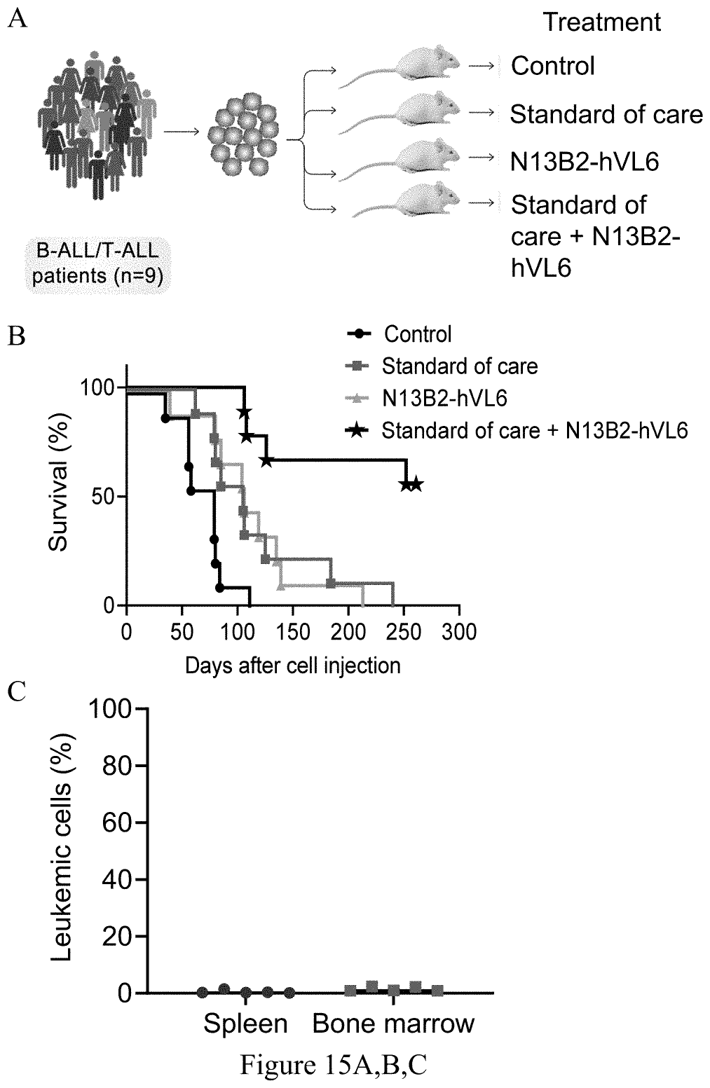

As illustrated in FIG. 2, PDX mice that have developed an overt leukemia and that were treated with an anti-CD127 agent as defined in the present application survived longer than mice treated with a control compound. In the first xenograft experiment (FIGS. 1A1 and 1A2), the mice treated with the anti-CD127 agent survived between 25% and 50% longer than mice treated with the control antibody. It should be noted that these results were obtained irrespectively of the antagonistic property of the anti-CD127 agent; indeed, even the mice treated with a neutral (i.e., not antagonistic nor agonistic) anti-CD127 agent survived longer than untreated mice. The same results have been obtained in a second experiment (FIGS. 2B1 and 2B2). In this second experiment, it can be seen that some mice treated with anti-CD127 agents that have ADCP capabilities towards CD127-positive tumor cells survived more than 200 days after transplant, twice longer than mice treated with a control. This survival rate is observed irrespectively of the antagonistic capability of the anti-CD127 agent administered to the mice; the same result is observed for mice treated with an antagonistic anti-CD127 agent (FIGS. 2A1 and 2B1) and for mice treated with a neutral (i.e., not antagonist nor agonist) anti-CD127 agent (FIGS. 2A2 and 2B2). The impact of a treatment with N13B2-hVL6 on overt leukemia development in a cohort of 12 different B-ALL patient-derived xenografted mice (FIG. 2C) and 15 different T-ALL patient-derived xenografted mice (FIG. 2D) is illustrated.

In both cases, treatment with an anti-CD127 agent significantly increased the overall survival of mice compared to control leukemic animals. In B-ALL specimens, the median overall survival was extended from 61 days to 91 days, p=0.007. In T-ALL, the median overall survival was extended from 44 days for control animals to 71 days for anti-CD127 treated animals, p=0.04). Presence of 50% or higher CD127 positive leukemic blasts in PDX specimens associated with in vivo efficacy of anti-CD127 ADCP+/ADCC− agent (FIG. 2E).

In Vitro Effect on the Phagocytosis of Tumor Cell When an Anti-CD127 Agent is Administered

As illustrated on FIG. 3, CD127 expression (assessed by specific N13B2-hVL6 binding to CD127) is variable in different examples of T-cell ALL (HPB-ALL, and CD127 mutated

DND41) and B-cell ALL (697, NAML6, and REH) cell lines, or is absent (Jurkat T-ALL cell line), as defined by using an isotype control as a negative control for the evaluation of CD127 expression level. As illustrated on FIG. 4A, wherein no cell pre-treatment has been performed, the phagocytosis of CD127 positive tumor cells is enhanced by an anti-CD127 agent, namely N13B2-hVL6 in all ALL lines expressing CD127, the level of phagocytosis achieved associating with the level of CD127 expression (probed by measurement of specific N13B2-hVL6 binding to CD127). Overall, the phagocytic index of the ADCP+/ADCC− anti-CD127 antibody N13B2-hVL6 measured on PDX ALL cells strongly correlates with its anti-leukemic in vivo efficacy as illustrated on FIG. 4B.

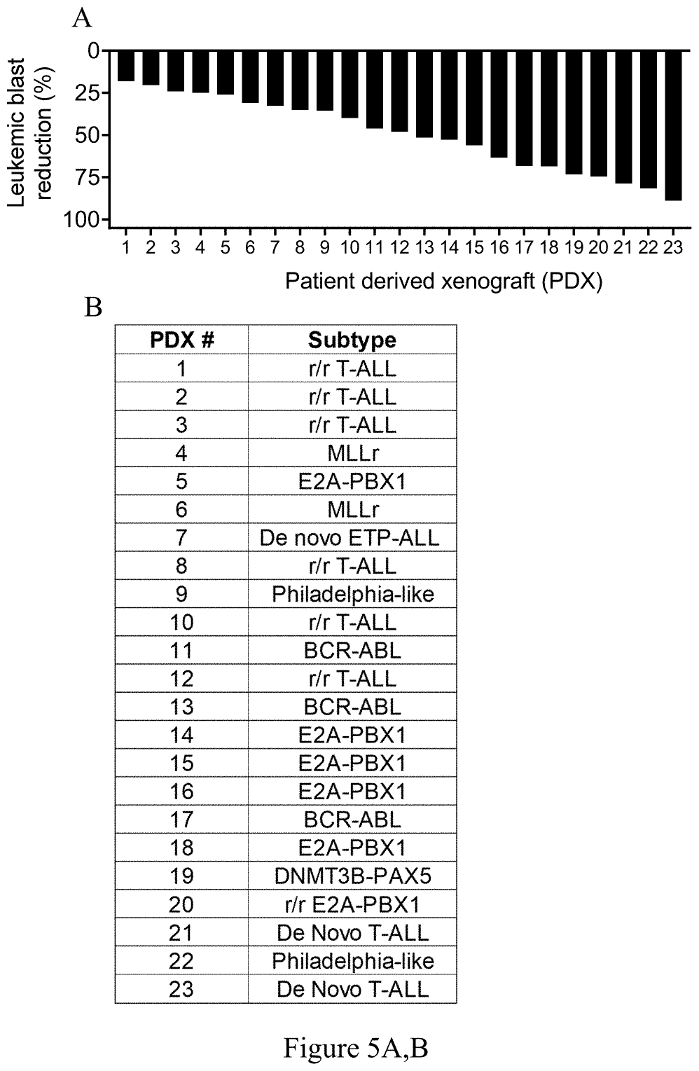

As shown on FIG. 5, the administration of N13B2-hVL6 leads to a leukemic blast reduction in 23 patients having different types of leukemias. Administering an anti-CD127 agent that has ADCP capability but no ADCC capability allows the reduction of leukemic blasts in patients having Relapsed/Refractory Acute Lymphoblastic Leukemia (r/r T-ALL), leukemia with mixed lineage leukemia mutations (MLLr), ALL with mutated E2A-Pbxl, early T-cell precursor (ETP) ALL, Philadelphia-like ALL, ALL with mutated bcr, B-ALL with pax5 alterations.

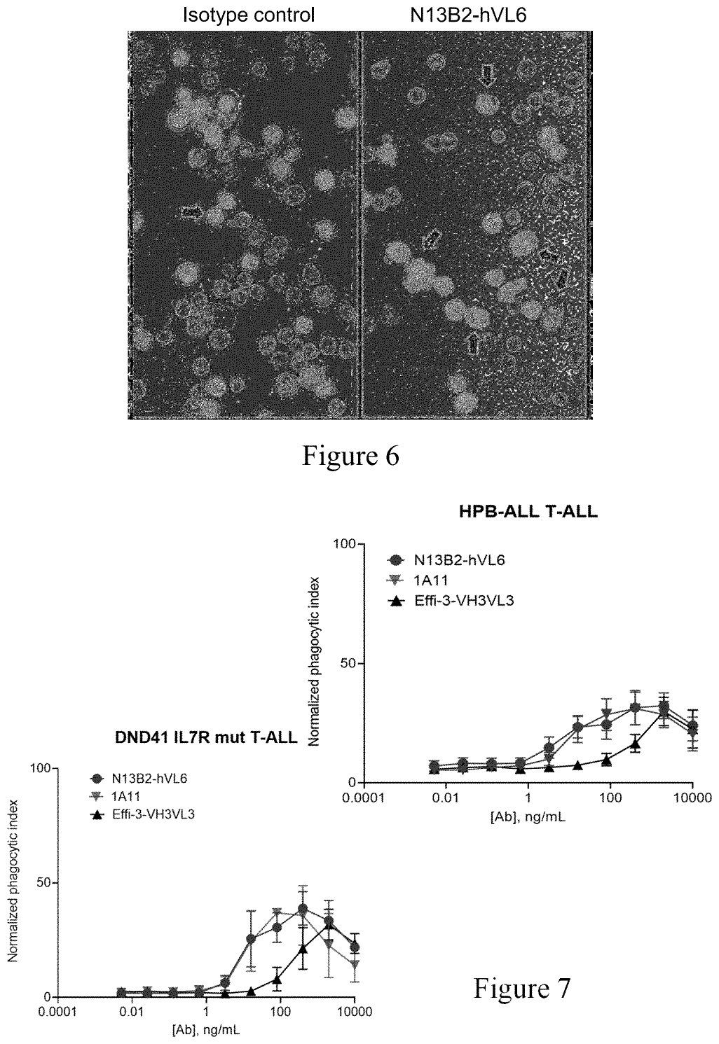

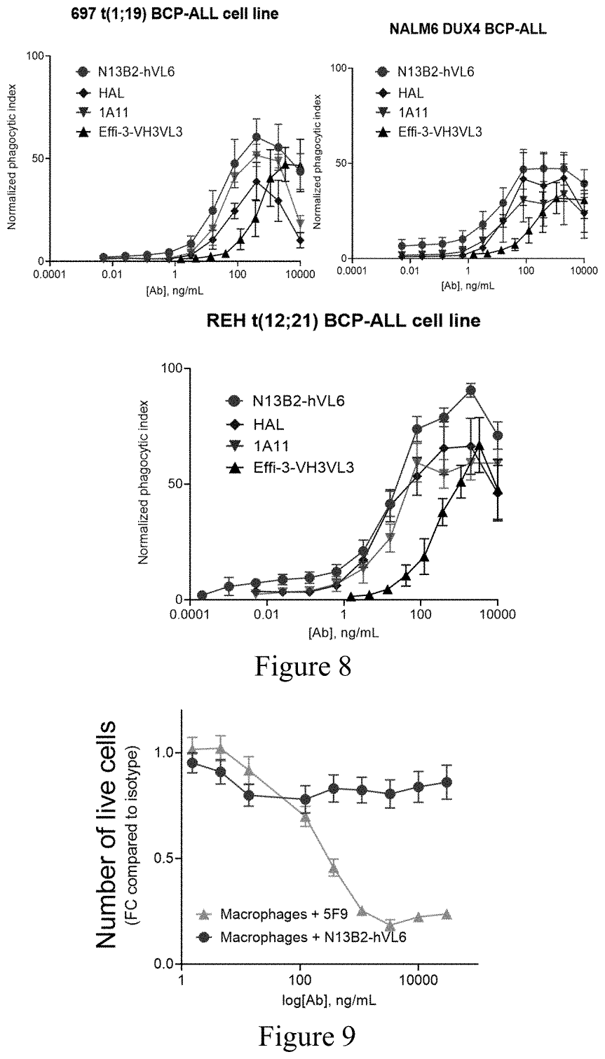

As shown on FIG. 6, the administration of N13B2-hVL6 leads to the phagocytosis of leukemia cells by macrophages, illustrating the positive effect of the anti-CD127 agent to induce, sustain, or enhance the phagocytosis of tumor cells. On FIGS. 7 and 8, the capability of several different anti-CD127antibodies to enhance phagocytosis of tumor cells issued from five ALL cell lines expressing CD127 has been tested. Four anti-CD127 antibodies, namely N13B2-hVL6 (with ADCP capability but no ADCC capability: ADCP+/ADCC-), EFFI-3-VH3VL3 (both in-house antibodies), HAL (initially designed by Pfizer and produced in-house) and 1A11 (initially designed by GlaxoSmithKline and produced in-house) all three having ADCP and ADCC capabilities (ADCP+/ADCC+) have been administered at increasing doses on two T-cell ALL cell lines (HPB-ALL and DND41 IL7R mut.) and three B-ALL cell lines (697 t(1;19), NAML6 (DUX4) and REH t(12;21)) in presence of human macrophages. The phagocytosis of tumor cells by macrophages has been assessed according to the method described here above. On FIG. 7, it is shown that all three anti-CD127 antibodies enhance the phagocytosis of T-ALL tumor cells by macrophages. While it may be considered that the anti-CD127 antibody EFFI-3-VH3VL3 is less efficient to enhance the phagocytosis of tumor cells by macrophages, it should be noted that this antibody is less affine for its target CD127 than the other tested antibodies. It can be seen that the anti-CD127 antibody N13B2-hVL6 is very efficient in enhancing the phagocytosis of tumor cells by macrophages. Similar results are illustrated on BALL-cell lines on FIG. 8. The anti-CD127 agents are all able to enhance the phagocytosis of B-ALL tumor cells by macrophages. The anti-CD127 antibody N13B2-hVL6 is the most efficient to enhance the phagocytosis of B-ALL tumor cells.

To sum up, these results illustrate that while all anti-CD127 agents tested are efficient to enhance the phagocytosis of CD127-positive tumor cells by macrophages through the ADCP mechanism of action, irrespectively of the type of ALL, including CD127 mutated ALL, N13B2-hVL6 (ADCP+ADCC−) demonstrated the strongest ADCP capability against CD127-positive tumor cells by macrophages, at levels that can surpass that of the reference anti-CD47 antibody 5F9 antibody (see FIG. 10).

Lack of In Vitro Toxicity Effect Against Macrophages and Healthy T Cells when an Anti-CD127 Agent of the Invention is Administered, and Lack of ADCC Activity Against Hhuman CD127+Cells, in Particular Against Human Immune Cells (in Particular Against Human T Cells)

The toxicity (i.e., deleterious effect like cell apoptosis or other mechanisms leading to the loss of viable cells) of an anti-CD127 antibody (N13B2-hVL6) or an anti-CD47 antibody (5F9) against macrophages has been assessed and the results are illustrated on FIG. 9. As illustrated, the overall number of live macrophages is not impacted by the dose of anti-CD127 antibody added; it means that the anti-CD127 antibody does not lead to a reduction of the overall number of macrophages, irrespectively of its dosage. On the contrary, when an anti-CD47 agent is administered, the overall number of macrophages is drastically reduced with the administered doses, suggesting that the anti-CD47 agent has a toxic effect on macrophages that leads to their depletion.

According to these results, the ADCP+/ADCC− N13B2-hVL6 antibody does not have a negative impact on the overall population of macrophages and does not have any adverse effect on their capability to phagocytose tumor cells, unlike other agents currently used in the treatment of ALL.

The phagocytosis of tumor cells (from the REH cell line) and normal T cells by macrophages has been assessed in presence of an ADCP+/ADCC- anti-CD127 antibody (N13B2-hVL6) and an anti-CD47 antibody (5F9), which is a positive control for potent induction of phagocytosis. The results are illustrated on FIG. 10. The anti-CD127 antibody does not have any significant impact on the phagocytosis of normal T cells by macrophages. A similar result is obtained when an anti-CD47 agent is administered. However, the administration of an ADCP+/ADCC− anti-CD127 antibody (N13B2-hVL6) leads to a higher increase in phagocytosis of the tumor cells than that of an anti-CD47 agent. These results mean that the anti-CD127 agents are more likely to enhance the phagocytosis of tumor cells while leaving the normal T cells unharmed at different doses. By combining the results illustrated on FIGS. 8 and 9, the inventors show for the first time that the ADCP+/ADCC− anti-CD127 agents of the invention do not lead to macrophage depletion, do not lead to healthy T cell phagocytosis, while they greatly enhance CD127-positive tumor cells phagocytosis by macrophages. These results are further confirmed by the data illustrated on FIG. 11A, which correspond to the phagocytosis of macrophages by macrophages (termed here “autophagocytosis”) in presence of an ADCP+/ADCC− anti-CD127 antibody (N13B2-hVL6) or an anti-CD47 antibody (5F9). In presence of the anti-CD47 antibody, the macrophages have an autophagocytosis activity, due to the expression of CD47 by macrophages. When the anti-CD127 antibody is administered, there is no autophagocytosis of macrophages. These results clearly illustrate once again the lack of toxicity of the anti-CD127 antibody. Further, the ADCC against human CD127+ cells, in particular against immune cells (T cells), by Natural Killer cells induced by the anti-CD127 agent used in the invention has been assessed. The results are illustrated on FIGS. 11B and 11C. As illustrated, the anti-CD127 agents with ADCP capabilities and which do not induce ADCC do not lead to cytotoxicity, in particular to lymphodepletion, (no cytotoxicity against immune cells (T cells)), contrary to the positive control, which is an antibody that binds to the same target CD127, but which is known for enhancing ADCC activity.

Lack of In Vivo Toxicity Against Healthy Lymphocytes in Humans when an Anti-CD127 Agent is Administered

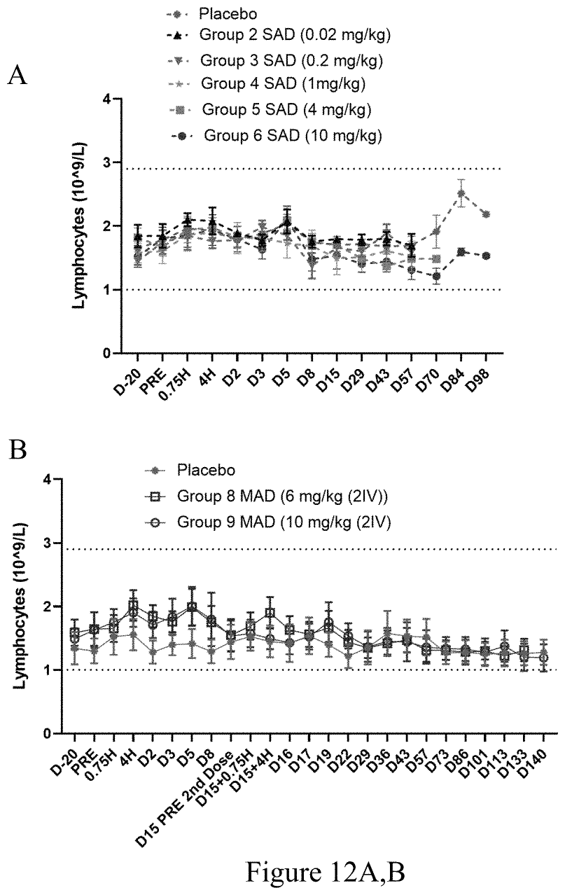

The toxicity (i.e., deleterious effect like cell apoptosis or other mechanisms leading to the loss of viable cells) of an ADCP+/ADCC− anti-CD127 antibody (N13B2-hVL6) against human lymphocytes in vivo has been assessed during a phase 1 clinical trial (EUDRACT number 2018-001832-22) and the results are illustrated on FIG. 12. The administration of single dose of N13B2-hVL6 (0.002, 0.02, 0.2, 1, 4, or 10 mg/kg IV or 1 mg/kg SC) or two doses given 2 weeks apart (6 or 10 mg/kg) was safe and well-tolerated. In all subjects exposed to N13B2-hVL6 up to 10 mg/kg (single and double doses) no clinically significant lymphopenia was reported after N13B2-hVL6 administration.

Effect of Administering an Anti-CD127 Agent Alone or in Combination in a CD127 Positive ALL Cell Line

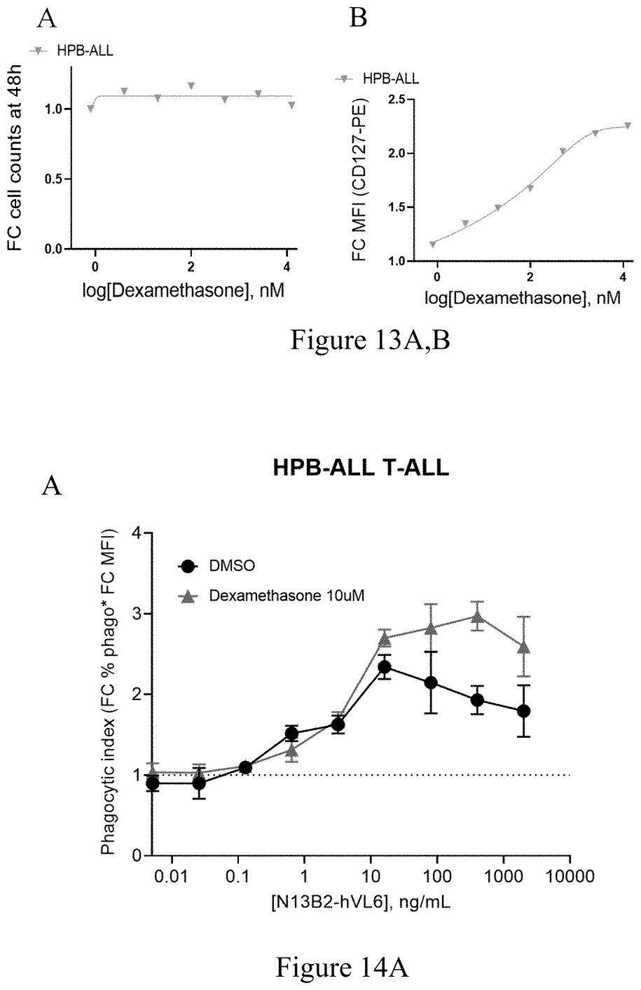

As discussed in the description of the invention, several forms of leukemias are resistant to current treatment. As an example, dexamethasone is used to treat different forms of leukemias, but several T-cell ALL and B-cell ALL are known to be resistant to dexamethasone, such as the HPB-ALL cell line (see FIG. 13A) Interestingly, in response to increasing concentrations of dexamethasone treatment, CD127 expression was increased in this cell line in a dose dependent fashion (FIG. 13B).

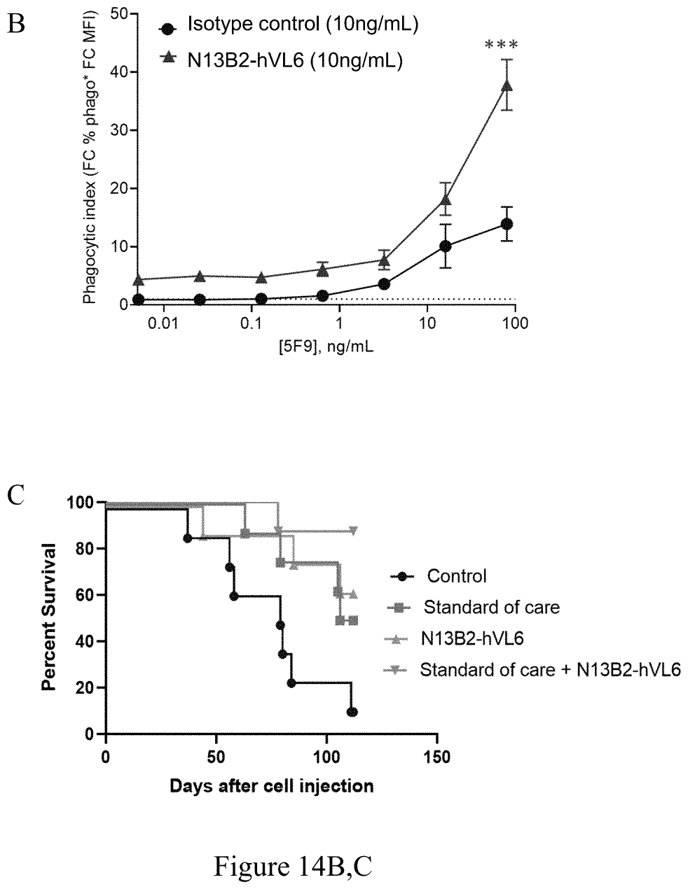

The ADCP+/ADCC− anti-CD127 antibody N13B2-hVL6 has been administered to the T-ALL HPB-ALL cell line in presence or absence of dexamethasone. As illustrated in FIG. 14A, a synergetic effect of the combination of the anti-CD127 N13B2-hVL6 antibody and dexamethasone can be observed in HPB-ALL cells. These results mean that the use of anti-CD127 agents with ADCP+/ADCC− capabilities is efficient to enhance the phagocytosis of tumor cells by macrophages and may be efficient to treat CD127-positive cancer, but it also means that these ADCP+/ADCC− anti-CD127 agents may be useful to treat patient that have a CD127-positive cancer that is resistant to current therapies, like dexamethasone therapy.

The ADCP+/ADCC− anti-CD127 antibody N13B2-hVL6 has been administered to the B-ALL NALM6 cell line in presence or absence of the anti-CD47 antibody Magrolimab (5F9). As illustrated in FIG. 14B, a synergetic effect of the combination of the anti-CD127 N13B2-hVL6 antibody and anti-CD47 antibody can be observed in NALM6 cells. These results mean that the use of anti-CD127 agents with ADCP+/ADCC− capabilities is efficient to enhance the phagocytosis of tumor cells by macrophages and may be efficient to treat CD127-positive cancer, but it also means that these ADCP+/ADCC− anti-CD127 agents may be useful to treat patient that have a CD127-positive cancer in combination with other phagocytosis-enhancing agents such as anti-CD47 agents.

Furthermore, in a cohort of 8 ALL specimens, a synergistic efficacy was observed in overt leukemia PDX setting between ADCP+/ADCC− anti-CD127 antibody N13B2-hVL6 treatment and the standard of care (SOC) treatment in ALL (SOC comprising the administration of Dexamethasone+Oncaspar (known under references Pegaspargase/PEG-Asparaginase)+Vincristine), as shown in FIG. 14C.

FIG. 15 illustrates in vivo results obtained with xenografts issued from 9 leukemic patients. 4 differents treatmtents were administered to mice with patient-derived xenografts: control, the standard of care for treating ALL ((vincristine, PEG-asparaginase and dexamethasone), the anti-CD127 agent N13B2-hVL6, and the combination of the standard of care with the anti-CD127 agent N13B2-hVL6 (FIG. 15A). As illustrated in FIG. 15B, more than 50% of the mice (5/9) were alive 260 days after the injection of the combination of the standard of care with the anti-CD127 agent N13B2-hVL6, when all mice injected with control, the standard of care or the anti-CD127 agent N13B2-hVL6 did not survive. It is therefore established that the combination of the standard of care for treating ALL (vincristine, PEG-asparaginase and dexamethasone) and the anti-CD127 agent N13B2-hVL6 is very effective for treating ALL, with an improved overall survival. As shown in FIG. 15C, leukemic blasts in mice treated with and the combination of the standard of care and the anti-CD127 agent N13B2-hVL6 that survived more than 260 days were not detected in the bone marrow or the spleen, illustrating the remission of the disease in these mice.

Effect on the Phagocytosis of Tumor Cells by Macrophage in Presence of Different Anti-CD127 Agents

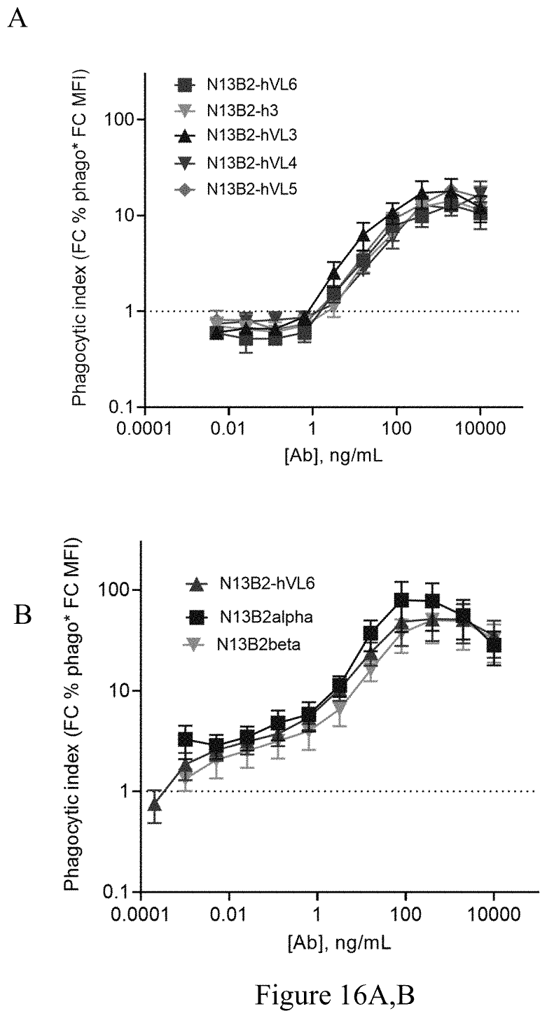

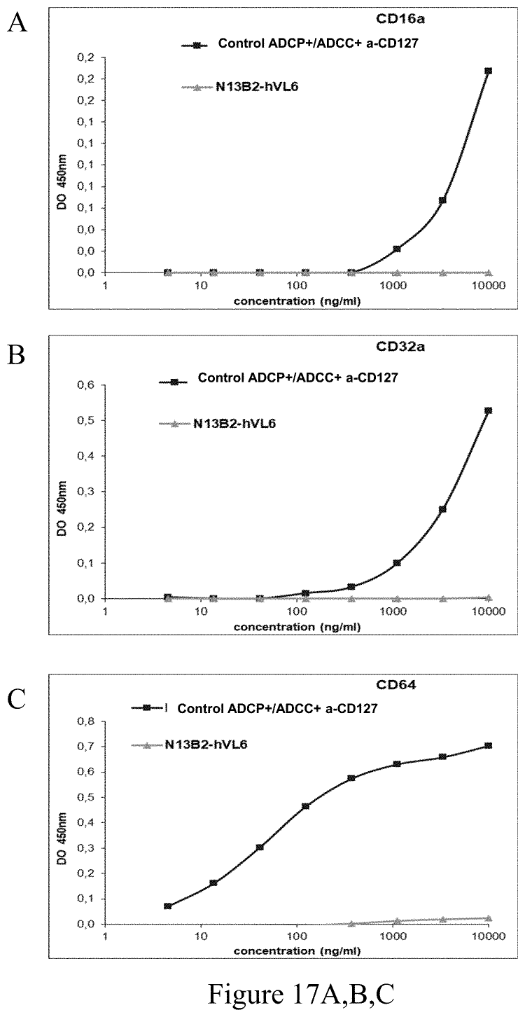

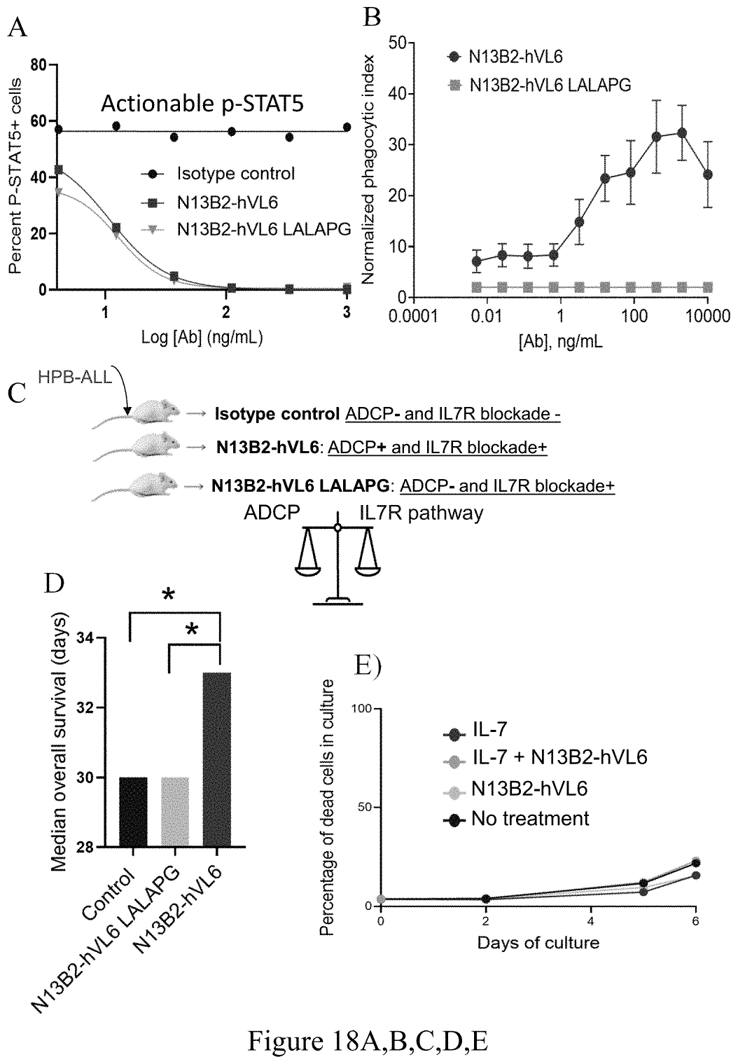

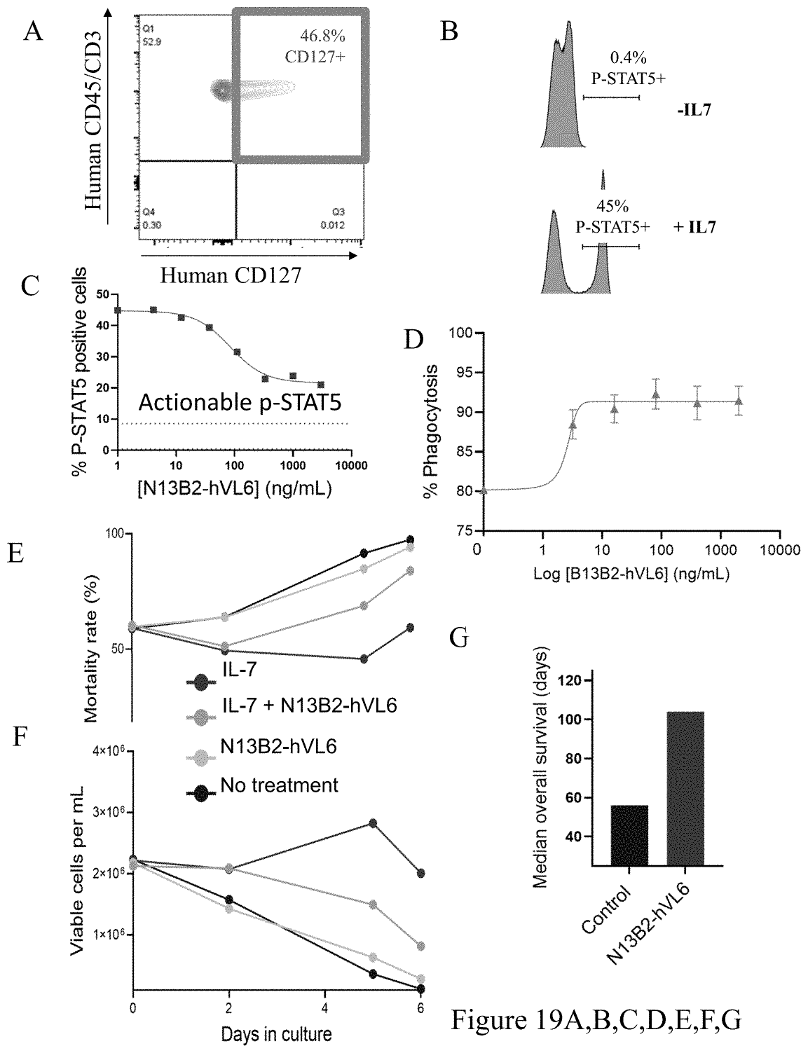

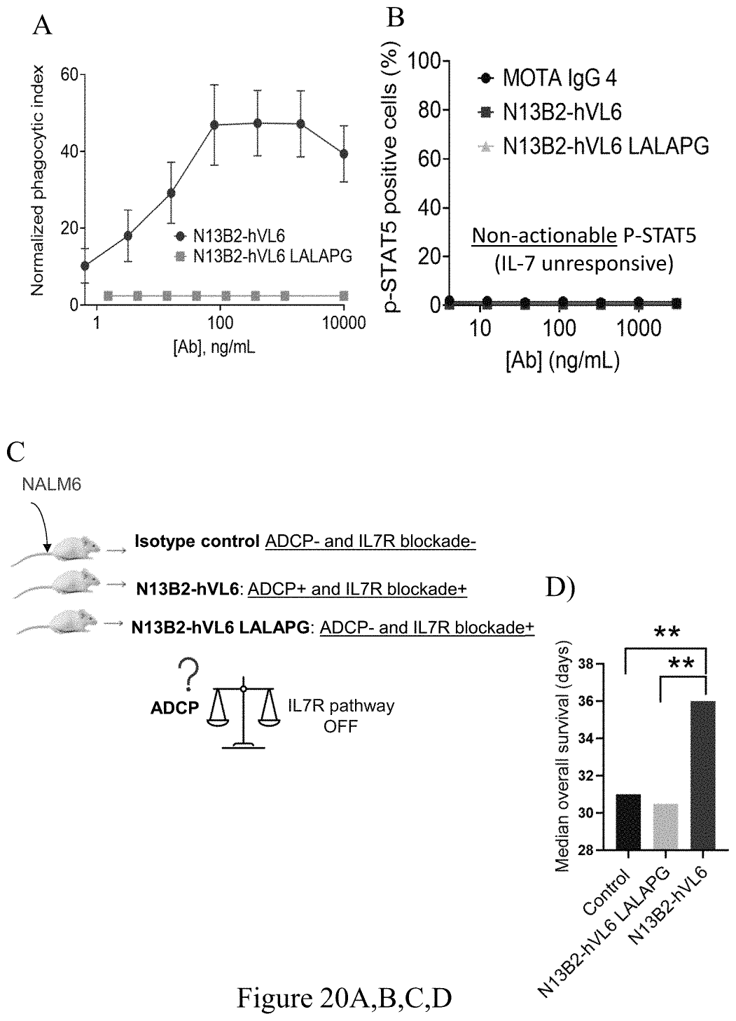

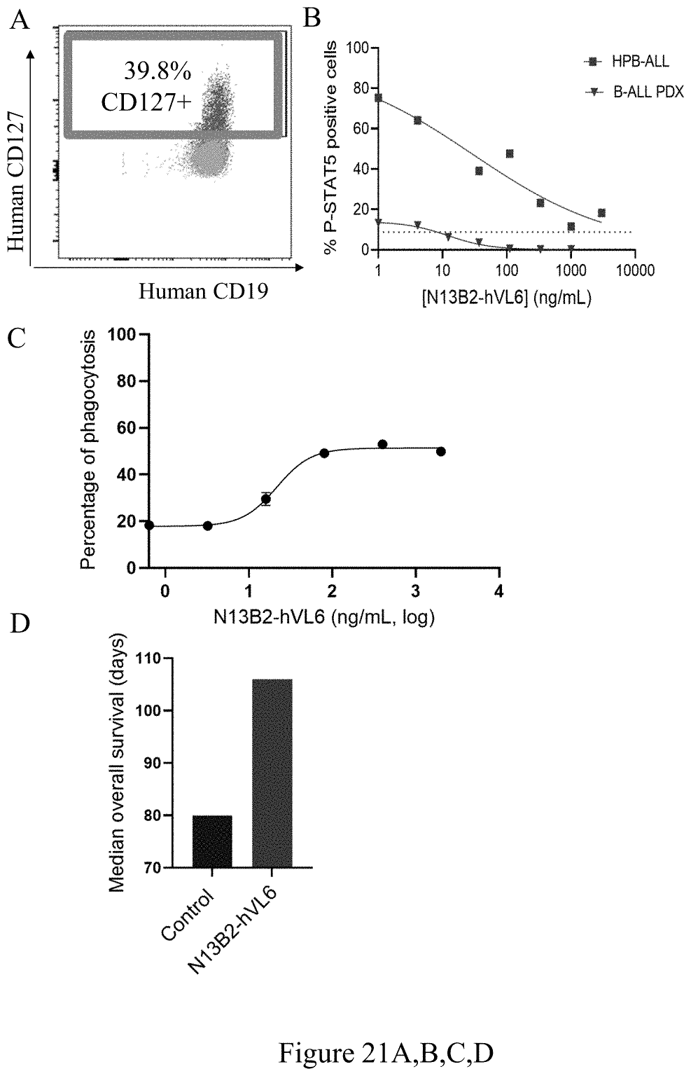

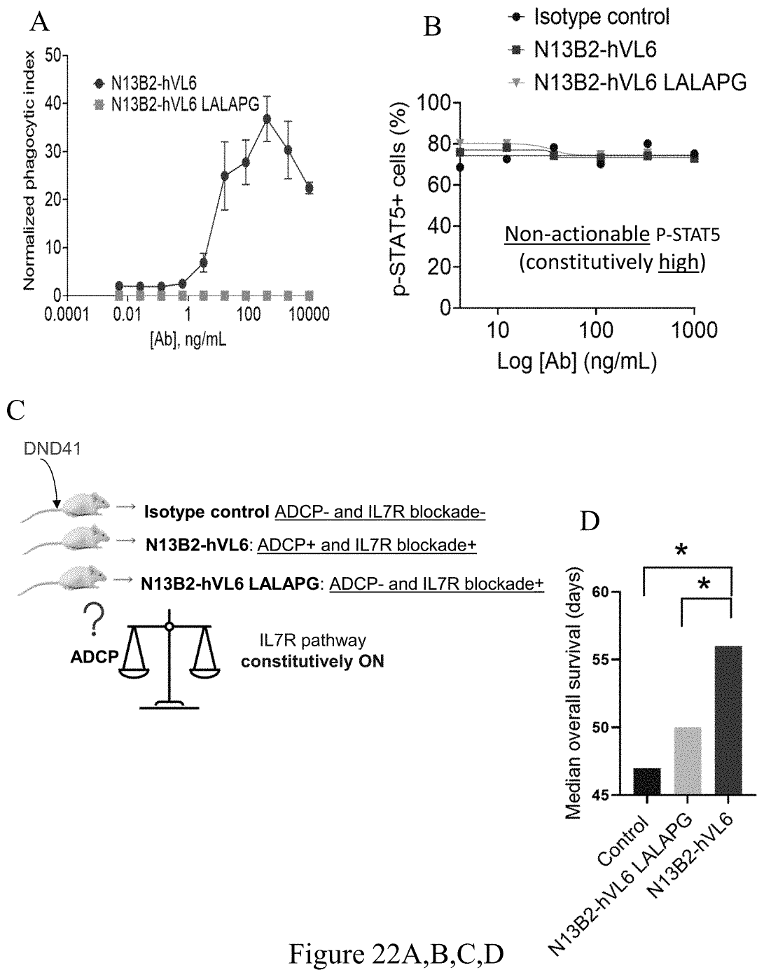

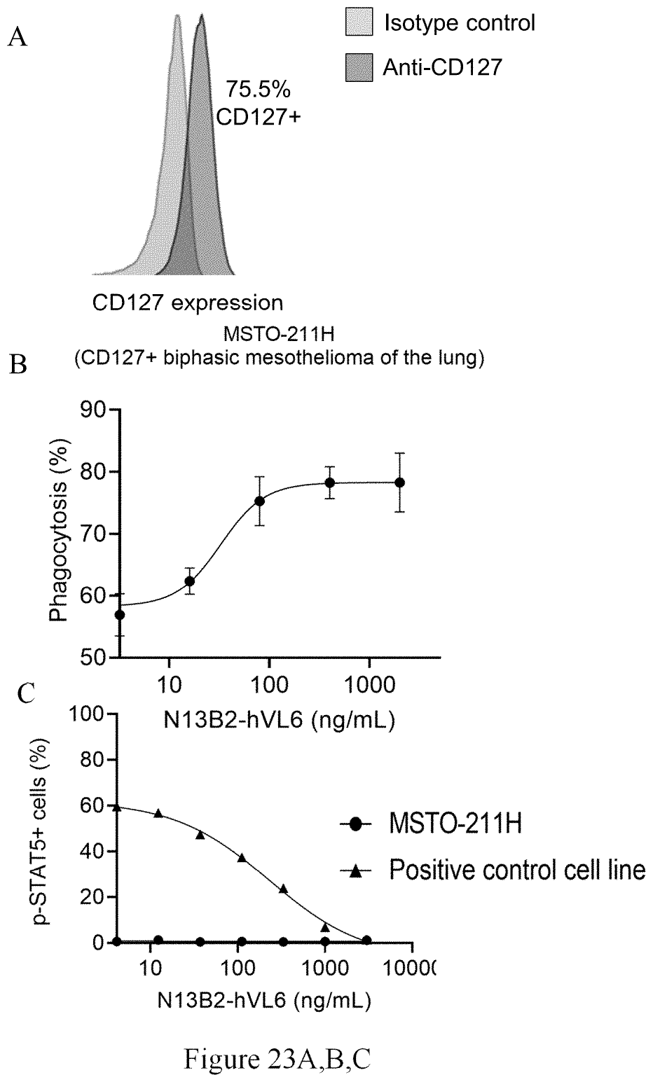

Several anti-CD127 antibodies corresponding to their definition in the description of the invention have been tested to assess their capabilities to enhance the phagocytosis of tumor cells by macrophages. As illustrated on FIG. 16A , anti-CD127 antibodies which share the same CDR domains but different frameworks and on FIG. 16B, anti-CD127 antibodies which share close related CDR domains (with only one or two mutations within the 6 CDRs domains) have all the same capability to enhance the phagocytosis of tumor cells by macrophages.

Effect on the Binding to FCγR of Different Anti-CD127 Agents

Investigation of N13B2-hVL6 to main activating FCγR by ELISA technology (FIG. 17) indicates that, contrary to a positive control ADCP+/ADCC+ anti-CD127 antibody, N13B2-hVL6 does not bind efficiently to CD16a (FIG. 17A), CD32a (FIG. 17B) or CD64 (FIG. 17C), further highlighting the unexpected capacity of N13B2-hVL6 to induce robust ADCP in CD127 positive tumor cells. These three receptors bind antibodies through the Fc domain of the antibodies, thereby inducing ADCC and ADCP. As illustrated, the antibody that has an IgG1 domain binds to these three receptors, which was intended, and may accordingly induce cell clearance mechanisms through ADCC and ADCP. But the anti-CD127 antibodies that are IgG4 do not bind to these receptors, which can explain the lack of ADCC capability of these antibodies. Nonetheless, the ADCP capabilities of these antibodies are thus unexpected, because ADCP mechanism is especially mediated by these three receptors.

Anti-Leukemic Mechanism of Action of ADCP+/ADCC− Anti-CD127 Agents in the Treatment of Diverse Subtypes of CD127 Positive Leukemias

In order to evaluate the importance of the ADCP mechanism of action for the anti-leukemic activity of ADCP+/ADCC− anti-CD127 agents, we interrogated a series of diverse ALL cell lines and patient derived xenografted cells reflecting the complexity of situations observed in the clinic: for example, a) presence of an actionable IL-7R pathway without dependency (FIG. 18) or with dependency (FIG. 19) on the IL-7R pathway for cell survival and proliferation, b) presence of a non-actionable pathway (absence, FIG. 20, or poor, FIG. 21, p-STAT5 signaling upon IL-7 stimulation or activating mutation of the IL-7R leading to a constitutive activation of the pathway (FIG. 22), a frequent phenomenon in T-ALL). To interrogate the importance of ADCP on the in vivo efficacy of the ADCP+/ADCC N13B2-hVL6 anti-CD127 agent, we generated an Fc-DEAD-DEAD variant through introduction of the LALAPG mutation (Lo et al. J Biol Chem. 2017) (referenced N13B2-hVL6 LALAPG in the present description and in the figures). This mutation precludes N13B2-hVL6 LALAPG antibody-mediated effector functions such as ADCP and ADCC, on the contrary to N13B2-hVL6, while preserving the N13B2-hVL6 blocking activity on the IL-7R signaling pathway, as illustrated in FIG. 18A throught the measurement of p-STAT5 concentration in presence of increasing concentratiosn of the antibodies N13B2-hVL6 and N13B2-hVL6 LALAPG.

The ADCP+/ADCC− anti-CD127 antibodies N13B2-hVL6 and N13B2-hVL6 LALAPG can efficiently inhibit CD127 signaling (p-STATS, FIG. 18A) in the IL-7 responsive T-ALL cell line HPB-ALL, whereas only N13B2-hVL6 is able to induce ADCP in these cells (FIG. 18B). In vivo interrogation of this ADCP+/IL-7 responsive model (FIG. 18C) shows antileukemic activity of N13B2-hVL6, as opposed to its Fc-DEAD-DEAD LALAPG-variant FIG. 18D, indicating that the ADCP mechanism of action is the main driver of anti-leukemic activity in this IL-7 responsive ALL. The HPB-ALL cell line is however not dependent on IL-7 for its survival in vitro FIG. 18E. A patient derived T-ALL specimen expressing CD127 (FIG. 19A) is able to induce p-STAT5 signaling upon IL-7 stimulation (FIG. 19B). The ADCP+/ADCC− anti-CD127 antibody N13B2-hVL6 can efficiently inhibit CD127 signaling in this patient's cells (p-STATS, FIG. 19C) and induce ADCP (FIG. 19D). This T-ALL PDX specimen is dependent on IL-7 for survival and proliferation in vitro (FIG. 19E-F) and in this context, the ADCP+/ADCC− anti-CD127 antibody N13B2-hVL6 exerts anti-leukemic activity in vivo FIG. 19G. The ADCP+/ADCC- anti-CD127 antibody N13B2-hVL6 induces ADCP (FIG. 20A) but cannot inhibit CD127 signaling (p-STATS, FIG. 20B) in the IL-7 unresponsive B-ALL cell line NALM6. However, in vivo interrogation of this ADCP+/IL-7 unresponsive model (FIG. 20C) shows antileukemic activity of N13B2-hVL6, as opposed to N13B2-hVL6 LALAPG, FIG. 20D, indicating that the ADCP mechanism of action alone is able to exert anti-leukemic activity. A patient derived B-ALL specimen expressing CD127 (FIG. 21A) is very weakly able to induce p-STATS signaling upon IL-7 stimulation (FIG. 21B), but ADCP is induced efficiently by N13B2-hVL6 treatment in these leukemic cells (FIG. 21C). In this context, the ADCP+/ADCC- anti-CD127 antibody N13B2-hVL6 exerts anti-leukemic activity in vivo FIG. 21D.The ADCP+/ADCC- anti-CD127 antibody N13B2-hVL6 induces ADCP (FIG. 22A) but cannot inhibit CD127 signaling (p-STATS, FIG. 22B) in the IL-7R mutated, consitutively activated T-ALL cell line DND41. However, in vivo interrogation of this ADCP+/IL-7R constitutively activated model (FIG. 22C) shows antileukemic activity of N13B2-hVL6 as opposed to its Fc-DEAD-DEAD LALAPG-variant FIG. 22D, indicating that the ADCP mechanism of action alone is able to exert anti-leukemic activity even in the context of IL-7R activating mutations (which are frequent in T-ALL).

Further, the effect of the anti-CD127 antibody N13B2-hVL6 on the phagocytosis of human malignant mesothelioma cells expressing CD127 (FIG. 23A) and issued from pleura has been assessed. As illustrated in FIG. 23B, administering increasing doses of the ADCP+/ADCC− anti-CD127 antibody enhances the phagocytosis of CD127+ human mesothelioma cells. Interestingly, these human mesothelioma cells are unable to induce p-STAT5 signaling upon IL-7 stimulation, indicating a dysfunctionality of the IL-7R signaling-pathway (FIG. 23C),

These experiments confirm the efficacy of the ADCP+/ADCC- anti-CD127 agent of the invention for treating CD127+ cancers, in particular when the IL-7R signaling pathway is dysfunctional by enhancing the phagocytosis of tumor cells expressing CD127.

Certain Embodiments of the Present Disclosure can be Defined in Any of the Following Numbered Paragraphs:

Paragraph 1. An anti-CD127 agent, in particular an anti-CD127 antibody or antigen-binding fragment thereof or antigen-binding antibody mimetic, for use as a medicament in the treatment of a patient having a CD127-positive cancer, in particular by enhancing the phagocytosis of CD127-positive tumor cells, more particularly by macrophages, wherein the anti-CD127 agent has Antibody Dependent Cellular Phagocytosis (ADCP) activity against CD127-positive tumor cells, in particular by macrophages, and does not have Antibody Dependent Cytotoxic Activity (ADCC), in particular against immune cells, more particularly against T cells.

Paragraph 2. The anti-CD127 agent according to paragraph 1 for use according to paragraph 1, wherein the CD127-positive cancer is Leukemia, in particular is Acute Lymphoblastic Leukemia (ALL), more particularly is T-cell ALL or B-cell ALL.

Paragraph 3. The anti-CD127 agent according to paragraph 1 or 2 for use according to paragraph 1 or 2, wherein the CD127-positive cancer is selected from the group consisting of CD127 overexpressing Acute Lymphoblastic Leukemia (ALL), CD127 and/or JAK-STAT pathway mutated ALL, BCR-ABL1—like ALL, and B cell precursor ALL bearing one the following cytogenetics: t(1;19), t(12,21), MLL-rearrangements, hyperdiploid karyotypes, trisomy 4 and trisomy10.

Paragraph 4. The anti-CD127 agent according to any one of paragraphs 1 to 3 for use according to any one of paragraphs 1 to 3, wherein the CD127-positive cancer is a solid cancer, in particular is mesothelioma.

Paragraph 5. The anti-CD127 agent according to any one of paragraphs 1 to 4 for use according to any one of paragraphs 1 to 4, wherein the CD127-positive cancer is treated by the phagocytosis of CD127-positive tumor cells, in particular by macrophages.

Paragraph 6. The anti-CD127 agent according to any one of paragraphs 1 to 5 for use according to any one of paragraphs 1 to 5, wherein the anti-CD127 agent does not induce lymphodepletion in said patient.

Paragraph 7. The anti-CD127 agent according to any one of paragraphs 1 to 6 for use according to any one of paragraphs 1 to 6, wherein the anti-CD127 agent is an anti-CD127 antibody or antigen-binding fragment thereof, comprising a constant chain belonging to the subclass of IgG1, IgG2, IgG3 or IgG4, in particular the subclass of mammalian IgG1, IgG2, IgG3 or IgG4, more particularly the subclass of mammalian IgG4.

Paragraph 8. The anti-CD127 agent according to any one of paragraphs 1 to 7 for use according to any one of paragraphs 1 to 7, wherein the anti-CD127 agent is selected from the group consisting of chimeric antibodies, humanized antibodies and fully human monoclonal antibodies.

Paragraph 9. The anti-CD127 agent according to any one of paragraphs 1 to 8 for use according to any one of paragraphs 1 to 8, wherein the anti-CD127 agent is an anti-CD127 antibody or antigen-binding fragment thereof, which comprises:
a VH chain comprising at least the following amino acid sequences:
   VHCDR1 SEQ ID No. 3;
   VHCDR2 SEQ ID No. 4;
   VHCDR3 SEQ ID No. 5 or SEQ ID No. 6;
and a VL chain comprising at least the following amino acid sequences:
   VLCDR1 SEQ ID No. 7 or SEQ ID No. 8;
   VLCDR2 SEQ ID No. 9 or SEQ ID No. 10;
   VLCDR3 SEQ ID No. 11.

Paragraph 10. The anti-CD127 agent according to any one of paragraphs 1 to 9, for use according to any one of paragraphs 1 to 9, wherein the anti-CD127 antibody or antigen-binding fragment thereof is an antagonist of the IL-7R signaling pathway induced by the binding of IL-7 to CD127.

Paragraph 11. The anti-CD127 agent according to any one of paragraphs 1 to 10, for use according to any one of paragraphs 1 to 10, wherein the anti-CD127 agent is used in combination with a conventional treatment of cancer.

Paragraph 12. The anti-CD127 agent according to any one of paragraphs 1 to 11, for use according to any one of paragraphs 1 to 11, in combination with at least one second therapeutic agent selected from the group consisting of a chemotherapeutic agent, a targeted cancer therapy agent, an immunotherapeutic agent and a radiotherapy agent, in particular for a simultaneous, separate or sequential use.

Paragraph 13. The anti-CD127 agent according to paragraph 12 for use according to paragraph 12, wherein the at least one second therapeutic agent is selected from the group consisting of a cytotoxic agent, a chemotherapeutic agent, an anti-angiogenic agent, a cell-cycle control/apoptosis regulating agent, a hormonal regulating agent and an anti-cancer immunogen agent, in particular an anti-cancer antibody, more particularly a tumor-targeting antibody.

Paragraph 14. The anti-CD127 agent according to any one of paragraphs 1 to 13 for use according to any one of paragraphs 1 to 13, in combination with at least one second therapeutic agent selected from the group consisting of an anti-CD3 agent, in particular anti-CD3 antibody, an anti-PD1 agent, in particular an anti-PD1 antibody, an anti-PDL1 agent, in particular an anti-PDL1 antibody, an anti-CTLA4 agent, in particular an anti-CTLA4 antibody, an agonist of CD137, in particular an agonist anti-CD137 antibody, an anti-VEGF agent, in particular an anti-VEGF antibody, an anti-CLEC-1 agent, in particular an anti-CLEC-1 antibody, an anti-CD28 agent, in particular an anti-CD28 antibody, an anti-CD19 agent, in particular an anti-CD19 antibody, and anti-CD47 agent, in particular an anti-CD47 antibody, an anti-SIRPa agent, in particular an anti-SIRPa antibody, an anti-Bcl-2 agent, in particular venetoclax, an inhibitor of the tyrosine/kinase pathway, Dexamethasone, rituximab, trastuzumab, cetuximab, Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Vincristine, Marqibo (Vincristine Sulfate Liposome); Mercaptopurine (or Purinethol or Purixan); Methotrexate Sodium (or Trexall); Nelarabine; Oncaspar (or Pegasparagase or PEG-Asparaginase); Ponatinib Hydrochloride; Prednisone; Purinethol (Mercaptopurine); Vincristine Sulfate, Vincristine Sulfate Liposome, in particular for a simultaneous, separate or sequential use.

Paragraph 15. The anti-CD127 agent according to paragraph 14 for use according to paragraph 14, wherein the at least one second therapeutic agent is Dexamethasone and/or Oncaspar (or Pegaspargase or PEG-Asparaginase) and/or Vincristine, in particular Dexamethasone and Oncaspar (or Pegaspargase or PEG-Asparaginase) and Vincristine.

Paragraph 16. The anti-CD127 agent according to any one of paragraphs 1 to 15, for use according to any one of paragraphs 1 to 15, wherein said anti-CD127 agent is administered to said patient after the patient has been evaluated as having CD127-positive tumor cells.

Paragraph 17. An anti-CD127 agent for use as a medicament for promoting phagocytosis of CD127-positive tumor cells of a patient, in particular by macrophages, wherein the anti-CD127 agent has Antibody Dependent Cellular Phagocytosis (ADCP) activity against CD127-positive tumor cells, in particular by macrophages, and does not have Antibody Dependent Cytotoxic Activity (ADCC), in particular against immune cells, more particularly against T cells.

Paragraph 18. The anti-CD127 agent according to paragraph 17 for use according to paragraph 17, wherein said patient has a CD127-positive cancer selected from the group consisting of Leukemia, in particular Acute Lymphoblastic Leukemia (ALL), more particularly T-cell ALL or B-cell ALL, and solid cancers, in particular mesothelioma.

Paragraph 19. The anti-CD127 agent according to paragraph 17 or 18 for use according to paragraph 17 or 18, in combination with a conventional treatment of cancer.

Paragraph 20. The anti-CD127 agent according to any one of paragraphs 17 to 19 for use according to any one of paragraphs 17 to 19, in combination with at least one second therapeutic agent selected from the group consisting of a chemotherapeutic agent, a targeted cancer therapy agent, an immunotherapeutic agent a and radiotherapy agent, in particular for a simultaneous, separate or sequential use.

Paragraph 21. The anti-CD127 agent according to any one of paragraphs 17 to 20 for use according to any one of paragraphs 17 to 20, wherein the anti-CD127 agent is defined according to any one of paragraphs 6 to 10.

What is claimed is:

1. A method for treating a patient having a CD127 positive cancer, the method comprising administering to the patient an effective amount of an anti-CD127 agent,
wherein the anti-CD127 agent (i) comprises Antibody Dependent Cellular Phagocytosis (ADCP) activity against CD127-positive tumor cells but (ii) does not have Antibody Dependent Cytotoxic Activity (ADCC), and
wherein the CD127-positive cancer cancer is treated by the phagocytosis of CD127-positive tumor cells induced by the anti-CD127 agent.

2. The method of claim 1, wherein the CD127-positive cancer is a CD127-positive leukemia or a cancer with solid CD127-positive tumors.

3. The method of claim 1, wherein the anti-CD127 agent induces the phagocytosis of CD127-positive tumor cells by macrophages.

4. The method of claim 1, wherein the anti-CD127 agent has ADCP activity against CD127-positive tumor cells by macrophages.

5. The method of claim 1, wherein the the anti-CD127 agent does not have ADCC activity against immune cells.

6. The method of claim 5, wherein the the anti-CD127 agent does not have ADCC activity against T cells.

7. The method of claim 1, wherein the anti-CD127 agent is an anti-CD127 antibody or antigen-binding fragment thereof or an antigen-binding antibody mimetic.

8. The method of claim 1, wherein the CD127-positive cancer is Leukemia, Acute Lymphoblastic Leukemia (ALL), T-cell ALL or B-cell ALL.

9. The method of claim 1, wherein the CD127-positive cancer is a solid cancer, in particular a mesothelioma.

10. The method according to claim 1, wherein the anti-CD127 agent does not induce lymphodepletion in said patient.

11. The method according to claim 1, wherein the anti-CD127 agent is an anti-CD127 antibody or antigen-binding fragment thereof, comprising a constant chain belonging to the subclass of IgG1, IgG2, IgG3 or IgG4, in particular the subclass of mammalian IgG1, IgG2, IgG3 or IgG4, more particularly the subclass of mammalian IgG4.

12. The method according to claim 1, wherein the anti-CD127 agent is selected from the group consisting of chimeric antibodies, humanized antibodies and fully-human monoclonal antibodies.

13. The method according to claim 1, wherein the anti-CD127 agent is an anti-CD127 antibody or antigen-binding fragment thereofcomprising:
a. a VH chain comprising:
a VHCDR1 comprising the amino acid sequence of SEQ ID No. 3;
a VHCDR2 comprising the amino acid sequence of SEQ ID No. 4; and
a VHCDR3 comprising the amino acid sequence of SEQ ID No. 5 or SEQ ID No. 6; and
b. a VL chain comprising at least the following amino acid sequences:
a VLCDR1 comprising the amino acid sequence of SEQ ID No. 7 or SEQ ID No. 8;
a VLCDR2 comprising the amino acid sequence of SEQ ID No. 9 or SEQ ID No. 10; and
a VLCDR3 comprising the amino acid sequence of SEQ ID No. 11.

14. The method according to claim 1, wherein the anti-CD127 agent is an antagonist of the IL-7R signaling pathway induced by the binding of IL-7 to CD127.

15. The method according to claim 1, wherein the anti-CD127 agent is used in combination with a conventional treatment of cancer.

16. The method according to claim 1, wherein administering the anti-CD127 agent is performed in combination with at least one second therapeutic agent selected from the group consisting of a chemotherapeutic agent, a targeted cancer therapy agent, an immunotherapeutic agent and a radiotherapy agent.

17. The method according to claim 16, wherein administering the anti-CD127 agent and the at least one second therapeutic agent is performed simultaneously, separately or sequentially.

18. The method according to claim 16, wherein the at least one second therapeutic agent is selected from the group consisting of a cytotoxic agent, a chemotherapeutic agent, an anti-angiogenic agent, a cell-cycle control/apoptosis regulating agent, a hormonal regulating agent and an anti-cancer immunogen agent, in particular an anti-cancer antibody, more particularly a tumor-targeting antibody.

19. The method according to claim 16, wherein the at least one second therapeutic agent is selected from the group consisting of an anti-CD3 agent, in particular anti-CD3 antibody, an anti-PD1 agent, in particular an anti-PD1 antibody, an anti-PDL1 agent, in particular an anti-PDL1 antibody, an anti-CTLA4 agent, in particular an anti-CTLA4 antibody, an agonist of CD137, in particular an agonist anti-CD137 antibody, an anti-VEGF agent, in particular an anti-VEGF antibody, an anti-CLEC-1 agent, in particular an anti-CLEC-1 antibody, an anti-CD28 agent, in particular an anti-CD28 antibody, an anti-CD19 agent, in particular an anti-CD19 antibody, and anti-CD47 agent, in particular an anti-CD47 antibody, an anti-SIRPa agent, in particular an anti-SIRPa antibody, an anti-Bcl-2 agent, in particular venetoclax, an inhibitor of the tyrosine/kinase pathway, Dexamethasone, rituximab, trastuzumab, cetuximab, Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Vincristine, Marqibo (Vincristine Sulfate Liposome); Mercaptopurine (or Purinethol or Purixan); Methotrexate Sodium (or Trexall); Nelarabine; Oncaspar (or Pegasparagase or PEG-Asparaginase); Ponatinib Hydrochloride; Prednisone; Purinethol (Mercaptopurine); Vincristine Sulfate, Vincristine Sulfate Liposome.

20. The method according to claim 16, wherein the at least one second therapeutic agent is Dexamethasone and/or Oncaspar (or Pegaspargase or PEG-Asparaginase) and/or Vincristine, or Dexamethasone and Oncaspar (or Pegaspargase or PEG-Asparaginase) and Vincristine.

21. The method according to claim 1, wherein the anti-CD127 agent is administered to said patient after the patient has been evaluated as having CD127-positive tumor cells.

22. A method for promoting phagocytosis of CD127-positive tumor cells, the method comprising administering to a patient in need thereof an effective amount of an anti-CD127 agent, wherein the anti-CD127 agent(i) comprises Antibody Dependent Cellular Phagocytosis (ADCP) activity against CD127-positive tumor cells but (ii) does not have Antibody Dependent Cytotoxic Activity (ADCC).

23. The method of claim 22, wherein the patient has a CD127-positive cancer which is a CD127-positive leukemia or a cancer with solid CD127-positive tumors.

24. The method of claim 22, wherein the anti-CD127 agent induces the phagocytosis of CD127-positive tumor cells by macrophages.

25. The method of claim 22, wherein the anti-CD127 agent has ADCP activity against CD127-positive tumor cells by macrophages.

26. The method of claim 22, wherein the the anti-CD127 agent does not have ADCC activity against immune cells.

27. The method of claim 22, wherein the the anti-CD127 agent does not have ADCC activity against T cells.

28. The method of claim 22, wherein the anti-CD127 agent is a an anti-CD127 antibody or antigen-binding fragment thereof or antigen-binding antibody mimetic.

29. The method of claim 22, wherein the CD127-positive cancer is Leukemia, Acute Lymphoblastic Leukemia (ALL), T-cell ALL or B-cell ALL.

30. The method of claim 29, wherein the CD127-positive cancer is selected from the group consisting of CD127 overexpressing Acute Lymphoblastic Leukemia (ALL), CD127 and/or JAK-STAT pathway mutated ALL, BCR-ABL1—like ALL, and B cell precursor ALL bearing one the following cytogenetics: t(1;19), t(12,21), MLL-rearrangements, hyperdiploid karyotypes, trisomy 4, and trisomy10.

31. The method of claim 22, wherein the CD127-positive cancer is a solid cancer, in particular mesothelioma.

32. The method according to claim 22, wherein the anti-CD127 agent does not induce lymphodepletion in said patient.

33. The method according to claim 22, wherein the anti-CD127 agent is an anti-CD127 antibody or antigen-binding fragment thereof, comprising a constant chain belonging to the subclass of IgG1, IgG2, IgG3 or IgG4, in particular the subclass of mammalian IgG1, IgG2, IgG3 or IgG4, more particularly the subclass of mammalian IgG4.

34. The method according to claim 22, wherein the anti-CD127 agent is selected from the group consisting of chimeric antibodies, humanized antibodies and fully-human monoclonal antibodies.

35. The method according to claim 22, wherein the anti-CD127 agent is an anti-CD127 antibody or antigen-binding fragment thereofcomprising:
a. a VH chain comprising:
a VHCDR1 comprising the amino acid sequence of SEQ ID No. 3;
a VHCDR2 comprising the amino acid sequence of SEQ ID No. 4; and
a VHCDR3 comprising the amino acid sequence of SEQ ID No. 5 or SEQ ID No. 6; and
b. a VL chain comprising at least the following amino acid sequences:
a VLCDR1 comprising the amino acid sequence of SEQ ID No. 7 or SEQ ID No. 8;
a VLCDR2 comprising the amino acid sequence of SEQ ID No. 9 or SEQ ID No. 10; and
a VLCDR3 comprising the amino acid sequence of SEQ ID No. 11.

36. The method according to claim 22, wherein the anti-CD127 agent is an antagonist of the IL-7R signaling pathway induced by the binding of IL-7 to CD127.

37. The method according to claim 22, wherein the anti-CD127 agent is used in combination with a conventional treatment of cancer.

38. The method according to claim 22, wherein administering the anti-CD127 agent is performed in combination with at least one second therapeutic agent selected from the group consisting of a chemotherapeutic agent, a targeted cancer therapy agent, an immunotherapeutic agent and a radiotherapy agent.

39. The method according to claim 38, wherein administering the anti-CD127 agent and the at least one second therapeutic agent is performed simultaneously, separately or sequentially.

40. The method according to claim 38, wherein the at least one second therapeutic agent is selected from the group consisting of a cytotoxic agent, a chemotherapeutic agent, an anti-angiogenic agent, a cell-cycle control/apoptosis regulating agent, a hormonal regulating agent and an anti-cancer immunogen agent, in particular an anti-cancer antibody, more particularly a tumor-targeting antibody.

41. The method according to claim 38, wherein the at least one second therapeutic agent is selected from the group consisting of an anti-CD3 agent, in particular anti-CD3 antibody, an anti-PD1 agent, in particular an anti-PD1 antibody, an anti-PDL1 agent, in particular an anti-PDL1 antibody, an anti-CTLA4 agent, in particular an anti-CTLA4 antibody, an agonist of CD137, in particular an agonist anti-CD137 antibody, an anti-VEGF agent, in particular an anti-VEGF antibody, an anti-CLEC-1 agent, in particular an anti-CLEC-1 antibody, an anti-CD28 agent, in particular an anti-CD28 antibody, an anti-CD19 agent, in particular an anti-CD19 antibody, and anti-CD47 agent, in particular an anti-CD47 antibody, an anti-SIRPa agent, in particular an anti-SIRPa antibody, an anti-Bcl-2 agent, in particular venetoclax, an inhibitor of the tyrosine/kinase pathway, Dexamethasone, rituximab, trastuzumab, cetuximab, Arranon (Nelarabine); Asparaginase Erwinia chrysanthemi (or Erwinaze), Asparlas (or Calaspargase Pegol-mknl); Besponsa (Inotuzumab Ozogamicin); Blinatumomab (or Blincyto); and Cerubidine (or Daunorubicin Hydrochloride or Rubidomycin); Clofarabine (or Clolar); Cyclophosphamide; Cytarabine; Dasatinib (or Sprycel); Doxorubicin Hydrochloride; Gleevec (Imatinib Mesylate); Iclusig (Ponatinib Hydrochloride); Inotuzumab Ozogamicin; Imatinib Mesylate; Kymriah (or Tisagenlecleucel); Vincristine, Marqibo (Vincristine Sulfate Liposome); Mercaptopurine (or Purinethol or Purixan); Methotrexate Sodium (or Trexall); Nelarabine; Oncaspar (or Pegasparagase or PEG-Asparaginase); Ponatinib Hydrochloride; Prednisone; Purinethol (Mercaptopurine); Vincristine Sulfate, Vincristine Sulfate Liposome.

42. The method according to claim 38, wherein the at least one second therapeutic agent is Dexamethasone and/or Oncaspar (or Pegaspargase or PEG-Asparaginase) and/or Vincristine, or Dexamethasone and Oncaspar (or Pegaspargase or PEG-Asparaginase) and Vincristine.

43. The method according to claim 22, wherein the anti-CD127 agent is administered to said patient after the patient has been evaluated as having CD127-positive tumor cells.


SEQUENCE LISTING (32 sequences)

SEQ ID NO: 1 - protein, 459 aa, synthetic construct
  REGION 1..459: note = synthetic sequence
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 2 - protein, 219 aa, synthetic construct
  REGION 1..219: note = synthetic sequence
ESGYAQNGDLEDAELDDYSFSCYSQLEVNGSQHSLTCAFEDPDVNTTNLEFEICGALVEVKCLNFRKLQEIYFIETKKFLLIGKSNICVKVGEKSLTCKKIDLTTIVKPEAPFDLSVIYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 3 - protein, 9 aa, synthetic construct
  REGION 1..9: note = synthetic sequence
FTLSDYYMA

SEQ ID NO: 4 - protein, 17 aa, synthetic construct
  REGION 1..17: note = synthetic sequence
TISASGLRTYYPDSVKG

SEQ ID NO: 5 - protein, 13 aa, synthetic construct
  REGION 1..13: note = synthetic sequence
PMSAHYGFNYFDY

SEQ ID NO: 6 - protein, 13 aa, synthetic construct
  REGION 1..13: note = synthetic sequence
PLSAHYGFNYFDY

SEQ ID NO: 7 - protein, 11 aa, synthetic construct
  REGION 1..11: note = synthetic sequence
RTSEDIYNGLA

SEQ ID NO: 8 - protein, 12 aa, synthetic construct
  REGION 1..12: note = synthetic sequence
CRTSEDIYQGLA

SEQ ID NO: 9 - protein, 7 aa, synthetic construct
  REGION 1..7: note = synthetic sequence
SANSLHI

SEQ ID NO: 10 - protein, 7 aa, synthetic construct
  REGION 1..7: note = synthetic sequence
SANTLHI

SEQ ID NO: 11 - protein, 9 aa, synthetic construct
  REGION 1..9: note = synthetic sequence
QQYYDYPLA

SEQ ID NO: 12 - protein, 122 aa, synthetic construct
  REGION 1..122: note = synthetic sequence
AVQLVESGGGLVQPGGSLKLSCAVSGFTLSDYYMAWVRQAPKKGLEWVATISASGLRTYYPDSVKGRFTISRDDAKRSLFLQMTSLKSEDTATYYCARPMSAHYGFNYFDYWGQGVMVTVSS

SEQ ID NO: 13 - protein, 122 aa, synthetic construct
  REGION 1..122: note = synthetic sequence
QVQLVESGGGLVKPGGSLRLSCAVSGFTLSDYYMAWVRQAPGKGLEWVATISASGLRTYYPDSVKGRFTISRDDAKNSLYLQMNSLRAEDTAVYYCARPMSAHYGFNYFDYWGQGTLVTVSS

SEQ ID NO: 14 - protein, 122 aa, synthetic construct
  REGION 1..122: note = synthetic sequence
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPMSAHYGFNYFDYWGQGTLVTVSS

SEQ ID NO: 15 - protein, 122 aa, synthetic construct
  REGION 1..122: note = synthetic sequence
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPLSAHYGFNYFDYWGQGTLVTVSS

SEQ ID NO: 16 - protein, 108 aa, synthetic construct
  REGION 1..108: note = synthetic sequence
DIQMTQSPASLSASLGETVTIECRTSEDIYNGLAWYQQKPGKSPQLLVYSANSLHIGVPSRFSGSGSGTQYSLKINSLQFEDVASYFCQQYYDYPLAFGSGTKLEIKR

SEQ ID NO: 17 - protein, 107 aa, synthetic construct
  REGION 1..107: note = synthetic sequence
EIVMTQSPATLSVSPGERATLSCRTSEDIYNGLAWYQQKPGQAPRLLVYSANSLHIGIPARFSGSGSGTEYTLTISSLQSEDFAVYFCQQYYDYPLAFGGGTKVEIK

SEQ ID NO: 18 - protein, 107 aa, synthetic construct
  REGION 1..107: note = synthetic sequence
EIVMTQSPATLSVSPGERATLSCRTSEDIYNGLAWYQQKPGQAPRLLIYSANSLHIGIPARFSGSGSGTEFTLTISSLQSEDFAVYYCQQYYDYPLAFGGGTKVEIK

SEQ ID NO: 19 - protein, 107 aa, synthetic construct
  REGION 1..107: note = synthetic sequence
EIVMTQSPATLSVSPGERATLSCRTSEDIYQGLAWYQQKPGQAPRLLIYSANTLHIGIPARFSGSGSGTEFTLTISSLQSEDFAVYYCQQYYDYPLAFGGGTKVEIK

SEQ ID NO: 20 - protein, 449 aa, synthetic construct
  REGION 1..449: note = synthetic sequence
QVQLVESGGGLVKPGGSLRLSCAVSGFTLSDYYMAWIRQAPGKGLEWVSTISASGLRTYYPDSVKGRFTISRDNAKNSLYLQMNSLRAEDTAVYYCARPLSAHYGFNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 21 - protein, 213 aa, synthetic construct
  REGION 1..213: note = synthetic sequence
EIVMTQSPATLSVSPGERATLSCRTSEDIYQGLAWYQQKPGQAPRLLIYSANTLHIGIPARFSGSGSGTEFTLTISSLQSEDFAVYYCQQYYDYPLAFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 122 aa, synthetic construct; identical to SEQ ID NO: 15
  REGION 1..122: note = synthetic sequence

SEQ ID NO: 23 - protein, 107 aa, synthetic construct
  REGION 1..107: note = synthetic sequence
DIQMTQSPSSLSASVGDRVTITCRTSEDIYQGLAWYQQKPGKAPKLLVYSANTLHIGVPSRFSGSGSGTDYTLTISSLQPEDFATYFCQQYYDYPLAFGGGTKVEIK

SEQ ID NO: 24 - protein, 107 aa, synthetic construct
  REGION 1..107: note = synthetic sequence
DIQMTQSPSSLSASVGDRVTITCRTSEDIYQGLAWYQQKPGKAPKLLLYSANTLHIGVPSRFSGSGSGTDYTLTISSLQPEDFATYFCQQYYDYPLAFGGGTKVEIK

SEQ ID NO: 25 - protein, 107 aa, synthetic construct
  REGION 1..107: note = synthetic sequence
DIQMTQSPSSLSASVGDRVTITCRTSEDIYQGLAWYQQKPGKAPKLLVYSANTLHIGVPSRFSGSGSGTDYTLTISSLQPEDFATYYCQQYYDYPLAFGGGTKVEIK

SEQ ID NO: 26 - protein, 107 aa, synthetic construct
  REGION 1..107: note = synthetic sequence
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIK

SEQ ID NO: 27 - protein, 449 aa, synthetic construct; identical to SEQ ID NO: 20
  REGION 1..449: note = synthetic sequence

SEQ ID NO: 28 - protein, 214 aa, synthetic construct
  REGION 1..214: note = synthetic sequence
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 213 aa, synthetic construct
  REGION 1..213: note = synthetic sequence
DIQMTQSPSSLSASVGDRVTITCRTSEDIYQGLAWYQQKPGKAPKLLLYSANTLHIGVPSRFSGSGSGTDYTLTISSLQPEDFATYYCQQYYDYPLAFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 327 aa, synthetic construct
  REGION 1..327: note = synthetic sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 107 aa, synthetic construct [sequence omitted]

SEQ ID NO: 32 - protein, 106 aa, synthetic construct
  REGION 1..106: note = synthetic sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC